(19) United States
(12) Patent Application Publication
(10) Pub. No.: US 2003/0027286 A1
(43) Pub. Date: Feb. 6, 2003

(54) Bacterial promoters and methods of use

(76) Inventors: Robert Haselbeck, San Diego, CA (US); Daniel Wall, San Diego, CA (US); Molly Gross, San Diego, CA (US)

Correspondence Address: KNOBBE MARTENS OLSON & BEAR LLP, 620 NEWPORT CENTER DRIVE, SIXTEENTH FLOOR, NEWPORT BEACH, CA, 92660, US

(21) Appl. No.: 10/032,393

(22) Filed: Dec. 21, 2001

Related U.S. Application Data
(60) Provisional application No. 60/259,434, filed on Dec. 27, 2000.
(60) Provisional application No. 60/230,335, filed on Sep. 6, 2000.

(51) Int. Cl. C07H 21/04; C12P 21/04; C12N 9/50; C12Q 1/68; C12N 1/21; C12N 15/74

(52) U.S. Cl. 435/69.6; 435/219; 435/320.1; 435/252.3; 536/23.2; 435/6

(57) ABSTRACT

Compositions and methods are disclosed herein that relate to the development of fusion promoters for regulating gene expression in bacteria. Embodiments include fusion promoters comprising one or more operators linked to a promoter that is modified to have altered activity in Gram-positive organisms. Vectors and cells containing these fusion promoters are also described. Other embodiments include, methods of using these fusion promoters to regulate nucleic acid and/or polypeptide expression, methods of using these fusion promoters to identify proliferation-required genes, and methods of using these fusion promoters to identify molecules having potential antibiotic activity.

### RELATED APPLICATIONS

This application claims priority to U.S. Provisional Patent Application No. 60/259434, filed Dec. 27, 2000, by Robert Haselbeck, and entitled “BACTERIAL PROMOTERS AND METHODS OF USE” and U.S. patent application Ser. No. 09/948,993, filed Sep. 6, 2001, by Daniel Tishkoff, and entitled “RAPID METHOD FOR REGULTING GENE EXPRESSION”, which claims priority to U.S. Provisional Patent Application Serial No. 60/230,335, filed Sep. 6, 2000, by Daniel Tishkoff, and entitled “RAPID REPLACEMENT OF GENOMIC PROMOTERS TO GENERATE STRAINS FOR USE IN A CELL-BASED ASSAY FOR ANTIBIOTICS”, the disclosures of which are incorporated herein by reference in their entirety.

### FIELD OF THE INVENTION

The invention described herein relates to fusion promoters which comprise one or more promoters and one or more operators for directing transcription in Gram-positive bacteria. More specifically, some embodiments relate to fusion promoters comprising a promoter sequence that has been modified so as to alter its transcription activity in Gram-positive organisms linked to a xylose operator, cells having constructs that include such fusion promoters, and methods of using of these manufactures.

### BACKGROUND OF THE INVENTION

Genetic information is expressed in bacteria by a series of steps involving transcription of DNA into mRNA and the subsequent translation of the mRNA into polypeptides or proteins. The expression of encoded information is initiated at a region on the DNA molecule called a “promoter”, onto which RNA polymerase recognizes and, thereby, initiates transcription. Promoters, which usually precede protein-coding nucleotide sequences (“structural genes”), vary in “strength” depending on the ability to recognize and bind RNA polymerase in a manner facilitating the initiation of transcription.

The regulation of transcription levels from certain promoters in bacteria can be facilitated by “operators” that are positioned adjacent to or within the promoter, Operator sequences generally function by interacting with modulator proteins, which either activate or repress the transcriptional activity of the promoter. Thus, the inherent strength of the promoter and the amount of regulation that can be imposed upon the promoter by an associated operator sequence interacting with the modulator protein contribute significantly to the control of transcription.

Current techniques in molecular biology enable the transfer to bacteria of exogenous nucleic acids, which one desires to transcribe. Successful transcription of the transferred nucleic acid in the host microorganism, however, depends on the association of the transferred nucleic acid with a suitable promoter and/or operator sequence. Frequently, the promoter and/or operator that is naturally associated with the nucleic acid to be transcribed will not support desired levels of transcription in the new host and, a new promoter and/or operator sequence must be operably linked to the nucleic acid. A variety of promoters and operators have been studied in this regard. (See e.g., von Gabain, et al., Proc. Natl. Acad. Sci. (USA), 76:189-193 (1979); Casadaban, et al., J. Molecular Biology, 138:179-207 (1980); West, et al., Gene, 9:175-193 (1980); (Chang et al., Nature, 275:615 (1978); Itakura et al., Science, 198:1056 (1977); Goeddel et al., Nature, 281:544 (1979); and (Goeddel et al., Nucleic Acids Res., 8:4057 (1980); and EPO Application Publication No. 0036776). However, many of the currently available regulatable promoters are designed for use in Gram-negative bacteria, such as E. coli. There remains a need for regulatable promoters that function in Gram-positive bacteria. In particular, the need exists for promoters that can be used to achieve the precise levels of nucleic acid and/or protein expression desired for applications such as cell-based gene and drug discovery in Gram-positive bacteria. For example, in the discovery of proliferation-required genes, the expression of a proliferation-required mRNA which is transcibed at a low level may be sufficiently reduced by the levels of antisense transcript produced from a promoter having a low maximal level of transcriptional activity to inhibit cell growth. However, the expression of a proliferation-required mRNA which is transcribed at higher levels may only be sufficiently reduced by levels of antisense transcript that can produced from a promoter having a high maximal transcriptional activity. As a result, the number of types of proliferation-required genes that can be discovered are determined in part by the transcriptional activity of the promoter used to express the proliferation-inhibiting antisense RNA. Accordingly, there exists a need for a promoter suite having a wide dynamic range of transcriptional activities in Gram-positive bacteria. In methods in which genes required for proliferation of Gram-positive organisms are identified by transcribing an antisense nucleic acid which is complementary to at least a portion of such genes, the endogenous transcription levels of the genes may vary. Accordingly, it is valuable to have a suite of promoters which provide a wide range of antisense nucleic acid transcription levels to ensure that a sufficient amount of antisense nucleic acid is obtained to allow the identification of proliferation-required genes which are normally expressed at high levels in the Gram-positive organism. In drug discovery applications which utilize Gram-positive cells that are sensitized by the expression of proliferation-inhibiting antisense RNAs, the degree to which the cells are sensitized is an important factor in determining the candidate compounds that are discovered. Furthermore, the expression of some proliferation-required target genes may be so greatly reduced by the production of proliferation-inhibiting antisense RNA from a highly active promoter that, rather than becoming sensitized, the cell dies. Accordingly, there exists a need for a promoter suite containing a collection of promoters which provide a wide range of basal transcription levels, maximal transcription levels, and degrees of induction which can be used to obtain a level of antisense nucleic acid which is optimal for the identification of a particular gene as being required for proliferation or optimal for identifying compounds which act on the gene product of a particular gene which is required for proliferation in cell-based assays using sensitized cells as described herein. Thus, there remains a need for a suite of promoters having the ability to tightly and/or finely regulate transcription in Gram-positive bacteria.

### BRIEF SUMMARY OF THE INVENTION

Some aspects of the present invention are described in the numbered paragraphs below.

1. An isolated nucleic acid comprising a fusion promoter said fusion promoter comprising at least one promoter that is modified to have altered activity in at least one gram-positive organism said promoter being linked to at least one operator selected from the group consisting of xylO, tetO, trpO, malO and λc1O, wherein said at least one operator is positioned such that binding of at least one repressor to said at least one operator represses transcription from said fusion promoter.

2. The isolated fusion promoter of claim 1, wherein said at least one promoter is selected from the group consisting of SEQ ID NOs.: 36-45.

3. The isolated nucleic acid of claim 1 wherein said at least one operator is xylO.

4. The isolated nucleic acid of claim 3, wherein said at least one promoter is T5.

5. The isolated nucleic acid of claim 3, further comprising a second operator.

6. The isolated nucleic acid of claim 5, wherein said second operator is lacO.

7. The isolated nucleic acid of claim 1, wherein said fusion promoter is responsive to an inducer.

8. The isolated nucleic acid of claim 7, wherein said inducer is xylose.

9. The isolated nucleic acid of claim 1, wherein said fusion promoter is titratable.

10. The isolated nucleic acid of claim 1, wherein said at least one gram-positive organism is selected from the group consisting of Bacillus anthracis, Clostridium botulinum, Clostridium difficile, Clostridium perfringens, Clostridium teteni, Corynebacterium diptheriae, Enterococcus faecalis, Enterococcus faecium, Lactococcus lactis, Listeria monocytogenes, Mycobacterium leprae, Mycobacterium tuberculosis, Nocardia asteroides, Staphylococcus aureus, Staphylococcus epidermidis, Staphylococcus xylosis, Streptococcus pneumoniae, Streptococcus mutans and any species falling within the genera of any of the above species.

11. The isolated nucleic acid of claim 1, wherein said at least one gram-positive organism is Staphylococcus aureus.

12. The isolated nucleic acid of claim 1, wherein said at least one gram-positive organism is Enterococcus faecalis.

13. An isolated nucleic acid comprising a fusion promoter said fusion promoter comprising at least one promoter selected from the group consisting of T5, CP25, P32, P59, P1P2 and PL, said promoter being linked to at least one operator selected from the group consisting of xylO, tetO, trpO, malO and λc1O, wherein said at least one operator is positioned such that binding of at least one repressor to said at least one operator represses transcription from said fusion promoter.

14. The isolated nucleic acid of claim 13, wherein said at least one operator is xylO.

15. The isolated nucleic acid of claim 14, wherein said at least one promoter is T5.

16. The isolated nucleic acid of claim 14, further comprising a second operator.

17. The isolated nucleic acid of claim 16, wherein said second operator is lacO.

18. The isolated nucleic acid of claim 13, wherein said fusion promoter is responsive to an inducer.

19. The isolated nucleic acid of claim 18, wherein said inducer is xylose.

20. The isolated nucleic acid of claim 13, wherein said fusion promoter is titratable.

21. The isolated nucleic acid of claim 13, wherein said at least one gram-positive organism is selected from the group consisting of Bacillus anthracis, Clostridium botulinum, Clostridium difficile, Clostridium perfringens, Clostridium teteni, Corynebacterium diptheriae, Enterococcus faecalis, Enterococcus faecium, Lactococcus lactis, Listeria monocytogenes, Mycobacterium leprae, Mycobacterium tuberculosis, Nocardia asteroides, Staphylococcus aureus, Staphylococcus epidermidis, Staphylococcus xylosis, Streptococcus pneumoniae, Streptococcus mutans and any species falling within the genera of any of the above species.

22. The isolated nucleic acid of claim 13, wherein said at least one gram-positive organism is Staphylococcus aureus.

23. The isolated nucleic acid of claim 13, wherein said at least one gram-positive organism is Enterococcus faecalis.

24. An isolated fusion promoter comprising one of SEQ ID NO.: 26-35.

25. A vector comprising the isolated nucleic acid of claim 1.

26. A vector comprising the isolated nucleic acid of claim 13.

27. A vector comprising the isolated nucleic acid of claim 24.

28. The vector of claim 25 further comprising at least one replicon selected from the group consisting of p15a, pC194 and pCT1138.

29. The vector of claim 25 further comprising a reporter gene operably linked to said fusion promoter.

30. The vector of claim 29, wherein said reporter gene is lacL-lacM.

31. The vector of claim 30, wherein lacL-lacM is derived from Leuconostoc mesenteroides.

32. The vector of claim 25, wherein said at least one operator is xylO.

33. The vector of claim 32, wherein said at least one promoter is T5.

34. The vector of claim 32, further comprising a second operator.

35. The vector of claim 34, wherein said second operator is lacO.

36. The vector of claim 25, wherein said fusion promoter is responsive to an inducer.

37. The vector of claim 36, wherein said inducer is xylose.

38. The vector of claim 25, wherein said fusion promoter is titratable.

39. The vector of claim 25, wherein said at least one gram-positive organism is selected from the group consisting of Bacillus anthracis, Clostridium botulinum, Clostridium difficile, Clostridium perfringens, Clostridium teteni, Corynebacterium diptheriae, Enterococcus faecalis, Enterococcus faecium, Lactococcus lactis, Listeria monocytogenes, Mycobacterium leprae, Mycobacterium tuberculosis, Nocardia asteroides, Staphylococcus aureus, Staphylococcus epidermidis, Staphylococcus xylosis, Streptococcus pneumoniae, Streptococcus mutans and any species falling within the genera of any of the above species.

40. The vector of claim 25, wherein said at least one gram-positive organism is Staphylococcus aureus.

41. The vector of claim 25, wherein said at least one gram-positive organism is Enterococcus faecalis.

42. The vector of claim 25, further comprising a random fragment of a microbial genome operably linked to said fusion promoter.

43. The vector of claim 25, further comprising a nucleic acid that encodes a peptide, wherein said nucleic acid is operably linked to said fusion promoter.

44. The vector of claim 25, further comprising a nucleic acid that is complementary to a portion of a microbial genome, wherein said nucleic acid is operably linked to said fusion promoter.

45. The vector of claim 25, further comprising a nucleic acid that encodes a molecule that inhibits the proliferation of microbe, wherein said nucleic acid is operably linked to said fusion promoter.

46. A host cell comprising the nucleic acid of claim 1.

47. A host cell comprising the nucleic acid of claim 13.

48. A method of expressing a biomolecule in a cell said method comprising:

(a) introducing a construct into a cell said construct comprising a fusion promoter operably linked to a nucleic acid encoding a biomolecule, said fusion promoter comprising at least one promoter that is modified to have altered activity in at least one gram-positive organism said promoter being linked to at least one operator selected from the group consisting of xylO, tetO, trpO, malO and λc1O, wherein said at least one operator is positioned such that binding of at least one repressor to said at least one operator represses transcription from said fusion promoter; and

(b) transcribing said nucleic acid encoding a biomolecule from said fusion promoter.

49. The method of claim 48, wherein said at least one operator is xylO.

50. The method of claim 49, wherein said at least one promoter is T5.

51. The method of claim 49, wherein said fusion promoter further comprises a second operator.

52. The method of claim 51, wherein said second operator is lacO.

53. The method of claim 48, wherein said fusion promoter is responsive to an inducer.

54. The method of claim 53, wherein said inducer is xylose.

55. The method of claim 48, wherein said fusion promoter is titratable.

56. The method of claim 48, wherein said at least one gram-positive organism is selected from the group consisting of Bacillus anthracis, Clostridium botulinum, Clostridium difficile, Clostridium perfringens, Clostridium teteni, Corynebacterium diptheriae, Enterococcus faecalis, Enterococcus faecium, Lactococcus lactis, Listeria monocytogenes, Mycobacterium leprae, Mycobacterium tuberculosis, Nocardia asteroides, Staphylococcus aureus, Staphylococcus epidermidis, Staphylococcus xylosis, Streptococcus pneumoniae, Streptococcus mutans and any species falling within the genera of any of the above species.

57. The method of claim 48, wherein said at least one gram-positive organism is Staphylococcus aureus.

58. The method of claim 48, wherein said at least one gram-positive organism is Enterococcus faecalis.

59. The method of claim 48, wherein said nucleic acid is a random fragment of a microbial genome.

60. The method of claim 48, wherein said biomolecule is a peptide or nucleic acid.

61. The method of claim 48, wherein said biomolecule is a nucleic acid that is complementary to a portion of a microbial genome.

62. The method of claim 48, wherein said biomolecule is a nucleic acid that encodes a molecule that inhibits the proliferation of a microbe.

63. A biomolecule produced by the method of claim 48.

64. A method for identifying genes involved in cellular proliferation said method comprising the steps of:

(a) introducing into cells of a cell population a construct comprising an inducible fusion promoter operably linked to a nucleic acid, said fusion promoter comprising at least one promoter that is modified to have altered activity in at least one gram-positive organism said promoter being linked to at least one operator selected from the group consisting of xylO, tetO, trpO, malO and λc1O, wherein said at least one operator is positioned such that binding of at least one repressor to said at least one operator represses transcription from said fusion promoter;

(b) inducing transcription of said nucleic acid from said inducible fusion promoter;

(c) identifying the cells in said cell population whose proliferation is reduced in response to the induction of transcription of said nucleic acid; and

(d) identifying the gene from a cell identified in step (c) to which at least a portion of said nucleic acid is complementary.

65. A method for identifying genes involved in cellular proliferation said method comprising the steps of:

(a) introducing into cells of a cell population a construct comprising an inducible fusion promoter operably linked to a nucleic acid, said fusion promoter comprising at least one promoter selected from the group consisting of T5, CP25, P32, P59, P1P2 and PL, said promoter being linked to at least one operator selected from the group consisting of xylO, tetO, trpO, malO and λc1O, wherein said at least one operator is positioned such that binding of at least one repressor to said at least one operator represses transcription from said fusion promoter;

(b) inducing transcription of said nucleic acid from said inducible fusion promoter;

(c) identifying the cells in said cell population whose proliferation is reduced in response to the induction of transcription of said nucleic acid; and

(d) identifying the gene from a cell identified in step (c) to which at least a portion of said nucleic acid is complementary.

66. The method of claim 64, wherein said nucleic acid is random fragment of a microbial genome.

67. The method of claim 64, wherein said nucleic acid encodes an aptamer.

68. The method of claim 64, wherein said nucleic acid encodes a transcript that is complementary to a portion of a microbial genome.

69. The method of claim 64, wherein said nucleic acid encodes a molecule that inhibits the proliferation of a microbe.

70. The method of claim 64, wherein said at least one operator is xylO.

71. The method of claim 70, wherein said fusion promoter is T5.

72. The method of claim 70, wherein said fusion promoter further comprises a second operator.

73. The method of claim 72, wherein said second operator is lacO.

74. The method of claim 64, wherein said inducer is xylose.

75. The method of claim 64, wherein said fusion promoter is titratable.

76. The method of claim 64, wherein said at least one gram-positive organism is selected from the group consisting of Bacillus anthracis, Clostridium botulinum, Clostridium difficile, Clostridium perfringens, Clostridium teteni, Corynebacterium diptheriae, Enterococcus faecalis, Enterococcus faecium, Lactococcus lactis, Listeria monocytogenes, Mycobacterium leprae, Mycobacterium tuberculosis, Nocardia asteroides, Staphylococcus aureus, Staphylococcus epidermidis, Staphylococcus xylosis, Streptococcus pneumoniae, Streptococcus mutans and any species falling within the genera of any of the above species.

77. The method of claim 64, wherein said at least one gram-positive organism is Staphylococcus aureus.

78. The method of claim 64, wherein said at least one gram-positive organism is Enterococcus faecalis.

79. A method for identifying genes involved in cellular proliferation said method comprising the steps of:

(a) introducing into the genome of a cell a construct comprising an inducible fusion promoter operably linked to a nucleic acid, said fusion promoter comprising at least one promoter that is modified to have altered activity in at least one gram-positive organism said promoter being linked to at least one operator selected from the group consisting of xylO, tetO, trpO, malO and λc1O, wherein said at least one operator is positioned such that binding of at least one repressor to said at least one operator represses transcription from said fusion promoter;

(b) comparing the proliferation of said cell cultured in the presence of a first concentration of an inducer that induces transcription from said fusion promoter with the proliferation of said cell cultured in the presence of a concentration of said inducer that is less than said first concentration, wherein a difference in proliferation indicates that said fusion promoter modulates a gene that is required for proliferation; and

(c) identifying the gene that is modulated by said fusion promoter.

80. A method for identifying genes involved in cellular proliferation said method comprising the steps of:

(a) introducing into the genome of a cell a construct comprising an inducible fusion promoter operably linked to a nucleic acid, said fusion promoter comprising at least one promoter selected from the group consisting of T5, CP25, P32, P59, P1P2 and PL, said promoter being linked to at least one operator selected from the group consisting of xylO, tetO, trpO, malO and λc1O, wherein said at least one operator is positioned such that binding of at least one repressor to said at least one operator represses transcription from said fusion promoter;

(b) comparing the proliferation of said cell cultured in the presence of a first concentration of an inducer that induces transcription from said fusion promoter with the proliferation of said cell cultured in the presence of a concentration of said inducer that is less than said first concentration, wherein a difference in proliferation indicates that said fusion promoter modulates a gene that is required for proliferation; and

(c) identifying the gene that is modulated by said fusion promoter.

81. The method of claim 79, wherein said nucleic acid is random fragment of a microbial genome.

82. The method of claim 79, wherein said nucleic acid encodes an aptamer.

83. The method of claim 79, wherein said nucleic acid encodes a transcript that is complementary to a portion of a microbial genome.

84. The method of claim 79, wherein said nucleic acid encodes a molecule that inhibits the proliferation of a microbe.

85. The method of claim 79, wherein said at least one operator is xylO.

86. The method of claim 85, wherein said at least one promoter is T5.

87. The method of claim 85, wherein said fusion promoter further comprises a second operator.

88. The method of claim 87 wherein said second operator is lacO.

89. The method of claim 79, wherein said inducer is xylose.

90. The method of claim 79, wherein said fusion promoter is titratable.

91. The method of claim 79, wherein said at least one gram-positive organism is selected from the group consisting of Bacillus anthracis, Clostridium botulinum, Clostridium difficile, Clostridium perfringens, Clostridium teteni, Corynebacterium diptheriae, Enterococcus faecalis, Enterococcus faecium, Lactococcus lactis, Listeria monocytogenes, Mycobacterium leprae, Mycobacterium tuberculosis, Nocardia asteroides, Staphylococcus aureus, Staphylococcus epidermidis, Staphylococcus xylosis, Streptococcus pneumoniae, Streptococcus mutans and any species falling within the genera of any of the above species.

92. The method of claim 79, wherein said at least one gram-positive organism is Staphylococcus aureus.

93. The method of claim 79, wherein said at least one gram-positive organism is Enterococcus faecalis.

94. A method for identifying a compound which reduces the activity or level of a gene product required for proliferation of a cell said method comprising:

(a) introducing into a cell a construct comprising an inducible fusion promoter operably linked to a nucleic acid that is complementary to at least a portion of a proliferation-required gene, said fusion promoter comprising at least one promoter that is modified to have altered activity in at least one gram-positive organism said promoter being linked to at least one operator selected from the group consisting of xylO, tetO, trpO, malO and λc1O, wherein said at least one operator is positioned such that binding of at least one repressor to said at least one operator represses transcription from said fusion promoter;

(b) sensitizing said cell by inducing transcription from said fusion promoter;

(c) contacting said sensitized cell with a compound; and

(d) determining the degree to which said compound inhibits proliferation of said sensitized cell relative to a cell which has not been sensitized.

95. A method for identifying a compound which reduces the activity or level of a gene product required for proliferation of a cell said method comprising:

(a) introducing into a cell a construct comprising an inducible fusion promoter operably linked to a nucleic acid that is complementary to at least a portion of a proliferation-required gene, said fusion promoter comprising at least one promoter selected from the group consisting of T5, CP25, P32, P59, P1P2 and PL, said promoter being linked to at least one operator selected from the group consisting of xylO, tetO, trpO, malO and λc1O, wherein said at least one operator is positioned such that binding of at least one repressor to said at least one operator represses transcription from said fusion promoter;

(b) sensitizing said cell by inducing transcription from said fusion promoter;

(c) contacting said sensitized cell with a compound; and

(d) determining the degree to which said compound inhibits proliferation of said sensitized cell relative to a cell which has not been sensitized.

96. The method of claim 94, wherein said at least one operator is xylO.

97. The method of claim 96, wherein said at least one promoter is T5.

98. The method of claim 96, wherein said fusion promoter further comprises a second operator.

99. The method of claim 98, wherein said second operator is lacO.

100. The method of claim 94, wherein said inducer is xylose.

101. The method of claim 94, wherein said fusion promoter is titratable.

102. The method of claim 94, wherein said at least one gram-positive organism is selected from the group consisting of Bacillus anthracis, Clostridium botulinum, Clostridium difficile, Clostridium perfringens, Clostridium teteni, Corynebacterium diptheriae, Enterococcus faecalis, Enterococcus faecium, Lactococcus lactis, Listeria monocytogenes, Mycobacterium leprae, Mycobacterium tuberculosis, Nocardia asteroides, Staphylococcus aureus, Staphylococcus epidermidis, Staphylococcus xylosis, Streptococcus pneumoniae, Streptococcus mutans and any species falling within the genera of any of the above species.

103. The method of claim 94, wherein said at least one gram-positive organism is Staphylococcus aureus.

104. The method of claim 94, wherein said at least one gram-positive organism is Enterococcus faecalis.

105. A compound identified using the method of claim 94.

106. A method for inhibiting the activity or expression of a gene in an operon required for proliferation said method comprising:

(a) introducing into a cell a construct comprising an inducible fusion promoter operably linked to a nucleic acid that is complementary to at least a portion of a proliferation-required operon, said fusion promoter comprising at least one promoter that is modified to have altered activity in at least one gram-positive organism said promoter being linked to at least one operator selected from the group consisting of xylO, tetO, trpO, malO and λc1O, wherein said at least one operator is positioned such that binding of at least one repressor to said at least one operator represses transcription from said fusion promoter; and

(b) inducing transcription from said fusion promoter.

107. A method for inhibiting the activity or expression of a gene in an operon required for proliferation said method comprising:

(a) introducing into a cell a construct comprising an inducible fusion promoter operably linked to a nucleic acid that is complementary to at least a portion of a proliferation-required operon, said fusion promoter comprising at least one promoter selected from the group consisting of T5, CP25, P32, P59, P1P2 and PL, said promoter being linked to at least one operator selected from the group consisting of xylO, tetO, trpO, malO and λc1O, wherein said at least one operator is positioned such that binding of at least one repressor to said at least one operator represses transcription from said fusion promoter; and

(b) inducing transcription from said fusion promoter.

108. The method of claim 106, wherein said at least one operator is xylO.

109. The method of claim 108, wherein said at least one promoter is T5.

110. The method of claim 108, wherein said fusion promoter further comprises a second operator.

111. The method of claim 110, wherein said second operator is lacO.

112. The method of claim 106, wherein said inducer is xylose.

113. The method of claim 106, wherein said fusion promoter is titratable.

114. The method of claim 106, wherein said at least one gram-positive organism is selected from the group consisting of Bacillus anthracis, Clostridium botulinum, Clostridium difficile, Clostridium perfringens, Clostridium teteni, Corynebacterium diptheriae, Enterococcus faecalis, Enterococcus faecium, Lactococcus lactis, Listeria monocytogenes, Mycobacterium leprae, Mycobacterium tuberculosis, Nocardia asteroides, Staphylococcus aureus, Staphylococcus epidermidis, Staphylococcus xylosis, Streptococcus pneumoniae, Streptococcus mutans and any species falling within the genera of any of the above species.

115. The method of claim 106, wherein said at least one gram-positive organism is Staphylococcus aureus.

116. The method of claim 106, wherein said at least one gram-positive organism is Enterococcus faecalis.

117. A method for identifying the biological pathway in which a proliferation-required gene or its gene product lies said method comprising:

(a) introducing into a test cell a construct comprising an inducible fusion promoter operably linked to a nucleic acid that is complementary to at least a portion of a proliferation-required gene, said fusion promoter comprising at least one promoter that is modified to have altered activity in at least one gram-positive organism said promoter being linked to at least one operator selected from the group consisting of xylO, tetO, trpO, malO and λc1O, wherein said at least one operator is positioned such that binding of at least one repressor to said at least one operator represses transcription from said fusion promoter;

(b) inhibiting the activity of said proliferation-required gene or gene product in said test cell by inducing transcription from said fusion promoter;

(c) contacting said test cell with a compound known to inhibit growth or proliferation of a cell, wherein the biological pathway on which said compound acts is known; and

(d) determining the degree to which said compound inhibits proliferation of said test cell relative to a cell which has not been contacted with said compound.

118. A method for identifying the biological pathway in which a proliferation-required gene or its gene product lies said method comprising:

(a) introducing into a test cell a construct comprising an inducible fusion promoter operably linked to a nucleic acid that is complementary to at least a portion of a proliferation-required gene, said fusion promoter comprising at least one promoter selected from the group consisting of T5, CP25, P32, P59, P1P2 and PL, said promoter being linked to at least one operator selected from the group consisting of xylO, tetO, trpO, malO and λc1O, wherein said at least one operator is positioned such that binding of at least one repressor to said at least one operator represses transcription from said fusion promoter;

(b) inhibiting the activity of said proliferation-required gene or gene product in said test cell by inducing transcription from said fusion promoter;

(c) contacting said test cell with a compound known to inhibit growth or proliferation of a cell, wherein the biological pathway on which said compound acts is known; and

(d) determining the degree to which said compound inhibits proliferation of said test cell relative to a cell which has not been contacted with said compound.

119. The method of claim 117, wherein said at least one operator is xylO.

120. The method of claim 119, wherein said at least one promoter is T5.

121. The method of claim 119, wherein said fusion promoter further comprises a second operator.

122. The method of claim 121, wherein said second operator is lacO.

123. The method of claim 117, wherein said inducer is xylose.

124. The method of claim 117, wherein said fusion promoter is titratable.

125. The method of claim 117, wherein said at least one gram-positive organism is selected from the group consisting of Bacillus anthracis, Clostridium botulinum, Clostridium difficile, Clostridium perfringens, Clostridium teteni, Corynebacterium diptheriae, Enterococcus faecalis, Enterococcus faecium, Lactococcus lactis, Listeria monocytogenes, Mycobacterium leprae, Mycobacterium tuberculosis, Nocardia asteroides, Staphylococcus aureus, Staphylococcus epidermidis, Staphylococcus xylosis, Streptococcus pneumoniae, Streptococcus mutans and any species falling within the genera of any of the above species.

126. The method of claim 117, wherein said at least one gram-positive organism is Staphylococcus aureus.

127. The method of claim 117, wherein said at least one gram-positive organism is Enterococcus faecalis.

128. A method for determining the biological pathway on which a test compound acts said method comprising:

(a) introducing into a cell a construct comprising an inducible fusion promoter operably linked to a nucleic acid that is complementary to at least a portion of a proliferation-required gene, wherein the biological pathway in which said gene or product of said gene lies is known, and wherein said fusion promoter comprises at least one promoter that is modified to have altered activity in at least one gram-positive organism said promoter being linked to at least one operator selected from the group consisting of xylO, tetO, trpO, malO and λc1O, wherein said at least one operator is positioned such that binding of at least one repressor to said at least one operator represses transcription from said fusion promoter;

(b) inhibiting the activity of said proliferation-required in said cell by inducing transcription from said fusion promoter;

(c) contacting said cell with a test compound; and

(d) determining the degree to which said test compound inhibits proliferation of said cell relative to a cell containing a which does not contain said construct.

129. A method for determining the biological pathway on which a test compound acts said method comprising:

(a) introducing into a cell a construct comprising an inducible fusion promoter operably linked to a nucleic acid that is complementary to at least a portion of a proliferation-required gene, wherein the biological pathway in which said gene or product of said gene lies is known, and wherein said fusion promoter comprises at least one promoter selected from the group consisting of T5, CP25, P32, P59, P1P2 and PL, said promoter being linked to at least one operator selected from the group consisting of xylO, tetO, trpO, malO and λc1O, wherein said at least one operator is positioned such that binding of at least one repressor to said at least one operator represses transcription from said fusion promoter;

(b) inhibiting the activity of said proliferation-required in said cell by inducing transcription from said fusion promoter;

(c) contacting said cell with a test compound; and

(d) determining the degree to which said test compound inhibits proliferation of said cell relative to a cell containing a which does not contain said construct.

130. The method of claim 128, wherein said at least one operator is xylO.

131. The method of claim 130, wherein said at least one promoter is T5.

132. The method of claim 130, wherein said fusion promoter further comprises a second operator.

133. The method of claim 132, wherein said second operator is lacO.

134. The method of claim 128, wherein said inducer is xylose.

135. The method of claim 128, wherein said fusion promoter is titratable.

136. The method of claim 128, wherein said at least one gram-positive organism is selected from the group consisting of Bacillus anthracis, Clostridium botulinum, Clostridium difficile, Clostridium perfringens, Clostridium teteni, Corynebacterium diptheriae, Enterococcus faecalis, Enterococcus faecium, Lactococcus lactis, Listeria monocytogenes, Mycobacterium leprae, Mycobacterium tuberculosis, Nocardia asteroides, Staphylococcus aureus, Staphylococcus epidermidis, Staphylococcus xylosis, Streptococcus pneumoniae, Streptococcus mutans and any species falling within the genera of any of the above species.

137. The method of claim 128, wherein said at least one gram-positive organism is Staphylococcus aureus.

138. The method of claim 128, wherein said at least one gram-positive organism is Enterococcus faecalis.

139. A method of manufacturing an antibiotic comprising the steps of:

(a) introducing into a cell a construct comprising an inducible fusion promoter operably linked to a nucleic acid that is complementary to at least a portion of a proliferation-required gene, said fusion promoter comprising at least one promoter that is modified to have altered activity in at least one gram-positive organism said promoter being linked to at least one operator selected from the group consisting of xylO, tetO, trpO, malO and λc1O, wherein said at least one operator is positioned such that binding of at least one repressor to said at least one operator represses transcription from said fusion promoter;

(b) sensitizing said cell by inducing transcription from said fusion promoter;

(c) contacting said sensitized cell with a compound;

(d) identifying a compound which substantially inhibits the proliferation of said sensitized cell relative to a cell which has not been sensitized; and

(e) manufacturing the compound so identified.

140. A method of manufacturing an antibiotic comprising the steps of:

(a) introducing into a cell a construct comprising an inducible fusion promoter operably linked to a nucleic acid that is complementary to at least a portion of a proliferation-required gene, said fusion promoter comprising at least one promoter selected from the group consisting of T5, CP25, P32, P59, P1P2 and PL, said promoter being linked to at least one operator selected from the group consisting of xylO, tetO, trpO, malO and λc1O, wherein said at least one operator is positioned such that binding of at least one repressor to said at least one operator represses transcription from said fusion promoter;

(b) sensitizing said cell by inducing transcription from said fusion promoter;

(c) contacting said sensitized cell with a compound;

(d) identifying a compound which substantially inhibits the proliferation of said sensitized cell relative to a cell which has not been sensitized; and

(e) manufacturing the compound so identified.

141. The method of claim 139, wherein said at least one operator is xylO.

142. The method of claim 141, wherein said at least one promoter is T5.

143. The method of claim 141, wherein said fusion promoter further comprises a second operator.

144. The method of claim 143, wherein said second operator is lacO.

145. The method of claim 139, wherein said inducer is xylose.

146. The method of claim 139, wherein said fusion promoter is titratable.

147. The method of claim 139, wherein said at least one gram-positive organism is selected from the group consisting of Bacillus anthracis, Clostridium botulinum, Clostridium difficile, Clostridium perfringens, Clostridium teteni, Corynebacterium diptheriae, Enterococcus faecalis, Enterococcus faecium, Lactococcus lactis, Listeria monocytogenes, Mycobacterium leprae, Mycobacterium tuberculosis, Nocardia asteroides, Staphylococcus aureus, Staphylococcus epidermidis, Staphylococcus xylosis, Streptococcus pneumoniae, Streptococcus mutans and any species falling within the genera of any of the above species.

148. The method of claim 139, wherein said at least one gram-positive organism is Staphylococcus aureus.

149. The method of claim 139, wherein said at least one gram-positive organism is Enterococcus faecalis.

150. A method for identifying a gene which is required for proliferation of a prokaryotic cell said method comprising the steps of:

(a) replacing the native promoter of a gene in the chromosome of a prokaryotic cell having an enhanced frequency of homologous recombination with a regulatable fusion promoter comprising at least one promoter that is modified to have altered activity in at least one gram-positive organism said promoter being linked to at least one operator selected from the group consisting of xylO, tetO, trpO, malO, lacO and λc1O, wherein said at least one operator is positioned such that binding of at least one repressor to said at least one operator represses transcription from said fusion promoter; and

(b) identifying cells in which the extent of proliferation of said cell when said fusion promoter is active at a first level is substantially different than the extent of proliferation of said cell when said fusion promoter is active at a second level, said first level being greater than said second level.

151. A method for identifying a gene which is required for proliferation of a prokaryotic cell said method comprising the steps of:

(a) replacing the native promoter of a gene in the chromosome of a prokaryotic cell having an enhanced frequency of homologous recombination with a regulatable fusion promoter comprising at least one promoter selected from the group consisting of T5, CP25, P32, P59, P1P2 and PL, said promoter being linked to at least one operator selected from the group consisting of xylO, tetO, trpO, malO, lacO and λc1O, wherein said at least one operator is positioned such that binding of at least one repressor to said at least one operator represses transcription from said fusion promoter; and

(b) identifying cells in which the extent of proliferation of said cell when said fusion promoter is active at a first level is substantially different than the extent of proliferation of said cell when said fusion promoter is active at a second level, said first level being greater than said second level.

152. The method of claim 150, wherein said fusion promoter is an inducible promoter.

153. The method of claim 150, wherein the step of replacing said native promoter comprises introducing a linear nucleic acid comprising a 5′ portion homologous to a first portion of said native promoter, a 3′ portion homologous to a second portion of said native promoter and said fusion promoter disposed between said 5′ portion and said 3′ portion into said cell such that homologous recombination occurs between said 5′ portion and said first portion of said native promoter and between said 3′ portion and said second portion of said native promoter.

154. The method of claim 153, wherein said linear nucleic acid is double stranded.

155. The method of claim 153, wherein said linear nucleic acid is single stranded.

156. A method for identifying a compound which inhibits the proliferation of a prokaryotic cell said method comprising the steps of:

(a) replacing the native promoter of a gene in the chromosome of a prokaryotic cell having an enhanced frequency of homologous recombination with a regulatable fusion promoter comprising at least one promoter that is modified to have altered activity in at least one gram-positive organism said promoter being linked to at least one operator selected from the group consisting of xylO, tetO, trpO, malO, lacO and λc1O, wherein said at least one operator is positioned such that binding of at least one repressor to said at least one operator represses transcription from said fusion promoter; and

(b) comparing the extent of proliferation of a first sample of said cell in the presence of said compound to the extent of proliferation of a second sample of said cell in the presence of said compound, wherein said first sample of said cell has a reduced activity of said fusion promoter activity relative the activity of said fusion promoter in said second sample of said cell and wherein said compound inhibits the proliferation of said cell if the extent of proliferation of said first sample of said cell is substantially less than the extent of proliferation of said second sample of said cell.

157. A method for identifying a compound which inhibits the proliferation of a prokaryotic cell said method comprising the steps of:

(a) replacing the native promoter of a gene in the chromosome of a prokaryotic cell having an enhanced frequency of homologous recombination with a regulatable fusion promoter comprising at least one promoter selected from the group consisting of T5, CP25, P32, P59, P1P2 and PL, said promoter being linked to at least one operator selected from the group consisting of xylO, tetO, trpO, malO, lacO and λc1O, wherein said at least one operator is positioned such that binding of at least one repressor to said at least one operator represses transcription from said fusion promoter; and

(b) comparing the extent of proliferation of a first sample of said cell in the presence of said compound to the extent of proliferation of a second sample of said cell in the presence of said compound, wherein said first sample of said cell has a reduced activity of said fusion promoter activity relative the activity of said fusion promoter in said second sample of said cell and wherein said compound inhibits the proliferation of said cell if the extent of proliferation of said first sample of said cell is substantially less than the extent of proliferation of said second sample of said cell.

158. The method of claim 156, wherein the step of replacing said native promoter comprises introducing a linear nucleic acid comprising a 5′ portion homologous to a first portion of said native promoter, a 3′ portion homologous to a second portion of said native promoter and said fusion promoter disposed between said 5′ portion and said 3′ portion into said cell such that homologous recombination occurs between said 5′ portion and said first portion of said native promoter and between said 3′ portion and said second portion of said native promoter.

159. The method of claim 158, wherein said linear nucleic acid is double stranded.

160. The method of claim 158, wherein said linear nucleic acid is single stranded.

161. A method for identifying a gene which is required for proliferation of a prokaryotic cell said method comprising the steps of:

(a) introducing at least one operator selected from the group consisting of xylO, tetO, trpO, malO, lacO and λc1O into a prokaryotic cell having an enhanced frequency of homologous recombination such that said at least one operator regulates transcription of a target nucleic acid in the chromosome of said cell; and

(b) identifying cells in which the extent of proliferation of said cell when said target nucleic acid is transcribed at a first level is substantially different than the extent of proliferation of said cell when said target nucleic acid is transcribed at a second level, said first level being greater than said second level.

162. The method of claim 161, wherein the step of introducing said at least one operator comprises introducing a linear nucleic acid comprising a 5′ portion homologous to a first portion of the chromosome of said cell, a 3′ portion homologous to a second portion of the chromosome of said cell and said at least one operator disposed between said 5′ portion and said 3′ portion into said cell such that homologous recombination occurs between the 5′ portion and said first portion of the chromosome of said cell and between the 3′ portion and said second portion of the chromosome of said cell.

163. The method of claim 162, wherein said linear nucleic acid is double stranded.

164. The method of claim 162, wherein said linear nucleic acid is single stranded.

165. A method of identifying a compound which inhibits the proliferation of a prokaryotic cell said method comprising the steps of:

(a) obtaining a prokaryotic cell in which transcription of a nucleic acid required for proliferation of said cell is regulated by at least one operator which has been introduced into the chromosome of said cell said at least one operator selected from the group consisting of xylO, tetO, trpO, malO, lacO and λc1O;

(b) sensitizing said cell by growing said cell under conditions in which the level of transcription of said gene is lower than that of a wild type cell;

(c) contacting said sensitized cell with said compound; and

(d) determining the degree to which said compound inhibits the growth of said sensitized cell relative to an unsensitized cell.

166. The method of claim 165, wherein said cell has an enhanced frequency of homologous recombination.

167. The method of claim 165, wherein said linear nucleic acid is single stranded.

168. The method of claim 165, wherein said linear nucleic acid is double stranded.

169. A method of identifying a nucleic acid sequence having promoter activity in Enterococcus faecalis said method comprising the steps of:

(a) inserting a candidate nucleic acid into a vector comprising lacL-lacM reporter genes such that said candidate nucleic acid is upstream of the lacL-lacM reporter genes;

(b) introducing said vector comprising said candidate nucleic acid into Enterococcus faecalis;

(c) detecting expression of the lacL-lacM reporter genes, wherein expression of said lacL-lacM reporter genes indicates that said candidate nucleic acid sequence has promoter activity.

170. The method of claim 169, wherein said detecting step comprises measuring β-galactosidase activity.

171. The method of claim 169, wherein said candidate nucleic acid is a promoter modified to increase activity in a gram-positive organism.

172. The method of claim 169, wherein said lacL-lacM reporter gene is derived from Leuconostoc mesenteroides.

173. The method of claim 169, wherein said vector is pEPEF1.

### Definitions

The following definitions are provided so as to facilitate the understanding of the invention as set out herein.

By “activity against a gene product” is meant having the ability to inhibit the function or to reduce the level or activity of the gene product in a cell. This includes, but is not limited to, inhibiting the enzymatic activity of the gene product or the ability of the gene product to interact with other biological molecules required for its activity, including inhibiting the gene product's assembly into a multimeric structure.

By “activity against a protein” is meant having the ability to inhibit the function or to reduce the level or activity of the protein in a cell. This includes, but is not limited to, inhibiting the enzymatic activity of the protein or the ability of the protein to interact with other biological molecules required for its activity, including inhibiting the protein's assembly into a multimeric structure.

By “activity against a nucleic acid” is meant having the ability to inhibit the function or to reduce the level or activity of the nucleic acid in a cell. This includes, but is not limited to, inhibiting the ability of the nucleic acid interact with other biological molecules required for its activity, including inhibiting the nucleic acid's assembly into a multimeric structure.

By “activity against a gene” is meant having the ability to inhibit the function or expression of the gene in a cell. This includes, but is not limited to, inhibiting the ability of the gene to interact with other biological molecules required for its activity.

By “activity against an operon” is meant having the ability to inhibit the function or reduce the level of one or more products of the operon in a cell. This includes, but is not limited to, inhibiting the enzymatic activity of one or more products of the operon or the ability of one or more products of the operon to interact with other biological molecules required for its activity.

By “antibiotic” is meant an agent which inhibits the proliferation of a cell or microorganism.

The term “aptamer” as used herein refers to a single-stranded or double-stranded oligodeoxyribonucleotide, oligoribonucleotide or modified derivatives of oligodeoxyribonucleotides or oligoribonucleotides that specifically binds and alters the biological function of a target molecule, which can be a protein, peptide and derivatives thereof.

By “biological pathway” is meant any discrete cell function or process that is carried out by a gene product or a subset of gene products. Biological pathways include anabolic, catabolic, enzymatic, biochemical and metabolic pathways as well as pathways involved in the production of cellular structures such as cell walls. Biological pathways that are usually required for proliferation of cells or microorganisms include, but are not limited to, cell division, DNA synthesis and replication, RNA synthesis (transcription), protein synthesis (translation), protein processing, protein transport, fatty acid biosynthesis, electron transport chains, cell wall synthesis, cell membrane production, synthesis and maintenance, and the like.

The term “expression” is defined as the production of a sense or antisense RNA molecule from a gene, gene fragment, genomic fragment, chromosome, operon or portion thereof. Expression can also be used to refer to the process of peptide or polypeptide synthesis.

An “expression vector” is defined as a vehicle by which a ribonucleic acid (RNA) is transcribed from a nucleic acid carried within the expression vehicle. The expression vector can also contain features that permit translation of a protein product from the transcribed RNA message expressed from the exogenous nucleic acid carried by the expression vector. Accordingly, an expression vector can produce an RNA molecule as its sole product or the expression vector can produce a RNA molecule that is ultimately translated into a protein product.

By “identifying a compound” is meant to screen one or more compounds in a collection of compounds such as a combinatorial chemical library, a natural product library or other library of chemical compounds or to characterize a single compound by testing the compound in a given assay and determining whether it exhibits the desired activity.

By “inducer” is meant an agent, solution or environmental condition which, when placed in contact with a cell or microorganism, increases transcription, or inhibitor and/or promoter clearance/fidelity, from a desired promoter.

By “inhibit activity of a gene or gene product” is meant having the ability to interfere with the function of a gene or gene product in such a way as to decrease expression of the gene, in such a way as to reduce the level or activity of a product of the gene or in such a way as to inhibit the interaction of the gene or gene product with other biological molecules required for its activity. Agents which inhibit the activity of a gene include agents that inhibit transcription of the gene, agents that inhibit processing of the transcript of the gene, agents that reduce the stability of the transcript of the gene, and agents that inhibit translation of the mRNA transcribed from the gene. In microorganisms, agents which inhibit the activity of a gene can act to decrease expression of the operon in which the gene resides or alter the folding or processing of operon RNA so as to reduce the level or activity of the gene product. The gene product can be a non-translated RNA such as ribosomal RNA, a translated RNA (mRNA) or the protein product resulting from translation of the gene mRNA. Of particular utility to the present invention are antisense RNAs that have activities against the operons or genes to which they specifically hybridize.

As used herein, “lac operator”, “lac operator sequence”, or “laco” is intended to encompass the operator having SED ID NO.: 11 and any other lac operator sequences consistent with the intended uses described herein.

As used herein, “λc1 operator”, “λc1 operator sequence”, or “λc1O” is intended to encompass all lambda operators to which the λc1 repressor or variants thereof are capable of binding. Such operators include, but are not limited to, λO_{R1} (SEQ ID NO.: 54), λO_{R2} (SEQ ID NO.: 55), λO_{R3} (SEQ ID NO.: 56), λO_{L1} (SEQ ID NO.: 57), λO_{L2} (SEQ ID NO.: 58), λO_{L3} (SEQ ID NO.: 59).

As used herein, “mal operator”, “mal operator sequence” or “malO” is intended to encompass the two mal operators MalMop (SEQ ID NO.: 60) and MalXop (SEQ ID NO.: 61), as described in Nieto et al., J. Biol. Chem. 272,30860-30865 (1997), the disclosure of which is incorporated herein by reference in its entirety, and any other mal operator sequences consistent with the intended uses described herein.

As used herein, “nucleic acid” means DNA, RNA, or modified nucleic acids. Thus, the terminology “the nucleic acid of SEQ ID NO: X” or “the nucleic acid comprising the nucleotide sequence” includes both the DNA sequence of SEQ ID NO: X, the complement thereof, and an RNA sequence in which the thymidines in the DNA sequence have been substituted with uridines in the RNA sequence and in which the deoxyribose backbone of the DNA sequence has been substituted with a ribose backbone in the RNA sequence.

As used herein, “polynucleotide” has the same meaning as nucleic acid.

As used herein, “proliferation-inhibiting” encompasses instances where the absence or substantial reduction of a gene transcript and/or gene product completely eliminates cell growth as well as instances where the absence of a gene transcript and/or gene product merely reduces cell growth. A proliferation-inhibiting antisense nucleic acid is one that can cause a reduction of a gene transcript and/or gene product that is sufficient to reduce or eliminate the growth or viability of the cell or microorganism.

As used herein, “proliferation-required” or “required for proliferation” encompasses instances where the absence or substantial reduction of a gene transcript and/or gene product completely eliminates cell growth as well as instances where the absence of a gene transcript and/or gene product merely reduces cell growth. A proliferation-required gene or gene family is one where, in the absence or substantial reduction of a gene transcript and/or gene product, growth or viability of the cell or microorganism is reduced or eliminated.

As used herein, “sub-lethal” means a concentration of an agent below the concentration required to inhibit all cell growth.

As used herein, “tetracycline analog” or “Tc analog” is intended to include compounds which are structurally related to tetracycline and which bind to the Tet repressor with a Kₐ of at least about 10⁻⁶ M. Preferably, the tetracycline analog binds with an affinity of about 10⁻⁹ M or greater. Examples of such tetracycline analogs include, but are not limited to, anhydrotetracycline (atc), doxycycline, chlorotetracycline, oxytetracycline and others disclosed by Hlavka and Boothe, “The Tetracyclines,” in Handbook of Experimental Pharmacology 78, R. K. Blackwood et al. (eds.), Springer-Verlag, Berlin, N.Y., 1985; L. A. Mitscher, “The Chemistry of the Tetracycline Antibiotics”, Medicinal Research 9, Dekker, N.Y., 1978; Noyee Development Corporation, “Tetracycline Manufacturing Processes” Chemical Process Reviews, Park Ridge, N.J., 2 volumes, 1969; R. C. Evans, “The Technology of the Tetracyclines”, Biochemical Reference Series 1, Quadrangle Press, New York, 1968; and H. F. Dowling, “Tetracycline”, Antibiotic Monographs, no. 3, Medical Encyclopedia, New York, 1955. Preferred Tc analog for high-level stimulation of transcription is anhydrotetracycline. For use in prokaryotic organisms, a Tc analog can be chosen which has reduced antibiotic activity compared to Tc. Examples of such Tc analogues are anhydrotetracycline, epioxytetracycline and cyanotetracycline.

As used herein, “Tet repressor” is intended to describe a protein occurring in nature which represses transcription from tet operator sequences in prokaryotic cells in the absence of Tc or Tc analog. Tetracycline repressors of the present invention may be obtained from any class of tetracycline repressor (e.g., TetR(A), TetR(B), TetR(C), TetR(D), TetR(E), TetR(G), TetR(H), TetR(J), TetR(Z), or combinations thereof). The nucleotide sequences of Tet repressors of representative members of the A, B, C, D and E classes are known (e.g., see Waters, S. H. et al. (1983) Nucl. Acids Res 11:6089-6105, Hillen, W. and Schollmeier, K. (1983) Nucl. Acids Res. 11:525-539 and Postle, K. et al. (1984) Nucl. Acids Res. 12:4849-4863, Unger, B. et al. (1984) Gene 31: 103-108, Unger, B. et al. (1984) Nucl Acids Res. 12:7693-7703 and Tovar, K. et al. (1988) Mol. Gen. Genet. 215:76-80, respectively).

As used herein, “tet operator”, “tet operator sequence” or “tetO” is intended to encompass all classes of tet operator sequences, e.g. (e.g., TetO(A) (SEQ ID NO.: 62), TetO(B) (SEQ ID NO.: 63), TetO(C) (SEQ ID NO.: 64), TetO(D) (SEQ ID NO.: 65), TetO(E) (SEQ ID NO.: 66), TetO(G), TetO(H), TetO(J), and TetO(Z). Nucleotide sequences of these five classes of tet operators are described in Waters, S. H. et al. (1983) cited supra, Hillen, W. and Schollenmeier, K. (1983) cited supra, Stuber, D. and Bujard, H. (1981) Proc. Natl. Acad. Sci. USA 78:167-171, Unger, B. et al. (1984) cited supra and Tovar, K. et al. (1988) cited supra.

As used herein, “trp operator”, “trp operator sequence” or “trpO” is intended to encompass all classes of trp operator sequences consistent with the intended uses described herein. One example is the trp operator of SEQ ID NO.: 67.

As used herein, “xyl operator”, “xyl operator sequence” or “xylO” is intended to encompass all classes of xyl operator sequences consistent with the intended uses described herein. One example is the xyl operator of SEQ ID NO.: 68.

### BRIEF DESCRIPTION OF THE DRAWINGS

FIG. 1 is a sequence map showing the Xyl-T5 fusion promoter (SEQ ID NO.: 1) showing the following elements: (1) −45 AT Box; (2) −35 RNA polymerase recognition site; (3) a 17 bp intervening sequence that can be replaced by a second operator; (4) −10 RNA polymerase recognition site; (5) the xyl operator; (6) multiple cloning sequence; (7A) first rrnB terminator; and (7B) second rrnB terminator.

FIG. 2 is a sequence map showing the Xyl-CP25 fusion promoter (SEQ ID NO.: 2) showing the following elements: (1) a portion of the xylR gene; (2) −35 RNA polymerase recognition site; (3) a 17 bp intervening sequence that can be replaced by a second operator; (4) −10 RNA polymerase recognition site; and (5) the xyl operator. Also shown are recognition sites for the restriction enzymes (a) RsrII and (b) XhoI.

FIG. 3A illustrates one embodiment of the present promoter replacement method in which a linear double stranded promoter replacement construct comprising a nucleic acid encoding a selectable marker, a transcriptional terminator, and a regulatable fusion promoter is inserted into the chromosome of a cell expressing the RecE and RecT gene products of the Rac prophage or gene products having analogous functions.

FIG. 3B illustrates one embodiment of the present promoter replacement method in which a linear single stranded promoter replacement construct comprising a 5′ portion homologous to a target sequence, a 3′ portion homologous to a target sequence and a regulatable fusion promoter is inserted into the chromosome of a cell expressing the λ Beta and Gam proteins.

FIG. 4A illustrates one embodiment of the operator insertion method in which a linear double stranded regulatory element insertion construct comprising a nucleic acid encoding a selectable marker, a transcriptional terminator, and an operator is inserted into the chromosome of cells expressing the RecE and RecT gene products of the Rac prophage or gene products having analogous functions.

FIG. 4B illustrates one embodiment of the operator insertion method in which a linear single stranded regulatory element insertion construct comprising a 5′ portion homologous to a target sequence, a 3′ portion homologous to a target sequence and an operator is inserted into the chromosome of a cell expressing the λ Beta and Gam proteins.

FIG. 5 shows a plasmid map of the pXyl-T5 P15a Staphylococcus expression vector.

FIG. 6 shows a plasmid map for the pEPEF-X series of Enterococcus expression vectors.

FIG. 7 shows a bar graph depicting the ratio of RNA measured from xylose induced cells versus non-induced cells. RNA measurements were made in real time by RT-PCR and the samples were normalized for loading by using parallel 16S rRNA controls. Samples were prepared and analyzed in duplicate and the averages are plotted.

FIG. 8 shows a bar graph comparing the induced and uninduced activities of the Xyl-CP25, Xyl-P32, Xyl-P59 and Xyl-P1P2 fusion promoters in Enterococcus faecalis. The activity of each fusion promoter was determined by measuring the β-galactosidase activity produced by the expression of lacL-lacM reporter genes operably linked downstream of each fusion promoter.

FIG. 9 shows a bar graph comparing the induced and uninduced activities of the Xyl-T5 and Xyl-CP25 fusion promoters in Enterococcus faecalis. A comparison of the induced and uninduced activities of the Xyl-T5 fusion promoter in Staphylococcus aureus and Enterococcus faecalis is also shown. The activity of each fusion promoter was determined by measuring the β-galactosidase activity produced by the expression of lacL-lacM reporter genes operably linked downstream of each fusion promoter.

FIG. 10 shows a bar graph depicting the activity of the Xyl-P59 fusion promoter over a range of xylose concentrations from 0 to 10%. The activity of Xyl-P59 was determined by measuring the β-galactosidase activity produced by the expression of lacL-lacM reporter genes operably linked downstream of this fusion promoter.

FIG. 11 shows a bar graph comparing the induced and uninduced activities of the Xyl-P59, Xyl-PL, and modified Xyl-PL (Xyl-PL10) fusion promoters in Enterococcus faecalis. The activity of each fusion promoter was determined by measuring the β-galactosidase activity produced by the expression of lacL-lacM reporter genes operably linked downstream of each fusion promoter.

FIG. 12 is an IPTG dose response curve in E. coli transformed with an IPTG-inducible plasmid containing either an antisense clone to the E. coli ribosomal protein rplW (AS-rplW) which is required for protein synthesis and essential for cell proliferation, or an antisense clone to the elaD (AS-elaD) gene which is not known to be involved in protein synthesis and which is also essential for proliferation.

FIG. 13A is a tetracycline dose response curve in E. coli transformed with an IPTG-inducible plasmid containing antisense to rplW (AS-rplW) in the absence (0) or presence of IPTG at concentrations that result in 20% and 50% growth inhibition.

FIG. 13B is a tetracycline dose response curve in E. coli transformed with an IPTG-inducible plasmid containing antisense to elaD (AS-elaD)in the absence (0) or presence of IPTG at concentrations that result in 20% and 50% growth inhibition.

FIG. 14 is a graph showing the fold increase in tetracycline sensitivity of E. coli transfected with antisense clones to essential ribosomal proteins L23 (AS-rplW) and L7/L12 and L10 (AS-rplLrplJ). Antisense clones to genes known to not be directly involved in protein synthesis, atpB/E (AS-atpB/E ), visC (AS-visC), elaD (AS-elaD), yohH (AS-yohH), are much less sensitive to tetracycline.

FIG. 15 illustrates the results of an assay in which Staphylococcus aureus cells expressing an antisense nucleic acid complementary to the gyrB gene encoding the β subunit of gyrase were contacted with several antibiotics whose targets were known.

### DETAILED DESCRIPTION OF THE INVENTION

The invention described herein involves the construction and use of fusion promoters that can be tightly and/or finely regulated (e.g., with a xyl, tet, mal, lac, trp, λc1, or other suitable operators), constructs and cells having these promoters, methods of expression of RNAs, peptides and proteins, methods to identify genes essential for the growth of bacteria, and methods to identify compounds that inhibit bacterial growth.

Some embodiments of this invention contemplate the preparation of several different bacterial fusion promoters which contain at least one operator associated with a promoter sequence. The at least one operator is positioned such that the binding of at least one repressor to the operator represses transcription from the promoter. The promoter sequence may be a wildtype promoter obtained from a bacterium, virus or other microorganism or it may be a promoter sequence that has been modified to have altered activity in Gram-positive organisms. One embodiment, for example, is a fusion promoter comprising a promoter sequence modified to have altered activity in at least one Gram-positive organism linked to one or more operators including, but not limited to, the xylose operator (“xylO”), the tetracycline operator (“tetO”), the maltose operator (“malO”), lactose operator (“lacO”), the tryptophan operator (“trpO”), and the lambda Cl operator (“λc1O”). These operators can be induced by the following inducers, respectively: xylose or analog thereof, tetracycline or analog thereof, maltose or analog thereof, lactose or analog thereof, tryptophan or analog thereof, and temperature.

In some embodiments, fusion promoters based on the bacteriophage T5 promoter and the xylose operator are contemplated. The T5 promoter has been shown to be capable of directing the efficient expression of genes primarily in Escherichia coli. (See e.g., Gentz et al, Proc. Natl. Acad. Sci. USA 78:4936-4940 (1981); Rommens et al., Nucleic Acids Res. 11:5921-5940 (1983); Wang et al., Gene 242:105-114 (2000); and U.S. Pat. Nos. 4,495,280 and 4,689,406) and the xylose operator/repressor system has been shown to regulate expression in some Gram-positive bacteria. (See e.g., Dahl et al., J. Mol. Biol. 243:413-424 (1994); Sizemore et al., J. Bacteriol. 174:3042-3048 (1992); Wieland et al., Gene 158:91-96 (1995); Kreuzer et al., J. Bacteriol. 171:3840-3845 (1989); Lokinan et al., J. Bacteriol. 179:5391-5397 (1997); and Erbeznik et al., J. Bacteriol. 180:1103-1109 (1998)).

In a preferred embodiment, xylO is operably linked to a T5 promoter such that transcription from the fusion promoter is inducible by agents that inhibit the binding of the xylose repressor (encoded by xylR) to the xylose operator (xylO). The xylO can be the xylO from S. xylosis or B. subtilis or a number of other bacteria. This embodiment, referred to as the Xyl-T5 promoter, can also contain several other elements including, but not limited to, AT-rich boxes (AT Box) that enhance the efficiency of gene expression in Gram-positive bacteria. The use of an AT-Box-enchanced T5 promoter has been described by D. J. Henner in “Expression of Heterologous Genes in Bacillus subtilis” Methods Enzymol., 195:199 (1990), the disclosure of which is incorporated herein by reference in its entirety.

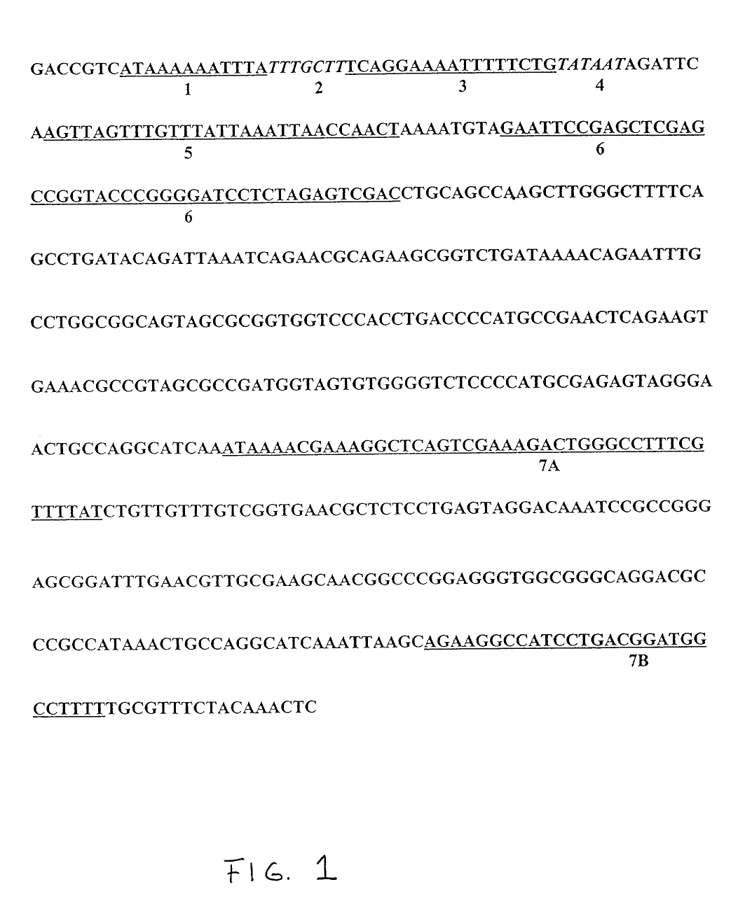

In some embodiments fusion promoters containing one or more AT Boxes are contemplated. The AT Box is positioned such that it increases the level of transcription from the fusion promoter in Gram-positive bacteria. For the Xyl-T5 embodiment, a consensus −45 AT Box is shown in FIG. 1 at position (1). Many variations of the AT Box can be used. For example, the AT Box can have more or less adenine or thymine nucleotides or different sequences or combinations of adenine and thymine molecules. In this respect, the diversity of the AT box is limited only by the ability of this AT rich region to facilitate transcription in Gram-positive bacteria. In other embodiments of the present invention, fusion promoters constructed from T5 promoter derivatives which have been modified to have altered activity in Gram-positive organisms are contemplated.

A preferred embodiment of the Xyl-T5 fusion promoter is shown in FIG. 1. In italics, at positions (2) and (4) of FIG. 1 are shown the −35 and −10 RNA polymerase recognition sites (−35 Box and −10 Box), respectively. The 17 nucleotide long element shown at position (3) is a site in which a second operator can be inserted if desired. The second operator is positioned such that the binding of a repressor to the second operator represses transcription from the promoter. In some embodiments, the second operator may be the xylO, tetO, malO, lacO, trpO, or λc1O. At position (5) is shown the xyl operator (xylO), which is placed just downstream of the −10 Box. The distance between xylO and the T5 promoter can be made shorter or longer provided the ability to sustain repression of the T5 promoter is maintained. In some embodiments, a multiple cloning sequence (designated at position (6)) is also inserted downstream of the xyl operator to allow nucleic acids to be transcribed from the promoter when operably linked to the promoter. Many different multiple cloning sequences can be inserted at position (6) and the multiple cloning sequence shown is only one example. Additionally, some embodiments can include a terminator sequence, to facilitate termination of the RNA transcribed from the promoter at a desired position (e.g., the first rrnB terminator shown at position (7A) and/or the second rrnB terminator shown at position (7B)). Preferably, the terminator sequence allows for Rho independent transcription termination.

The present invention also contemplates fusion promoters based on promoter sequences discovered in Lactococcus lactis linked to at least one operator. For example, the Lactococcus lactis promoters CP25 (Jensen and Hammer, Appl. Environ. Microbiol. 64:82-87 (1998), the disclosure of which is incorporated herein by reference in its entirety), P32 and P59 (Bruberg et al., Appl. Microbiol. Biotechnol. 42:108-115 (1994), the disclosure of which is incorporated herein by reference in its entirety), P1P2 (Nilsson and Johansen, Biochimica Biophysica Acta. 1219:141-144 (1994), the disclosure of which is incorporated herein by reference in its entirety), and PL (Madsen et al., Appl. Environ. Microbiol. 67:1128-1139, the disclosure of which is incorporated herein by reference in its entirety) can be fused to to at least one operator. In some embodiments of the present invention, fusion promoters constructed from derivatives of the above Lactococcus lactis sequences which have been modified to have altered activity in Gram-positive organisms are contemplated.

It will be appreciated that a regulatable fusion promoter suite can be constructed which provides a wide dynamic range of both basal and maximal transcriptional activities. Each fusion promoter within this suite can be tightly and finely regulated over its own characteristic range of transcriptional activity. Furthermore, the regulatable fusion promoters within the suite can comprise different promoters operably linked to the same operators thus providing for a common means of regulation of the promoters within the suite. In some embodiments, the promoters within the set of regulatable fusion promoters within the suite can be linked to at least one operator contained in a promoterless expression vector that is capable of replicating in one or more Gram-positive organisms. Accordingly, a regulatable fusion promoter having an appropriate basal level and/or maximal level of transcriptional activity can be selected from the suite for use in the appropriate application.

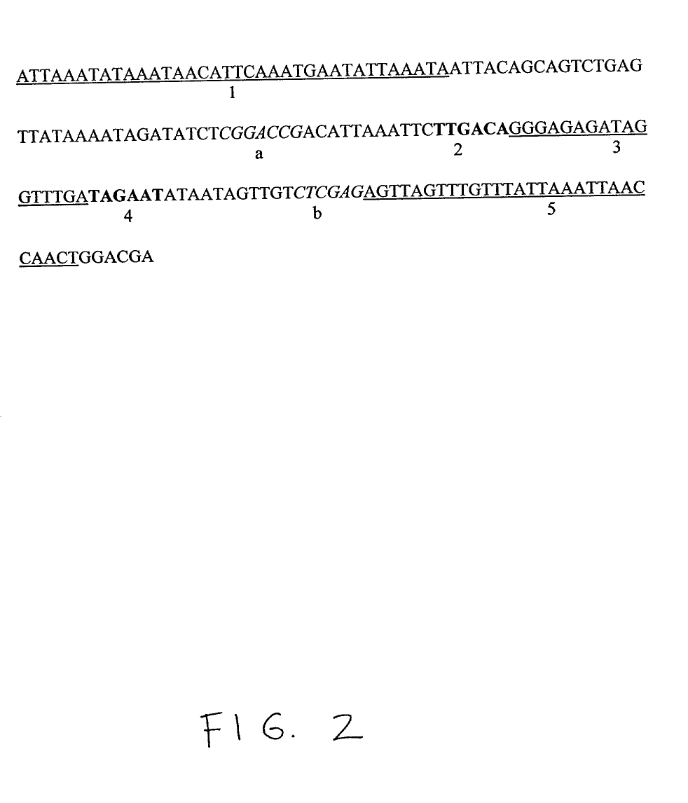

In some embodiments, fusion promoters based on promoter sequences discovered in Lactococcus actively promote transciption in Enterococcus faecalis and other Gram-positive organisms. One embodiment is the Xyl-CP25 fusion promoter shown in FIG. 2. Position (1) of FIG. 2 shows a portion of the xylR gene located upstream from the fusion promoter. Positions (2) and (4) show the −35 and −10 Boxes of the CP25 promoter sequence, respectively. The 17 nucleotide long element shown at position (3) is a site in which a second operator can be inserted if desired. The second operator is positioned such that the binding of a repressor to the second operator represses transcription from the promoter. In some embodiments, the second operator may be the xylO, tetO, malO, lacO, trpO, or λc1O operator. At position (5) is shown the xyl operator (xylO), which is placed just downstream of the −10 Box. The distance between xylO and the CP25 promoter can be made shorter or longer provided the ability to sustain repression of the CP25 promoter is maintained. The positions of unique RsrII and XhoI recognition sites flanking the promoter are indicated as (a) and (b), respectively. In some embodiments, the RsrII and XhoI sites can be used to exchange the CP25 promoter with other promoter sequences, such as promoters selected from SEQ ID NOs.: 36-45.

In other embodiments of the fusion promoters described herein, elements such as multiple cloning sites (MCS) and transcription terminators can be linked downstream. Preferably, the terminator sequence allows for Rho independent transcription termination. For example the rrnB terminator (See e.g., Orosz et al., Eur. J. Biochem., 201:653-659 (1991), the disclosure of which is incorporated by reference in its entirety) may be utilized.

Some fusion promoters of the present invention function in Staphylcoccus aureus and Enterococcus faecalis. Other fusion promoters described herein function in Gram-positive organisms such as Bacillus anthracis, Clostridium botulinum, Clostridium difficile, Clostridium perfringens, Clostridium teteni, Corynebacterium diptheriae, Enterococcus faecalis, Enterococcus faecium, Lactococcus lactis, Listeria monocytogenes, Mycobacterium leprae, Mycobacterium tuberculosis, Nocardia asteroides, Staphylococcus aureus, Staphylococcus epidermidis, Staphylococcus xylosis, Streptococcus pneumoniae, Streptococcus mutans or any species falling within the genera of any of the above species.

In another aspect of the invention, two or more operators are linked to a promoter. The operators are positioned such that binding of repressors to the operators represses transcription from the promoter. The operators may be identical or different. By controlling gene expression with multiple operators (e.g., using a plurality of operators that can be selectively modulated in specific organisms), genetic information can be safely transferred between bacterial species and expression can be selectively induced and regulated. For example, repressors that bind to one or more of the operators may be expressed in a first bacterial species or strain but not in a second species. Advantageously, some embodiments maintain tight repression of a potentially lethal transcript in a first species or strain that is used to amplify the construct but the repression can be efficiently removed in a second species or strain in which transcription from the promoter is desired.

One embodiment, for example, concerns a fusion promoter that selectively enables the repression of transcription in Escherichia coli and Staphylococcus species. The “Xyl/Lac-T5” promoter comprises the lacO operably linked to the Xyl-T5 promoter so that transcription of the genetic information associated with the promoter can be induced in Staphylococcus by adding xylose or an analog thereof and/or in Escherichia coli by adding lactose or an analog thereof (e.g., IPTG). This selective regulation may be a consequence of the fact that Staphylococcus does not express the lac repressor that binds to the lacO while Escherichia coli does not express the xylR that binds to the xylO. In this manner, the construct can be efficiently amplified in Escherichia coli and retain the ability to express the desired transcript in Staphylococcus once the binding of xylR to xylO is inhibited by adding an inducer such as xylose or an analog thereof.

Similar multioperator constructs, which incorporate both lacO and xylO so as to provide regulatable expression in both Escherichia coli and Gram-positive organisms, can be made using promoter sequences described herein, including promoters selected from SEQ ID NOs.: 26-35.

By following the approaches described in the examples below, with modifications as would be apparent to one of skill in the art, the preparation of a fusion promoter comprising operators other than those described in the examples can be accomplished without undue burden or extensive experimentation.

The present invention also contemplates fusion promoters that have been modified to have altered activity in Gram-positive organisms. Modifications to fusion promoters can be made, for example, by synthesizing the modified DNA sequences, PCR based mutagenesis, site-directed mutagenesis techniques, DNA shuffling or any other techniques for making site specific mutations.

In some embodiments of the present invention, nucleotides in the −10, −35 or −45 Boxes of a promoter are changed to make those sequence elements look more or less like the consensus −10, −35 or −45 Box sequences in the desired Gram-positive organisms. Such modification can have the affect of either increasing or decreasing the activity of the fusion promoter.

It will be appreciated that a regulatable fusion promoter suite can be constructed which provides a wide dynamic range of both basal and maximal transcriptional activities. It will also be appreciated that modifying a regulatable fusion promoter so as to increase or decrease its activity can be used to expand the range of both basal and maximal transcriptional activities of a suite of regulatable fusion promoters. Each modified fusion promoter within this suite can be tightly and finely regulated over its own characteristic range of transcriptional activity. Furthermore, the modified regulatable fusion promoters within the suite can comprise different promoters operably linked to the same operators thus providing for a common means of regulation of the promoters within the suite. In some embodiments, the promoter within the set of modified regulatable fusion promoters within the suite can be linked to at least one operator contained in a promoterless expression vector that is capable of replicating in one or more Gram-positive organisms. Accordingly, a regulatable fusion promoter having an appropriate basal level and/or maximal level of transcriptional activity can be selected from the suite for use in the appropriate application.

In other embodiments, promoters are modified by altering the length of the region between the operator or transcription start site and the −10 Box, altering the length and/or composition of the region between the −10 Box and the −35 Box, and altering the length and/or composition of the AT rich region upstream of the −35 Box (i.e. the −45 Box). It will also be appreciated that modifications which affect promoter activity can include addition of elements such as the CRE region for catabolite repression in Gram-positive bacteria. These additional elements can be further modified so that they are more or less like their corresponding consensus sequence.

In some embodiments, modified fusion promoters can function in one or more Gram-positive organisms. For example, some modified fusion promoters of the present invention function in Staphycoccus aueus and Enterococcus faecalis. Other modified fusion promoters described herein function in Gram-positive organisms such as Bacillus anthracis, Clostridium botulinum, Clostridium difficile, Clostridium perfringens, Clostridium teteni, Corynebacterium diptheriae, Enterococcus faecalis, Enterococcus faecium, Lactococcus lactis, Listeria monocytogenes, Mycobacterium leprae, Mycobacterium tuberculosis, Nocardia asteroides, Staphylococcus aureus, Staphylococcus epidermidis, Staphylococcus xylosis, Streptococcus pneumoniae, Streptococcus mutans or any species falling within the genera of any of the above species.

In some embodiments of the present invention, the promoters described herein are used to initiate transcription of an RNA (sense or anti-sense) in bacteria, desirably Gram-positive bacteria. In another embodiment, random polynucleotides (e.g., nucleic acids generated by oligonucleotide synthesis, shearing of bacterial genomic DNA or enzymatic digestion, such as digestion with restriction endonucleases, DNase I or other nucleases) are operably linked to a promoter described herein. These constructs are transferred to bacteria and can be used to transcribe RNA.

In one embodiment of the present invention, a fusion promoter comprising an operator, such as xylO, and a promoter, such one selected from SEQ ID NOs.: 36-45, is operably linked to a nucleic acid to be transcribed from the promoter, the construct is introduced into bacteria, a solution of xylose or an analog thereof is provided (e.g., a 2.0% xylose solution), and induction of transcription proceeds. Preferred nucleic acids that are operably linked to the fusion promoter are at least 6, at least 10, at least 15, at least 20, at least 100, at least 200, at least 300, at least 400, at least 500, at least 800 or more than 800 nucleotides in length and, in some embodiments, the nucleic acids that are operably linked to the fusion promoter encode a transcript that is complementary to at least 6, at least 10, at least 15, at least 20, at least 100, at least 200, at least 300, at least 400, at least 500, at least 800 or more than 800 nucleotides within the genome of a Gram-positive organism, such as Staphylococcus or Enterococcus, which code for a gene product such as a protein or RNA. That is, nucleic acids that are operably linked to the fusion promoter can be in the “sense” or “antisense” orientation and can be at least 6, 7, 8, 9, 10, 11, 12, 13, 14, 15, 16, 17, 18, 19, 20, 20, 21, 22, 23, 24, 25, 26, 27, 28, 29, 30, 31, 32, 33, 34, 35, 36, 37, 38, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 250, 300, 350, 400, 450, 500, 550, 600, 650, 700, 750, 800 or more than 800 nucleotides in length.

In another embodiment, the promoters described herein are used in an expression construct to initiate the production of a polypeptide or protein in bacteria, desirably Gram-positive bacteria. In some aspects of this embodiment, the peptide or protein generated corresponds to an expression product encoded by a full-length gene, a portion of a full-length gene, or a random polynucleotide or fragment of DNA generated by oligonucleotide synthesis or enzymatic digestion of genomic DNA (e.g., bacteria, yeast, or mold).

In one embodiment of the present invention, a fusion promoter comprising an operator, such as xylO, and a promoter, such one selected from SEQ ID NOs.: 36-45, is operably linked to a nucleic acid to be transcribed from the promoter, the construct is introduced into bacteria, a solution of xylose or an analog thereof is provided (e.g., a 2.0% xylose solution), and induction of transcription proceeds. This expression method can be performed in Gram-positive or Gram-negative bacteria and are desirably performed in Staphylococcus, Enterococcus or other Gram-positive species.

The DNA encoding the desired molecule can be generated by oligonucleotide synthesis or enzymatic cleavage of DNA (e.g., random fragments of bacterial genomic DNA including, but not limited to, Staphylococcus) The peptides expressed by using a fusion promoter can be polypeptides, proteins, and fusion proteins. The peptides can be, for example, 3-20 amino acids in length, 20-100 amino acids in length, or 100, 200, 300, 400, 500, 600, 700, 800, or more amino acids in length. That is, the peptides can be at least 3, 4, 5, 6, 7, 8, 9, 10, 11, 12, 13, 14, 15, 16, 17, 18, 19, 20, 20, 21, 22, 23, 24, 25, 26, 27, 28, 29, 30, 31, 32, 33, 34, 35, 36, 37, 38, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 250, 300, 350, 400, 450, 500, 550, 600, 650, 700, 750, 800 or more than 800 amino acids in length.

Regulatable promoters providing a range of transcriptional levels in both the repressed and induced states, such as the promoters described herein, are desirable in applications where production of defined levels of transcript or protein is necessary. For example, promoters which can be used to tightly and/or finely regulate transcription at a desired level are desirable for use in cell-based assays which employ sensitized cells for screening potential antibiotic compounds (described in Examples 19-25). Specifically, it is desirable to use regulatable fusion promoters, such as those described herein, to modulate the sensitivity of cells to test compounds by altering the amount of a proliferation-inhibiting antisense nucleic acid transcribed by the promoter. Because the results of such cell-based assays are affected by the degree to which the cells are sensitized, the efficacy of these assays can be increased by using promoters having the ability to finely regulate the level of proliferation-inhibiting transcript over a wide range of transcript concentrations.

The present invention contemplates fusion promoters which can be used to achieve the desired level of nucleic acid transcription or protein expression desired for applications such as the cell-based gene and drug discovery methods described herein. The appropriate fusion promoter can be selected by first determining the approximate level of transcript production that is required for the assay. For example, if only a low level of transcription is necessary, a promoter with a low maximum transcription efficiency may be used. If a tightly controlable transcription over a wide range is required, a fusion promoter having nonleaky repression and a high maximum transcription efficiency may used. The amount of actual transcription obtained from the fusion promoter can then be fine tuned by determining the concentration of inducer which achieves the desired effect. Inducer can be introduced to the cell cultures as a solid or a liquid. The inducer can also be an appropriate growth condition, including but not limited to, the absense of a regulatory molecule, a change in temperature or exposure to radiation. The inducer can comprise from 0% to greater than 25% of the concentration of the growth medium or can be an appropriate growth condition. In some enbodiments, the inducer comprises 0%, 0.1%, 0.2%, 0.3%, 0.4%, 0.5%, 0.6%, 0.7%, 0.8%, 0.9%, 1.0%, 1.5%, 2.0%, 3.0%, 4.0%, 5.0%, 6.0%, 7.0%, 8.0%, 9.0%, 10.0%, 12.0%, 15%, 17%, 20%, 25%, or greater than 25% of the concentration of the growth medium.

The present invention includes vectors containing fusion promoters operably linked to a reporter gene. Such expression vectors can be used to quantitate the transcriptional activity of the fusion promoter contained therein. In some embodiments, a promoter can be inserted into the reporter vector as a cassette so as to form a fusion promoter construct with an operator located on the vector upstream of the reporter gene. In a prefered embodiment, the β-galactosidase encoding genes, lacL-lacM, from Leuconostoc mesenteroides, (Israelsen et al., Appl. Environ. Microbiol. 61:2540-2547 (1995); Pedersen et al., Mol. Gen. Genet. 244:374-382 (1994), the disclosures of which are incorporated by reference in their entireties), are linked to a fusion promoter described herein. The present invention also contemplates methods for using the lacL-lacM genes as a reporter in Enterococcus faecalis.

Expression vectors containing fusion promoters linked upstream of a multiple cloning site (MCS) and/or a transcription terminator are also contemplated by the present invention. In a preferred embodiment the transcription terminator is a rho-independent terminator such as rrnBt1t2. In some embodiments of the present invention, a promoter sequence can be inserted into the expression vector as a cassette so as to form a fusion promoter construct with an operator located on the vector upstream of the MCS. In other embodiments, a promoter linked to an operator can be inserted into the expression vector as a cassette just upstream of the MCS.

Expression vectors containing fusion promoters can be used to produce transcripts, such as antisense RNA, or for the expression of proteins. In some embodiments, nucleic acids corresponding to proliferation-required genes or fragments thereof or random genomic fragments are inserted into the MCS in a sense or antisense orientation. Upon induction of the fusion promoter with an inducer, antisense or sense transcripts are produced. Expression of such transcripts from the fusion promoter can be tightly and/or finely regulated.

In a preferred embodiment of the present invention, reporter and expression vectors contain both an origin of replication that is functional in Escherichia coli and/or other Gram-negative organisms and an origin of replication that function in one or more Gram-positive organisms such as Bacillus anthracis, Clostridium botulinum, Clostridium difficile, Clostridium perfringens, Clostridium teteni, Corynebacterium diptheriae, Enterococcus faecalis, Enterococcus faecium, Lactococcus lactis, Listeria monocytogenes, Mycobacterium leprae, Mycobacterium tuberculosis, Nocardia asteroides, Staphylococcus aureus, Staphylococcus epidermidis, Staphylococcus xylosis, Streptococcus pneumoniae, Streptococcus mutans or any species falling within the genera of any of the above species. In some embodiments, an origin of replication is functional in Staphylococcus aureus and Enterococcus faecalis.

In some embodiments, nucleic acids that are operably linked to the fusion promoter encode a molecule (e.g., a sense RNA, anti-sense RNA, aptamer, or peptide) that inhibits proliferation of Gram-positive bacteria including, but not limited to, Bacillus anthracis, Clostridium botulinum, Clostridium difficile, Clostridium perfringens, Clostridium teteni, Corynebacterium diptheriae, Enterococcus faecalis, Enterococcus faecium, Lactococcus lactis, Listeria monocytogenes, Mycobacterium leprae, Mycobacterium tuberculosis, Nocardia asteroides, Staphylococcus aureus, Staphylococcus epidermidis, Staphylococcus xylosis, Streptococcus pneumoniae, Streptococcus mutans or any species falling within the genera of any of the above species. Certain embodiments include methods of identifying genes that are essential to bacterial proliferation (“essential genes”) whereby the constructs described above are selectively induced in bacteria, such as pathogenic bacteria, at various levels and the impact on cell proliferation is monitored. For example, the fusion promoters of the present invention may be utilized in any of the methods described in U.S. patent application Ser. No. 09/492,709, U.S. patent application Ser. No. 09/711,164, U.S. patent application Ser. No. 09/741,699, and U.S. patent application Ser. No. 09/815,242, the disclosures of which are incorporated by reference herein in their entireties.

One such method is practiced by introducing a candidate nucleic acid into a microorganism, wherein the candidate nucleic acid is operably linked to a fusion promoter comprising a xyl, tet, trp, mal, lac or λc1 operator and one of the promoter sequences described herein. The fusion promoter may comprise a promoter that has been modified to to alter its transcriptional activity in one or more Gram-positive bacteria including but not limited to Bacillus anthracis, Clostridium botulinum, Clostridium difficile, Clostridium perfringens, Clostridium teteni, Corynebacterium diptheriae, Enterococcus faecalis, Enterococcus faecium, Lactococcus lactis, Listeria monocytogenes, Mycobacterium leprae, Mycobacterium tuberculosis, Nocardia asteroides, Staphylococcus aureus, Staphylococcus epidermidis, Staphylococcus xylosis, Streptococcus pneumoniae, Streptococcus mutans or any species falling within the genera of any of the above species. In some embodiments, the candidate nucleic acid is generated by random oligonucleotide synthesis and in other embodiments, the candidate nucleic acid is generated by cleavage of genomic DNA from an organism (e.g., bacteria, viruses, fungi, mold, parasites, yeast, plants and animals). For example, the candidate nucleic acid may be a random fragment generated by methods such as DNaseI digestion, restriction endonuclease digestion or shearing. In some cases, the candidate nucleic acid will be made so that an antisense RNA, sense RNA, aptamer, peptide, or protein will be expressed upon induction of the fusion promoter. In some cases, the candidate nucleic acid will encode a transcript that corresponds to or is complementary to a gene present in the genome of an organism (e.g., bacteria, viruses, fungi, mold, parasites, yeast, plants and animals). In other cases, the candidate nucleic acid will encode a peptide that corresponds to or interacts with a protein in the bacteria.

In the embodiments in which an aptamer is transcribed upon induction of the fusion promoter, the aptamer may be a nucleic acid which directly binds to the target molecules. Alternatively, the aptamer may encode a peptide that binds to the target molecule. For example, the aptamer may encode a peptide of at least 4, 6, 8, 10, 15, 20, 25, 30, 35, 40, 45, 50, 55, 60, 65, 70 or more than 70 amino acids in length. Aptamers which encode peptides have been described in PCT publication WO 99/50462, the disclosure of which is incorporated herein by reference in its entirety.

For example, RNA aptamers corresponding to regions of Tat and Rev RNA were introduced into or expressed in a cell and were shown to be specific RNA ligands for proteins that can specifically inhibit their translation. (Good et al., 1997, Gene Therapy 4: 45-54). Similarly, Conrad et al (J. Biol. Chem. 269: 32051, 1994) have shown that certain RNA aptamers can inhibit the βII isoenzyme of PKC. These RNA aptamers were selected from a pool of RNA molecules that contained a 120-nucleotide randomized region.

In general, the aspects of the invention that employ aptamer technology concern the use of the fusion promoters described herein to drive expression of aptamers of varying lengths and compositions. A minimum of approximately 6 nucleotides, preferably 10, and more preferably 14 or 15 nucleotides, may be necessary to effect specific binding to some target molecules. Thus, in some embodiments, fusion promoters of the invention are linked to oligonucleotides encoding aptamers that are 6, 10, 14, or 15 nucleotides in length. The only apparent limitations on the binding specificity of the target/aptamer complex concern sufficient sequence to be distinctive in the binding aptamer and sufficient binding capacity of the target molecule to obtain the necessary interaction. Aptamers of binding regions containing sequences shorter than 10, e.g., 6-mers, are feasible if the appropriate interaction can be obtained in the context of the environment in which the target is placed. Thus, if there are few interferences by other materials, less specificity and less strength of binding may be required.

Several methods for selecting aptamer oligonucleotides directed against a specific protein are known in the art and these approaches can be employed to design constructs having the fusion promoters linked to an oligonucleotide that encodes an aptamer. Examples of these methods include those methods described by Mills et al. Proc. Natl. Acad. Sci. USA 58:224 (1967), Green et al. Nature 347:406 (1990), and Tuerk and Gold Science 249:505 (1990), (all references are expressly incorporated by reference in their entireties), which show the use of in vitro evolution techniques to select RNA molecules with desired properties. Ellington and Szostak Nature 346:818 (1990), and Bock et al. Nature 355:564 (1992) (all references are expressly incorporated by reference in their entireties) also describe techniques useful for the selection of DNA oligonucleotides. More teachings on the design and selection of apatamers can be found in U.S. Pat. Nos. 5,756,291; 5,874,567; 5,874,281; 5,998,596; and 5,965,352 (all references are expressly incorporated by reference in their entireties). The field has also been recently reviewed by Burke and Berzal-Herranz FASEB J 7:106 (1993), herein expressly incorporated by reference in its entirety.

Once the construct comprising the candidate nucleic acid operably linked to a promoter of the invention is made, it is transferred to host bacteria. Next, the proliferation of microorganisms transcribing a higher level of the candidate nucleic acid is compared to the proliferation of microorganisms that transcribe a lower level of the candidate nucleic acid or that do not transcribe the candidate nucleic acid. These varying levels of transcription can be achieved by varying the amounts of inducer (e.g., xylose, tetracycline, maltose, the absense of tryptophan, temperature, exposure to ultraviolet radiation, or analogs thereof). The level of maximal induction that can be achieved will depend on the fusion promoter selected. A difference in cell proliferation in the sample transcribing a higher level of the candidate nucleic acid compared to the sample expressing a lower level of the candidate nucleic acid indicates that the candidate nucleic acid encodes a nucleic acid that is complementary to an essential gene or encodes a peptide or protein that reduces proliferation of the microorganism.

In some embodiments, the RNA, aptamer, peptide or protein reduces proliferation of the microorganisms by interacting with another molecule required for proliferation. In this case, many techniques in molecular biology can be employed to identify the cell proliferation molecule that interacts with the aptamer, peptide, or protein and the gene encoding this molecule. Conventional one and two hybrid systems, for example, can be readily adapted to identify molecules that bind to an aptamer, peptide, or protein described above, for example. Such approaches include:

(1) the two-hybrid systems (Field & Song, Nature 340:245-246 (1989); Chien et al., Proc. Natl. Acad. Sci. USA 88:9578-9582 (1991); and Young K H, Biol. Reprod. 58:302-311 (1998), all of which are expressly incorporated by reference in their entirety);

(2) the reverse two-hybrid system (Leanna & Hannink, Nucl. Acid Res. 24:3341-3347 (1996), herein incorporated by reference in their entirety);

(3) the repressed transactivator system (Sadowski et al., U.S. Pat. No. 5,885,779), herein incorporated by reference in their entirety);

(4) phage display systems (Lowman H B, Annu. Rev. Biophys. Biomol. Struct. 26:401-424 (1997), herein incorporated by reference in their entirety); and

(5) GST/HIS pull down assays, mutant operators (Granger et al., WO 98/01879) and the like (See also Mathis G., Clin. Chem. 41:139-147 (1995); Lam K. S. Anticancer Drug Res., 12:145-167 (1997); and Phizicky et al., Microbiol Rev. 59:94-123 (1995), all of which are expressly incorporated by reference in their entirety).

In one embodiment, the present invention utilizes an antisense-based method to identify proliferation-required sequences. Generally, a library of nucleic acids from a given source are subcloned or otherwise inserted immediately downstream of an inducible fusion promoter on an appropriate vector, such as one of the expression vectors described herein, thus forming an expression library. It is generally preferred that expression is directed by a regulatable fusion promoter such that expression level can be adjusted by addition of variable concentrations of an inducer molecule or of an inhibitor molecule to the medium. Temperature activated fusion promoters, such as promoters regulated by temperature sensitive repressors, such as the lambda C₁₈₅₇ repressor, are also envisioned. Although the insert nucleic acids may be derived from the chromosome of the cell or microorganism into which the expression vector is to be introduced, because the insert is not in its natural chromosomal location, the insert nucleic acid is an exogenous nucleic acid for the purposes of the discussion herein.

Once generated, the expression library containing the nucleic acids is introduced into a population of cells (such as the organism from which the nucleic acid sequences were obtained or an organism other than the organism from which the sequences were obtained) to search for genes that are required for bacterial proliferation. Because the library molecules are foreign, in context, to the population of cells, the expression vectors and the nucleic acid segments contained therein are considered exogenous nucleic acid.

Expression of the nucleic acid fragments in the test population of cells containing the expression library is then activated. Activation of the expression vectors consists of subjecting the cells containing the vectors to conditions that result in the expression of the nucleic acids carried by the expression library. The test population of cells is then assayed to determine the effect of expressing the nucleic acid fragments on the test population of cells. Those expression vectors that negatively impact the growth of the cells upon induction of expression of the random sequences contained therein are identified, isolated, and purified for further study.

A variety of assays are contemplated to identify nucleic acid sequences that negatively impact growth upon expression. In one embodiment, growth in cultures expressing nucleic acid sequences and growth in cultures not expressing these sequences is compared. Growth measurements are assayed by examining the extent of growth by measuring optical densities. Alternatively, enzymatic assays can be used to measure bacterial growth rates to identify nucleic acid sequences of interest. Colony size, colony morphology, and cell morphology are additional factors used to evaluate growth of the host cells. Those cultures that fail to grow or grow at a reduced rate under expression conditions are identified as containing an expression vector encoding a nucleic acid fragment that negatively affects a proliferation-required gene.

In some embodiments, genomic fragments of an organism are operably linked to an inducible fusion promoter in a vector and assayed for growth inhibition activity. Examples below describe the examination of libraries of genomic fragments cloned into vectors comprising inducible fusion promoters. Vectors containing fusion promoters, such as those described herein, can be used. Upon transformation of these vectors into a host organism and induction, the vectors produce an RNA molecule corresponding to the subcloned genomic fragments. In those instances where a genomic fragment is in an antisense orientation with respect to the fusion promoter, the antisense transcript produced can be complementary to at least a portion of a sense messenger RNA (mRNA) encoding a proliferation-required gene product such that the antisense transcript interacts with at least a portion of the proliferation-required sense mRNA produced by the host organism thereby decreasing the translation efficiency or the level of the proliferation-required sense mRNA and thus decreasing production of the proliferation-required protein encoded by the sense mRNA molecule. In cases where the sense mRNA encodes a protein required for proliferation, the host organism containing a vector from which transcription from the fusion promoter has been induced fails to grow or grows at a substantially reduced rate. Additionally, in cases where the transcript produced is complementary to at least a portion of a non-translated RNA and where that non-translated RNA is required for proliferation, host cells containing a vector from which transcription from the fusion promoter has been induced also fail to grow or grow at a substantially reduced rate.

In cases where the candidate nucleic acid encodes an antisense nucleic acid comprising a nucleotide sequence that is complementary to a gene required for proliferation or a portion thereof, the gene sequence of the coding fragment of the proliferation-required gene or a portion thereof can be determined by direct sequencing.

Determination of the gene(s) corresponding to the nucleotide sequence can achieved by comparing the obtained sequence data with databases containing known protein and nucleotide sequences from various microorganisms. Thus, initial gene identification can made on the basis of significant sequence similarity or identity to either characterized or predicted from Staphylococcus aureus and Enterococcus faecalis genes or their encoded proteins and/or homologues in other species.

The number of nucleotide and protein sequences available in database systems has been growing exponentially for years. For example, the complete nucleotide sequences of Caenorhabditis elegans and several bacterial genomes, including E. coli, Aeropyrum pernix, Aquifex aeolicus, Archaeoglobus fulgidus, Bacillus subtilis, Borrelia burgdorferi, Chlamydia pneumoniae, Chlamydia trachomatis, Clostridium tetani, Corynebacterium diptheria, Deinococcus radiodurans, Haemophilus influenzae, Helicobacter pylori 26695, Helicobacter pylori J99, Methanobacterium thermoautotrophicum, Methanococcus jannaschii, Mycobacterium tuberculosis, Mycoplasma genitalium, Mycoplasma pneumoniae, Pseudomonas aeruginosa, Pyrococcus abyssi, Pyrococcus horikoshii, Rickettsia prowazekii, Synechocystis PCC6803, Thermotoga maritima, Treponema pallidum, Bordetella pertussis, Campylobacter jejuni, Clostridium acetobutylicum, Mycobacterium tuberculosis CSU#93, Neisseria gonorrhoeae, Neisseria meningitidis, Pseudomonas aeruginosa, Pyrobaculum aerophilum, Pyrococcus furiosus, Rhodobacter capsulatus, Salmonella typhimurium, Streptococcus mutans, Streptococcus pyogenes, Ureaplasma urealyticum and Vibrio cholera are available. This nucleotide sequence information is stored in a number of databanks, such as GenBank, the National Center for Biotechnology Information (NCBI), the Genome Sequencing Center (http://genome.wustl.edu/gsc/salmonella.shtml), and the Sanger Centre (http://www.sanger.ac.uk/projects/S_typhi) which are publicly available for searching.

A variety of computer programs are available to assist in the analysis of the sequences stored within these databases. FASTA, (W. R. Pearson (1990) “Rapid and Sensitive Sequence Comparison with FASTP and FASTA” Methods in Enzymology 183:63-98), Sequence Retrieval System (SRS), (Etzold & Argos, SRS an indexing and retrieval tool for flat file data libraries. Comput. Appl. Biosci. 9:49-57, 1993) are two examples of computer programs that can be used to analyze sequences of interest. In one embodiment of the present invention, the BLAST family of computer programs, which includes BLASTN version 2.0 with the default parameters, or BLASTX version 2.0 with the default parameters, is used to analyze nucleotide sequences.

BLAST, an acronym for “Basic Local Alignment Search Tool,” is a family of programs for database similarity searching. The BLAST family of programs includes: BLASTN, a nucleotide sequence database searching program, BLASTX, a protein database searching program where the input is a nucleic acid sequence; and BLASTP, a protein database searching program. BLAST programs embody a fast algorithm for sequence matching, rigorous statistical methods for judging the significance of matches, and various options for tailoring the program for special situations. Assistance in using the program can be obtained by e-mail at blast@ncbi.nlm.nih.gov. tBLASTX can be used to translate a nucleotide sequence in all three potential reading frames into an amino acid sequence.

Bacterial genes are often transcribed in polycistronic groups. These groups comprise operons, which are a collection of genes and intergenic sequences under common regulation. The genes of an operon are transcribed on the same mRNA and are often related functionally. Given the nature of the screening protocol, it is possible that the identified exogenous nucleic acid corresponds to a gene or portion thereof with or without adjacent noncoding sequences, an intragenic sequence (i.e. a sequence within a gene), an intergenic sequence (i.e. a sequence between genes), a nucleotide sequence spanning at least a portion of two or more genes, a 5′ noncoding region or a 3′ noncoding region located upstream or downstream from the actual nucleotide sequence that is required for bacterial proliferation. Accordingly, it is often desirable to determine which gene(s) that is encoded within the operon is individually required for proliferation.

In one embodiment of the present invention, an operon is identified and then dissected to determine which gene or genes are required for proliferation. Operons can be identified by a variety of means known to those in the art. For example, the RegulonDB DataBase described by Huerta et al. (Nucl. Acids Res. 26:55-59, 1998), which may also be found on the website http://www.cifn.unam.mx/Computational_Biology/regulondb/, the disclosures of which are incorporated herein by reference in their entireties, provides information about operons in Escherichia coli. The Subtilist database (http://bioweb.pasteur.fr/GenoList/SubtiList), (Moszer, I., Glaser, P. and Danchin, A. (1995) Microbiology 141: 261-268 and Moszer, 1 (1998) FEBS Letters 430: 28-36, the disclosures of which are incorporated herein in their entireties), may also be used to predict operons. This database lists genes from the fully sequenced, Gram-positive bacteria, Bacillus subtilis, together with predicted promoters and terminator sites. This information can be used in conjunction with the Staphylococcus aureus genomic sequence data to predict operons and thus produce a list of the genes affected by the antisense nucleic acids of the present invention. The TIGR microbial database has an incomplete version of the Enterococcus faecalis genome http://www.tigr.org/cgi-bin/BlastSearch/blast.cgi?organism=e_faecalis. One can take a nucleotide sequence and BLAST it for homologs.

A number of techniques that are well known in the art can be used to dissect the operon. Analysis of RNA transcripts by Northern blot or primer extension techniques are commonly used to analyze operon transcripts. In one aspect of this embodiment, gene disruption by homologous recombination is used to individually inactivate the genes of an operon that is thought to contain a gene required for proliferation.

Several gene disruption techniques have been described for the replacement of a functional gene with a mutated, non-functional (null) allele. These techniques generally involve the use of homologous recombination. One technique using homologous recombination in Staphylococcus aureus is described in Xia et a. 1999, Plasmid 42: 144-149, the disclosure of which is incorporated herein by reference in its entirety. This technique uses crossover PCR to create a null allele with an in-frame deletion of the coding region of a target gene. The null allele is constructed in such a way that nucleotide sequences adjacent to the wild type gene are retained. These homologous sequences surrounding the deletion null allele provide targets for homologous recombination so that the wild type gene on the Staphylococcus aureus chromosome can be replaced by the constructed null allele. Enterococcus faecalis genes can be disrupted by recombining in a non-replicating plasmid that contains an internal fragment to that gene (Leboeuf, C., L. Leblanc, Y. Auffray and A. Hartke. 2000. J. Bacteriol. 182:5799-5806, the disclosure of which is incorporated herein by reference in its entirety).

The crossover PCR amplification product is subcloned into a suitable vector having a selectable marker, such as a drug resistance marker. In some embodiments the vector may have an origin of replication which is functional in Escherichia coli or another organism distinct from the organism in which homologous recombination is to occur, allowing the plasmid to be grown in Escherichia coli or the organism other than that in which homologous recombination is to occur, but may lack an origin of replication functional in Staphylococcus aureus or Enterococcus faecalis such that selection of the selectable marker requires integration of the vector into the homologous region of the Staphylococcus aureus or Enterococcus faecalis chromosome. Usually a single crossover event is responsible for this integration event such that the Staphylococcus aureus or Enterococcus faecalis chromosome now contains a tandem duplication of the target gene consisting of one wild type allele and one deletion null allele separated by vector sequence. Subsequent resolution of the duplication results in both removal of the vector sequence and either restoration of the wild type gene or replacement by the in-frame deletion. The latter outcome will not occur if the gene should prove essential. A more detailed description of this method is provided below. It will be appreciated that this method may be practiced with any genes identified as required for proliferation in any of the organisms described herein.

Bacteria that harbor the expression vectors described herein can be induced to transcribe a desired level of RNA or to express a desired level of peptide, which affects a gene that is essential for the viability and/or growth of the cell and renders the cells more sensitive to compounds that reduce cellular proliferation. This technique is referred to as Essential Gene Interference (EGI). In one embodiment, for example, an RNA, such as an antisense RNA, or peptide generated from the constructs described above interacts with an essential gene of a bacteria, RNA, or product thereof and when the level of transcript or peptide is increased in the cell, viability and/or cell growth is compromised. Thus, the genes that are affected by the transcript or peptide either directly or indirectly (e.g., by interacting with the RNA or protein that correspond to the gene) causing a reduction in viability and/or cell growth are identified as essential genes. These essential genes are then used as drug discovery targets for high throughput drug screening.

The promoters described herein can also be used to replace endogenous promoters so as to create cells having specific regulatable genes. By this approach, an essential gene is identified (e.g., using the techniques described herein) and the endogenous promoter is replaced with a regulatable promoter of the invention (e.g, Xyl-T5, Xyl-T5-DD, Xyl-CP25, Xyl-P32, Xyl-P59, Xyl-P59*, Xyl-P1P2, Xyl-P1P2*, Xyl, PL, and XylPL10) using ET cloning technology. This technique is referred to as Rapid Replacement of Genomic Promoters (promoter replacement). This method employs the Polymerase Chain Reaction (PCR) to replace the promoter of the target gene without interrupting the endogenous Shine-Dalgamo sequence. As little as 60 bp homology to the target promoter can be used. The correct integration of the cassette is confirmed by colony PCR and correct integrants are tested for growth defects when an inducer is titrated away. The inability to grow in the absence of inducer confirms the essentiality of the target gene. Additionally, the bacterial strains created using this approach can be used in high throughput drug screening methodologies.

A similar technique to promoter replacement can be used to insert an operator into a genomic promoter region so as to make the genomic promoter regulatable. This technique is referred to as operator insertion. As little as 60 bp homology to the target promoter region can be used. The correct integration of the cassette is confirmed by colony PCR and correct integrants are tested for growth defects when an inducer is titrated away. The inability to grow in the absence of inducer confirms the essentiality of the target gene. Additionally, the bacterial strains created using this approach can be used in high throughput drug screening methodologies.

In some embodiments, promoter replacement and operator insertion methods are conducted in cells which have an increased frequency of homologous recombination. For example, the organism may lack or have a reduced level or activity of one or more exonucleases which would ordinarily degrade the DNA to be inserted into the chromosome, thereby effectively increasing the stability of the linear DNA in the organism.

In still further embodiments, the strains may both lack or have reduced levels of exonucleases and constitutively or conditionally express proteins involved in mediating homologous recombination. For example, Gram-positive bacterial strains in which the activity of an exonuclease which degrades linear nucleic acids, such as exonuclease V of the RecBCD recombination pathway or an exonuclease with an analogous function, has been reduced or eliminated, may be used. Such strains include those having mutations in the recB, recC, or recD genes or genes that express proteins having analogous functions, that enhance the frequency of homologous recombination. In some embodiments, the strains have mutations in more than one of the recB, recC, or recD genes or genes that express proteins having analogous functions, that enhance the frequency of homologous recombination. For example the strains may have mutations in both the recB and recC genes or genes that express proteins having analogous functions.

It will be appreciated that Gram-positive organisms having reduced or eliminated levels of one or more proteins analogous in function to RecB, RecC, or RecD may be used with the methods described herein. In some embodiments, Gram-positive organisms having reduced or eliminated levels of one or more proteins analogous in function to RecB, RecC or RecD may be selected from the group consisting of Bacillus anthracis, Clostridium botulinum, Clostridium difficile, Clostridium perfringens, Clostridium teteni, Corynebacterium diptheriae, Enterococcus faecalis, Enterococcus faecium, Lactococcus lactis, Listeria monocytogenes, Mycobacterium leprae, Mycobacterium tuberculosis, Nocardia asteroides, Staphylococcus aureus, Staphylococcus epidermidis, Staphylococcus xylosis, Streptococcus pneumoniae, Streptococcus mutans or any species falling within the genera of any of the above species. In some embodiments, it may be desirable to use Escherichia coli having reduced or eliminated levels of one or more of RecB, RecC or RecD proteins as a surrogate host in the methods described herein.

The methods may also be performed in Gram-positive bacterial strains in which the activity of a the RecET recombinase system of the Rac prophage, or a recombinase system having an analogous function, has been activated. Such strains are analogous to Escherichia coli strains which carry an sbcA mutation. The RecE gene of the Rac prophage encodes ExoVIII a 5′-3′ exonuclease, while the RecT gene of the Rac prophage encodes a single stranded DNA binding protein which facilitates renaturation and D-loop formation. Thus, the gene products of the RecE and RecT genes or proteins with analogous functions facilitate homologous recombination. The RecE and RecT genes are on the same operon and are not normally expressed. Similarly, genes having a function analogous to the RecE and RecT may not be expressed. However, strains having a mutation analogous to sbcA would activate the expression of RecE and RecT or genes having analogous function. In some embodiments, the methods may be performed in strains which carry mutations in the recB and recC genes or genes that express proteins having analogous functions, as well as the mutation in the gene analogous in function to sbcA.

In some embodiments, the methods may be performed in Gram-positive bacterial strains in which recombination via the RecF pathway or a pathway having an analogous function has been enhanced. One example of such strains are those having a mutation analogous in function to the Escherichia coli sbcB mutation.

It will be appreciated that the recE and recT gene products, or proteins with analagous functions may be constitutively or conditionally expressed in Gram-positive organisms. In some embodiments, these proteins may be conditionally or constitutively expressed in Gram-positive organisms such as Bacillus anthracis, Clostridium botulinum, Clostridium difficile, Clostridium perfringens, Clostridiurn teteni, Corynebacterium diptheriae, Enterococcus faecalis, Enterococcus faecium, Lactococcus lactis, Listeria monocytogenes, Mycobacterium leprae, Mycobacterium tuberculosis, Nocardia asteroides, Staphylococcus aureus, Staphylococcus epidermidis, Staphylococcus xylosis, Streptococcus pneumoniae, Streptococcus mutans or any species falling within the genera of any of the above species. In some embodiments, Gram-positive strains may be genetically engineered to express the RecE and recT genes of the Rac prophage. For example, plasmids encoding these gene products may be introduced into the organism. If desired, the coding sequences encoding these gene products may be optimized to reflect the codon preferences of the organism in which they are to be expressed. In some embodiments, it may be desirable to use Escherichia coli constitutively or conditionally expressing the RecE and RecT gene products as a surrogate host in the methods described herein.

In further embodiments, the methods may be conducted in cells which utilize the Red system of bacteriophage lambda (λ) or analogous systems from other phages to enhance the frequency of homologous recombination. The Red system contains three genes, γ, β and exo whose products are the Gam, Bet and Exo proteins (see Ellis et al. PNAS 98:6742-6746, 2001, the disclosure of which is incorporated herein by reference in its entirety). The Gam protein inhibits the RecBCD exonuclease V, thus permitting Bet and Exo to gain access to the ends of the DNA to be integrated and facilitating homologous recombination. The Bet protein is a single stranded DNA binding protein. The Exo protein is a double-stranded DNA dependent 5′-3′ exonuclease. Thus, expression of the λ red proteins or proteins having analogous functions facilitates homologous recombination.

It will be appreciated that the λ Bet, Gam and Exo proteins, or proteins with analagous functions may be constitutively or conditionally expressed in Gram-positive organisms. In some embodiments, these proteins may be conditionally or constitutively expressed in Gram-positive organisms such as Bacillus anthracis, Clostridium botulinum, Clostridium difficile, Clostridium perfringens, Clostridium teteni, Corynebacterium diptheriae, Enterococcus faecalis, Enterococcus faecium, Lactococcus lactis, Listeria monocytogenes, Mycobacterium leprae, Mycobacterium tuberculosis, Nocardia asteroides, Staphylococcus aureus, Staphylococcus epidermidis, Staphylococcus xylosis, Streptococcus pneumoniae, Streptococcus mutans or any species falling within the genera of any of the above species. For example, plasmids encoding these gene products may be introduced into the organism. If desired, the coding sequences encoding these gene products may be optimized to reflect the codon preferences of the organism in which they are to be expressed. In some embodiments, it may be desirable to use Escherichia coli constitutively or conditionally expressing the λ Bet, Gam and Exo gene products as a surrogate host in the methods described herein.

In some embodiments, the cells may have an increased frequency of homologous recombination as a result of more than one of the aforementioned characteristics. In some embodiments, the enhanced frequency of recombination may be a conditional characteristic of the cells which depends on the culture conditions in which the cells are grown. For example, in some embodiments, expression of the λ Red Gam, Exo, and Bet proteins or recE and recT proteins may be constitutive or regulated. Thus, the cells may have an increased frequency of homologous recombination as a result of any combination of the aforementioned characteristics. For example, in some embodiments, the cell may carry the sbcA and recBC mutations or analogous mutations.

In some embodiments, a linear double stranded DNA containing the regulatable fusion promoter, operator or other regulatory element to be inserted into the chromosome of the prokaryotic organism is introduced into an organism constitutively or conditionally expressing the recE and recT or the λ Bet, Gam and Exo proteins or proteins with analogous functions as described above. In some embodiments, the double stranded DNA may be introduced into an organism having the recBC and sbcA mutations or analogous mutations.

In other embodiments, a single stranded DNA containing the regulatable fusion promoter, operator or other regulatory element to be inserted into the chromosome of the prokaryotic organism is introduced into an organism constitutively or conditionally expressing the λ Bet protein or a protein with an analogous function. In some embodiments the single stranded DNA is introduced into an organism constitutively or conditionally expressing both the k Bet and Gam proteins or proteins with analogous functions. In further embodiments, the single stranded DNA is introduced into an organism constitutively or conditionally expressing the λ Bet, Gam and Exo proteins or proteins with analogous functions.

It will be appreciated that any method of enhancing the frequency of homologous recombination may be used in conjunction with the present invention and that the methods of the present invention are not limited to use in cells having the aforementioned characteristics.

The 5′ and 3′ regions of the linear nucleic acid to be integrated via homologous recombination may have any length of homology to the target sequence in the chromosome of the organism which permits homologous recombination to occur. In some embodiments, the nucleic acid to be integrated via homologous recombination is generated by PCR. In such embodiments, it is desirable for the PCR amplicon to be relatively short. Accordingly, in some embodiments, the 5′ and 3′ regions of the amplicon which have homology to the target sequence in the chromosome of the organism, may each be more than 120 nucleotides in length, at least 120 nucleotides in length, at least 100 nucleotides in length, at least 90 nucleotides in length, at least 80 nucleotides in length, at least 70 nucleotides in length, at least 60 nucleotides in length, at least 50 nucleotides in length, at least 40 nucleotides in length, at least 30 nucleotides in length or less than 30 nucleotides in length.

As discussed above, the linear double stranded DNA or linear single stranded DNA may be introduced into a suitable strain having an enhanced frequency of recombination.

In one embodiment of the present invention, genes required for proliferation are identified by methods in which the native promoter of a gene is replaced with a regulatable fusion promoter using homologous recombination. In such methods, a linear promoter replacement construct which comprises a 5′ portion homologous to a first portion of the native promoter, a 3′ portion homologous to a second portion of the native promoter and a regulatable fusion promoter disposed between the 5′ portion and the 3′ portion is introduced into a cell containing the target gene. After replacement of the native promoter in the target gene with the regulatable fusion promoter via homologous recombination, the extent of proliferation of cells in which the regulatable fusion promoter has replaced the native promoter under conditions in which the regulatable fusion promoter is active at a first level is compared to the extent of proliferation of the cells under conditions in which the regulatable fusion promoter is active at a second level which is lower than the first level. If the candidate gene is required for proliferation, the extent of proliferation of the cells grown under conditions where the fusion promoter is active at the higher level will be greater than the extent of proliferation of cells grown under conditions where the fusion promoter is active at the lower level.

The regulatable fusion promoter in the promoter replacement construct may be any of the regulatable fusion promoters described herein. For example, in some embodiments, the regulatable fusion promoter may be selected from the group consisting of Xyl-T5, Xyl-T5-DD, Xyl-CP25, Xyl-P32, Xyl-P59, Xyl-P59*, Xyl-P1P2, Xyl-P1P2*, Xyl-PL, and Xyl-PL10.

In some embodiments, the linear promoter replacement construct may further comprise a fusion promoter operably linked to a nucleic acid which encodes an identifiable marker or a selectable marker. The promoter which is operably linked to a nucleic acid encoding an identifiable marker or a selectable marker and the nucleic acid which encodes the identifiable marker or selectable marker may be 5′ of the regulatable fusion promoter. The identifiable marker may be any protein whose expression is readily detectable. For example, the identifiable marker may be β-galactosidase, or any of the markers familiar to those skilled in the art which allow identification of cells expressing the marker. Likewise, the selectable marker may be any protein which allows selection of cells which express the protein. For example, the selectable marker may confer resistance to an antibiotic or permit growth under a particular set of culture conditions. A variety of selectable markers are familiar to those skilled in the art, including genes which confer resistance to antibiotics such as chloramphenicol, ampicillin, kanamycin and tetracycline.

In some embodiments, a transcriptional terminator, such as the rrnB terminator may be positioned between the 3′ end of the nucleic acid encoding the identifiable marker or selectable marker and the 5′ end of the regulatable fusion promoter.

The promoter replacement method may be performed in any desired organism. For example, in some embodiments, the method may be practiced in Gram-positive bacteria. In certain embodiments, the cell to be used with any of the methods described herein is selected from the group consisting of Bacillus anthracis, Clostridium botulinum, Clostridium difficile, Clostridium perfringens, Clostridium teteni, Corynebacterium diptheriae, Enterococcus faecalis, Enterococcus faecium, Lactococcus lactis, Listeria monocytogenes, Mycobacterium leprae, Mycobacterium tuberculosis, Nocardia asteroides, Staphylococcus aureus, Staphylococcus epidermidis, Staphylococcus xylosis, Streptococcus pneumoniae, Streptococcus mutans or any species falling within the genera of any of the above species.

In some embodiments, the promoter replacement method is conducted in cells which have an increased frequency of homologous recombination. For example, in some embodiments, the method is conducted in cells which carry the sbcA and recBC mutations, or in cells having mutations which confer similar phenotypes. In other embodiments, the cells may express one or more proteins in the λ Red recombination system. For example, in some embodiments the cells may constitutively or conditionally express the λ Gam, Bet, and Exo proteins or other proteins having similar functions. In other embodiments, the cells may constitutively or conditionally express the λ Bet protein or a protein having a similar function.

In some embodiments, a double stranded promoter replacement construct is introduced into a cell comprising the recBCD and sbcA mutations, recBC and sbcA mutations or mutations with analogous consequences. The double stranded promoter replacement construct may or may not comprise a gene encoding a selectable or identifiable marker, but in preferred embodiments the double stranded promoter replacement construct includes a gene enocding a selectable or identifiable marker. In other embodiments, a double stranded promoter replacement construct is introduced into a cell constitutively or conditionally expressing the recE and recT proteins or proteins with analogous functions. In still other embodiments, a double stranded promoter replacement construct is introduced into a cell constitutively or conditionally expressing the λ Gam, Bet, and Exo proteins or other proteins having similar functions. In such embodiments, cells in which the promoter replacement construct has integrated are identified by obtaining colonies which express the identifiable marker or by growing the cells in appropriate selective media. If desired, homologous integration may be confirmed by performing a Southern blot or amplification reaction using a probe or primers which will yield a known result if homologous recombination has occurred.

In other embodiments, a promoter replacement construct may be introduced into the cell as a single stranded DNA. Preferably, the single stranded promoter replacement construct does not comprise a gene which encodes an identifiable or selectable marker, but in some embodiments a gene which encodes an identifiable or selectable marker may be included in the construct. In some embodiments, the single stranded nucleic acid may be generated by in vitro synthesis. In other embodiments, a circular single stranded DNA, such as M13, comprising the promoter replacement construct may be fragmented by sonication and the fragments may be introduced into the cell. In still other embodiments, a single stranded promoter replacement construct is introduced into a cell constitutively or conditionally expressing the λ Bet protein, the Bet and Gam proteins, the Bet, Gam and Exo proteins or other proteins having similar functions. Cells in which integration has occurred at the desired location are identified by performing colony hybridization, amplification reactions, or other methods familiar to those skilled in the art.

As discussed above, the 5′ and 3′ portions of the linear promoter replacement construct which have homology to the promoter region of the target gene may have any length which permits homologous recombination to occur. Accordingly, in some embodiments, the 5′ and 3′ regions of the amplicon which have homology to the promoter region of the target gene in the chromosome of the organism, may each be more than 120 nucleotides in length, at least 120 nucleotides in length, at least 100 nucleotides in length, at least 90 nucleotides in length, at least 80 nucleotides in length, at least 70 nucleotides in length, at least 60 nucleotides in length, at least 50 nucleotides in length, at least 40 nucleotides in length, at least 30 nucleotides in length or less than 30 nucleotides in length.

In some embodiments, the promoter replacement construct is introduced into the cell as a linear nucleic acid. In particular, in some embodiments, the promoter replacement construct is generated by an conducting amplification reaction, such as a PCR reaction, to generate a linear amplicon. In some aspects of this embodiment, the PCR reaction is performed on a linearized plasmid. In a preferred aspect of this embodiment, the amplicon contains about 60 bp of homology to the target gene at both the 5′ and 3′ ends of the construct.

Thus, one embodiment of the invention relates to a method for identifying a gene which is required for proliferation of a cell comprising replacing the native promoter of a gene with a regulatable fusion promoter and identifying cells in which the extent of proliferation of the cell when the fusion promoter is active at a first level is substantially different than the extent of proliferation of the cell when the fusion promoter is active at a second level, the first level being greater than the second level. In some embodiments, the cell has an enhanced frequency of homologous recombination. In some embodiments, the fusion promoter is an inducible promoter. In other embodiments, the level of transcription from the promoter varies with the level of inducer present in the medium in which the cell is grown. In certain embodiments, the step of replacing the native promoter comprises introducing a linear nucleic acid comprising a 5′ portion homologous to a first portion of the native promoter, a 3′ portion homologous to a second portion of the native promoter and the regulatable promoter disposed between the 5′ portion and the 3′ portion into a cell such that homologous recombination occurs between the 5′ portion and the first portion of the native promoter and between the 3′ portion and the second portion of the native promoter. The nucleic acid may, in some embodiments, further comprise a gene encoding a selectable marker. The regulatable fusion promoter may be any of the fusion promoters described herein. In some embodiments, the 5′ portion comprises at least 60 nucleotides homologous to the first portion of the native promoter and the 3′ portion comprises at least 60 nucleotides homologous to the second portion of the native promoter. Certain embodiments include those in which the nucleic acid is linear. In some embodiments, the step of identifying cells comprises identifying cells which proliferate to a greater extent in the presence of inducer than in the absence of inducer. In other embodiments, the Shine Delgamo sequence of said native promoter is not replaced.

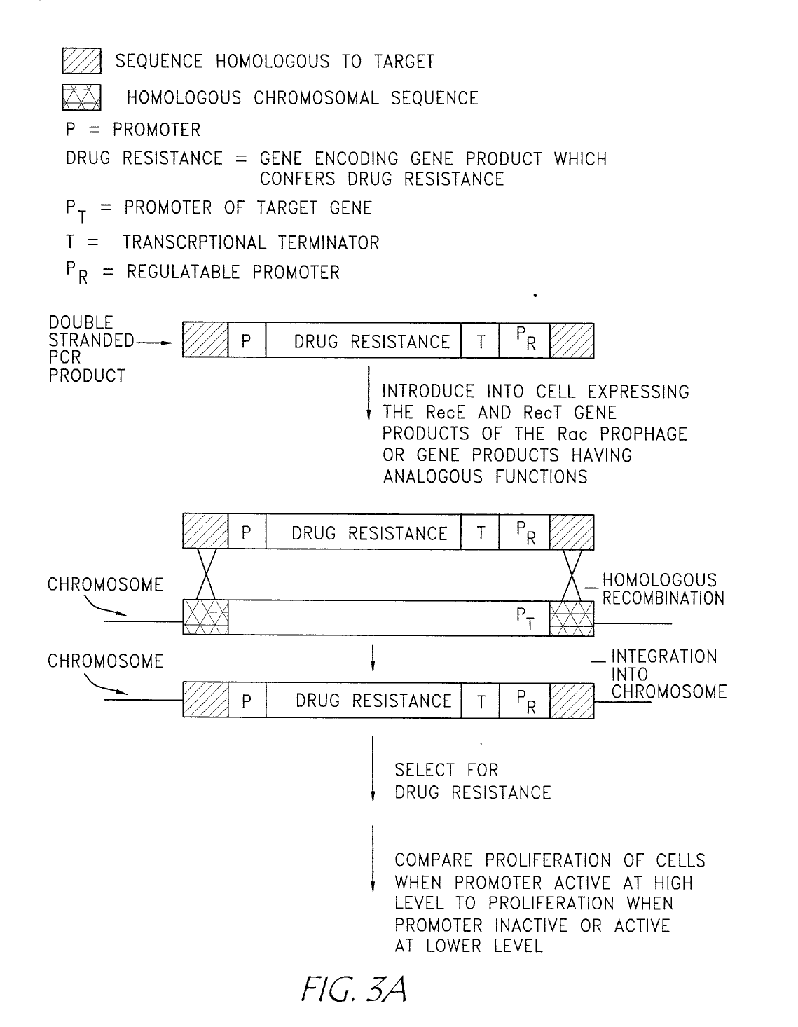

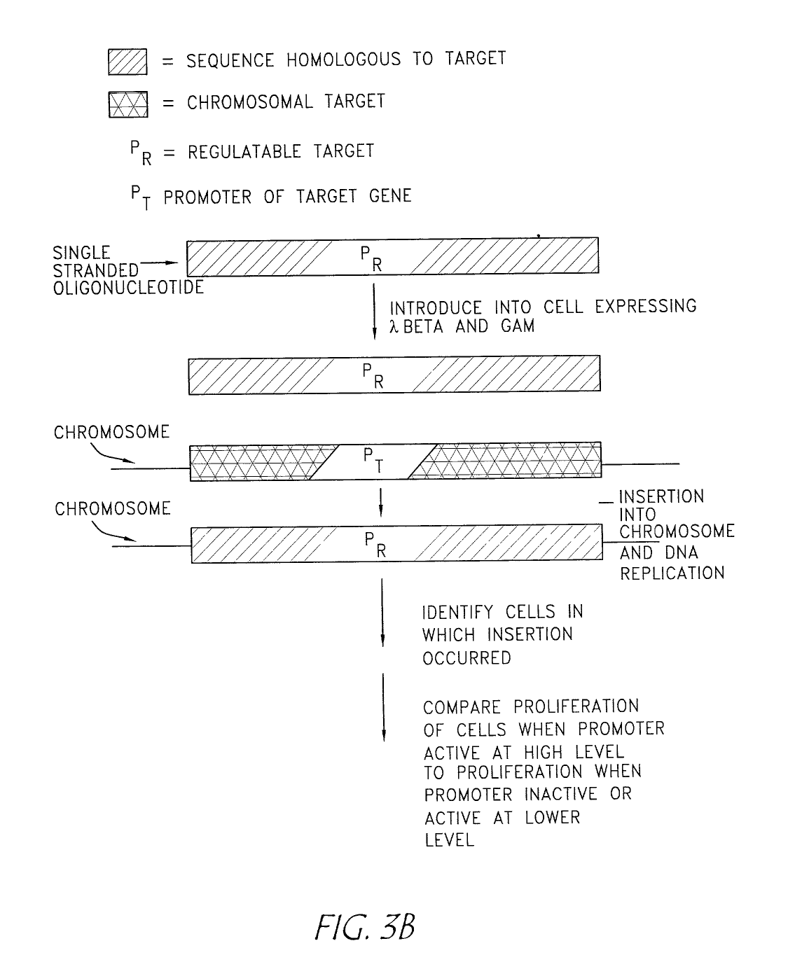

FIG. 3A illustrates one embodiment of the present promoter replacement method in which a linear double stranded promoter replacement construct comprising a nucleic acid encoding a selectable marker, a transcriptional terminator, and a regulatable fusion promoter is inserted into the chromosome of a cell expressing the RecE and RecT gene products of the Rac prophage or gene products having analogous functions. FIG. 3B illustrates one embodiment of the present promoter replacement method in which a linear single stranded promoter replacement construct comprising a 5′ portion homologous to a target sequence, a 3′ portion homologous to a target sequence and a regulatable fusion promoter is inserted into the chromosome of a cell expressing the λ Beta and Gam proteins.

As discussed below, the promoter replacement method may also be used to obtain cells in which the level or activity of a gene product required for proliferation is reduced. Such cells may be used in cell-based assays to identify compounds which inhibit proliferation.

In another embodiment of the present invention, genes required for proliferation are identified by methods in which a an operator is integrated into the promoter of a target gene or into the target gene itself via homologous recombination. The operator may be any nucleic acid which allows the activity or level of the gene product encoded by the target gene to be modulated. For example, in some embodiments, the operator may be recognized by a repressor. In such methods, a linear operator insertion construct which comprises a 5′ portion homologous to a first portion of the target promoter or gene, a 3′ portion homologous to a second portion of the target promoter or gene and an operator disposed between the 5′ portion and the 3′ portion is introduced into a cell containing the target gene. After integration of the operator, expression of the target gene is under the control of the operator. For example, in some embodiments, the operator may be recognized by a transcriptional repressor. Numerous operators recognized by transcriptional repressors, such as the operators described herein, including the lac operator, the tet operator, the xyl operator, λc1 operator, the mal operator, or the trp operator, may be used.

After insertion of the operator in the target promoter or gene via homologous recombination, the extent of proliferation of cells in which the operator has been inserted under conditions in which the promoter is active at a first level is compared to the extent of proliferation of the cells under conditions in which the promoter is active at a second level which is lower than the first level. If the candidate gene is required for proliferation, the extent of proliferation of the cells grown under conditions where the promoter is active at the higher level will be greater than the extent of proliferation of cells grown under conditions where the promoter is active at the lower level.

The operator may be integrated at any location in which the activity or level of the gene product encoded by the target gene is modulated by the operator. For example, the operator may be integrated at any location in which expression of the target gene will be regulated by the effector which acts on the operator. For example, if the operator is integrated into a bacterial cell, the operator may be placed upstream of the −35 box, between the −35 box and −10 box, or downstream of the −10 box (e.g.overlapping the transcription start site). In some embodiments, the operator is integrated so as to maintain the appropriate relationships on the DNA helix between the transcription factors which direct expression from the promoter which is placed under the control of the operator.

As discussed above, preferably the cells into which the operator is to be integrated have an enhanced frequency of homologous recombination. In some embodiments, the operator may be introduced into the cell as linear double stranded DNA. Such DNA may be generated by an amplification reaction, such as a PCR reaction. In some aspects of this embodiment, the PCR reaction is performed on a linearized plasmid. In such embodiments, the cells may constitutively or conditionally express the λ Red recombinase system, comprising the Gam, Exo, and Bet proteins or an analogous system from another phage. Alternatively, the cells may comprise the recBCD and sbcA mutations, recBC and sbcA mutations or analogous mutations. In some embodiments, the cells constitutively or conditionally express the recE and recT proteins or proteins with analogous functions. In some embodiments, the cells comprise the recBCD and sbcA mutations, recBC and sbcA mutations, or analogous mutations, and constitutively or conditionally express the recE and recT proteins, or analogous proteins.

In some embodiments, the operator may be introduced into the cells as linear single stranded DNA. Such DNA may be synthesized in vitro or generated by sonication of a circular single stranded nucleic acid such as an M13 construct comprising the operator insertion construct. In some embodiments, the cells may constitutively or conditionally express the Bet protein of the λ Red recombinase system or an analogous protein from another phage. In still further embodiments, the expression of the proteins in the λ Red recombinase system, the Bet protein from the λ Red recombinase system, or analogous proteins from other phage may be conditional.

In some embodiments, the cells may be any of the cells described above which have an increased frequency of homologous recombination or any other cells having an increased frequency of homologous recombination which are familiar to those of skill in the art.

In some embodiments, the 5′ and 3′ portions of the linear operator insertion construct which have homology to the promoter region of the target gene or the target gene may have any length which permits homologous recombination to occur. Accordingly, in some embodiments, the 5′ and 3′ regions of the construct which have homology to the promoter region of the target gene or the target gene, may each be more than 120 nucleotides in length, at least 120 nucleotides in length, at least 100 nucleotides in length, at least 90 nucleotides in length, at least 80 nucleotides in length, at least 70 nucleotides in length, at least 60 nucleotides in length, at least 50 nucleotides in length, at least 40 nucleotides in length, at least 30 nucleotides in length or less than 30 nucleotides in length. For example, in some embodiments, the 5′ and 3′ regions of the construct which have homology to the target promoter or gene may be about 30 nucleotides in length.

Alternatively, in some embodiments, the operator insertion construct may be a single stranded nucleic acid, such as a synthetic oligonucleotide. Preferably, the single stranded nucleic acid is introduced into cells constitutively or conditionally expressing a protein which stabilizes single stranded DNA, such as the λ Bet protein or an analogous protein, as described in Ellis et al., PNAS 98: 6742-6746, 2001, the disclosure of which is incorporated herein by reference in its entirety. After introduction of the operator insertion construct into a suitable cell, cells in which the operator has integrated by homologous recombination are identified. The cells may be identified by performing an amplification reaction or Southern blot. Alternatively, to identify cells in which expression of a gene required for proliferation is regulated by the inserted operator, cells which grow well under conditions in which the promoter which is regulated by the operator is active at a higher level but not under conditions in which the promoter is active at a lower level are identified. For example, cells which grow on media containing an inducer which increases the level of transcription from the promoter but not on media in which transcription is repressed may be identified. In some embodiments, the operator may be the lac operator and cells which grow on media containing IPTG but not on media lacking IPTG may be identified. In some embodiments, the operator may be the tet operator and cells which grow on media containing tetracycline but not on media lacking tetracycline may be identified. In some embodiments, the operator may be the xyl operator and cells which grow on media containing xylose but not on media lacking xylose may be identified. In some embodiments, the operator may be the mal operator and cells which grow on media containing maltose but not on media lacking maltose may be identified. In some embodiments, the operator may be the trp operator and cells which grow on media lacking tryptophan but not on media containing tryptophan may be identified. In some embodiments, the operator may be the λc1 operator and cells which grow at the premissive temperature but not at the restrictive temperature may be identified.

The operator insertion method provides a fast and inexpensive way to engineer directed insertions into the chromosome. In some embodiments, the operator insertion construct may lack a nucleic acid encoding an identifiable or selectable marker. The method allows insertions of only the desired nucleic acid sequence without the necessity of introducing additional nucleic acid sequences such as sequences encoding identifiable or selectable markers. In addition, in embodiments where the native promoter is placed under the control of an operator, the expression level under conditions in which the promoter is not repressed or activated will be the physiological level.

The operator insertion method may be performed in any desired prokaryotic organism. For example, in some embodiments, the method may be practiced in Gram-positive bacteria. In certain embodiments, the cell to be used with any of the methods described herein is selected from the group consisting of Bacillus anthracis, Clostridium botulinum, Clostridium difficile, Clostridium perfringens, Clostridium teteni, Corynebacterium diptheriae, Enterococcus faecalis, Enterococcus faecium, Lactococcus lactis, Listeria monocytogenes, Mycobacterium leprae, Mycobacterium tuberculosis, Nocardia asteroides, Staphylococcus aureus, Staphylococcus epidermidis, Staphylococcus xylosis, Streptococcus pneumoniae, Streptococcus mutans or any species falling within the genera of any of the above species.

In some embodiments, the operator insertion method is conducted in cells which have an increased frequency of homologous recombination. For example, in some embodiments, the method is conducted in cells which carry the sbcA and recBC mutations, or in cells having mutations which confer similar phenotypes. In other embodiments, the cells may constitutively or conditionally express one or more proteins in the λ Red recombination system. For example, in some embodiments the cells may constitutively or conditionally express the λ Gam, Bet, and Exo proteins or other proteins having similar functions. In other embodiments, the cells may constitutively or conditionally express the λ Bet protein or a protein having a similar function.

In some embodiments, the promoter region for a target gene/operon is identified either experimentally or bioinformatically. An oligonucleotide is designed which matches at least 90, at least 70, at least 60, or at least 50 residues of the promoter region. An operator is engineered to be present in the middle of the oligonucleotide sequence. Thus, the operator will have at least 45, at least 35, at least 30, or at least 25 nucleotides of homology to the target region on each side. Examples of operators include the lac, tet, xyl, mal, trp, or λc1 operators, though any other operator known in the art can function. The oligonucleotide can be designed such that the operator is inserted: i) upstream of the −35 box, ii) in between the −35 and the −10 box, or iii) downstream of the −10 box.

In some embodiments, the oligonucleotides are introduced into cells that conditionally or constitutively express the λ Red (or homologous phage) recombination system, which consists of the λ Gam, Exo, and Bet proteins (Ellis et al., PNAS 98:6742-6746 (2001)). This recombination system efficiently recombines oligonucleotide DNA into the bacterial chromosome. Specifically, in some embodiments, the cells constitutively or conditionally express the λ Bet protein, which is a single strand binding protein that promotes annealing of complementary single strand and can mediate strand exchange, or another protein with an analogous function. Transformants are then plated on non-selective plates. To ascertain if a transforming is a recombinant, it is replica plated on plates that contain and lack the appropriate inducer. The plates are screened for an expected phenotype. For example, if the xyl operator is inserted downstream of the −10 box of an essential gene, then one would predict that recombinants would only be able to grow in the presence of inducer (xylose). For genes of known or expected phenotypes, e.g., those resulting in growth, transformants could be rapidly screened by plating on inducing (de-repressing) plates and then replica plating transformants on plates containing or lacking inducer. Those transformants that do not grow in the absence of inducer would contain the desired operator insertion.

Recombinants could also be verified by Southern or PCR analysis. This method is amenable to high throughput and thus many targeted recombinants could be constructed in a short time.

The operator insertion method also has the advantage of being portable to other bacteria by conditionally inducing the expression of the λ Bet protein. Additionally, this method could be employed to test if a gene is essential by screening for conditional growth, as discussed herein.

In some embodiments, the operator insertion method is conducted in cells which have an increased frequency of homologous recombination. For example, in some embodiments, the method is conducted in cells which carry the sbcA and recBC mutations, or in cells having mutations which confer similar phenotypes. In other embodiments, the cells may constitutively or conditionally express one or more proteins in the λ Red recombination system. For example, in some embodiments the cells may constitutively or conditionally express the λ Gam, Bet, and Exo proteins or other proteins having similar functions. In other embodiments, the cells may constitutively or conditionally express the λ Bet protein or a protein having a similar function.

In some embodiments, a double stranded operator insertion construct is introduced into a cell comprising the recBCD and sbcA mutations, recBC and sbcA mutations or mutations with analogous consequences. The double stranded operator insertion construct may or may not comprise a gene encoding a selectable or identifiable marker, but in preferred embodiments the double stranded operator insertion construct includes a gene enocding a selectable or identifiable marker. In other embodiments, a double stranded operator insertion construct is introduced into a cell constitutively or conditionally expressing the recE and recT proteins or proteins with analogous functions. In still other embodiments, a double stranded operator insertion construct is introduced into a cell constitutively or conditionally expressing the λ Gam, Bet, and Exo proteins or other proteins having similar functions. In such embodiments, cells in which the operator insertion construct has integrated are identified by obtaining colonies which express the identifiable marker or by growing the cells in appropriate selective media. If desired, homologous integration may be confirmed by performing a Southern blot or amplification reaction using a probe or primers which will yield a known result if homologous recombination has occurred.

In other embodiments, a single stranded operator insertion construct is introduced into the cell. Preferably, the single stranded operator insertion construct does not comprise a gene which encodes an identifiable or selectable marker, but in some embodiments a gene which encodes an identifiable or selectable marker may be included in the construct. In some embodiments, the single stranded nucleic acid may be generated by in vitro synthesis. In other embodiments, a circular single stranded DNA, such as M13, comprising the operator insertion construct may be fragmented by sonication and the fragments may be introduced into the cell. In still other embodiments, a single stranded operator insertion construct is introduced into a cell constitutively or conditionally expressing the λ Bet protein, the Bet and Gam proteins, the Bet, Gam and Exo proteins or other proteins having similar functions. Cells in which integration has occurred at the desired location are identified by performing colony hybridization, amplification reactions, or other methods familiar to those skilled in the art.

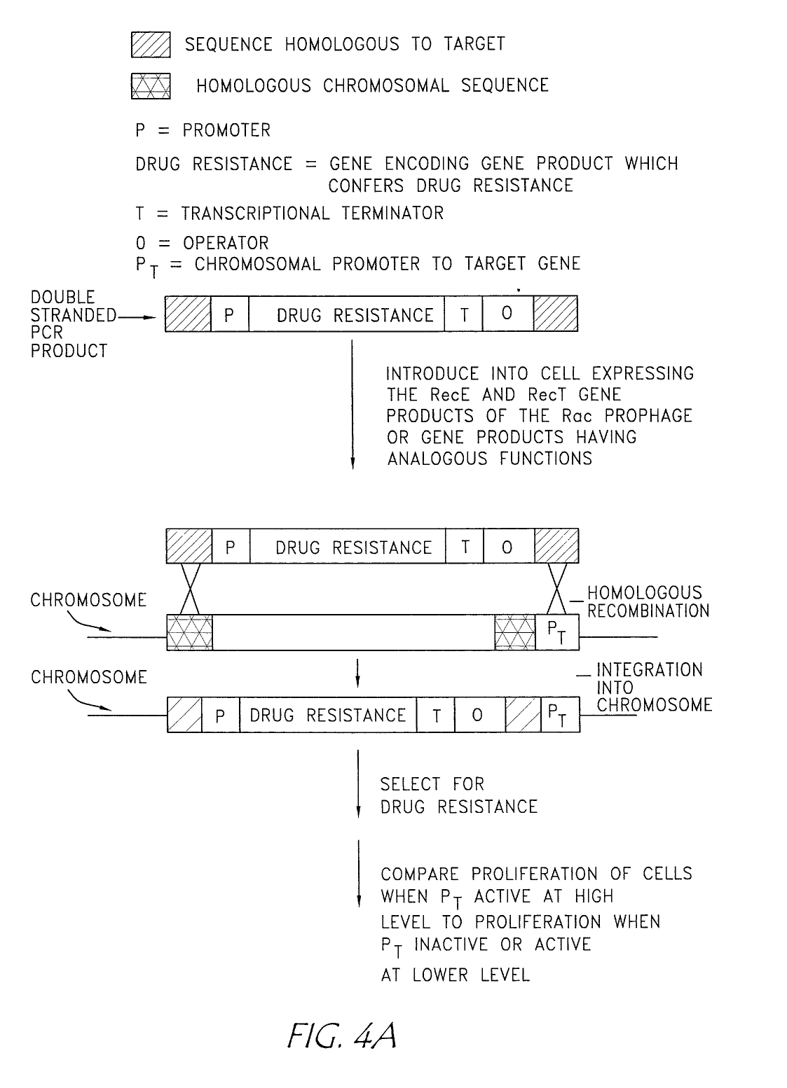

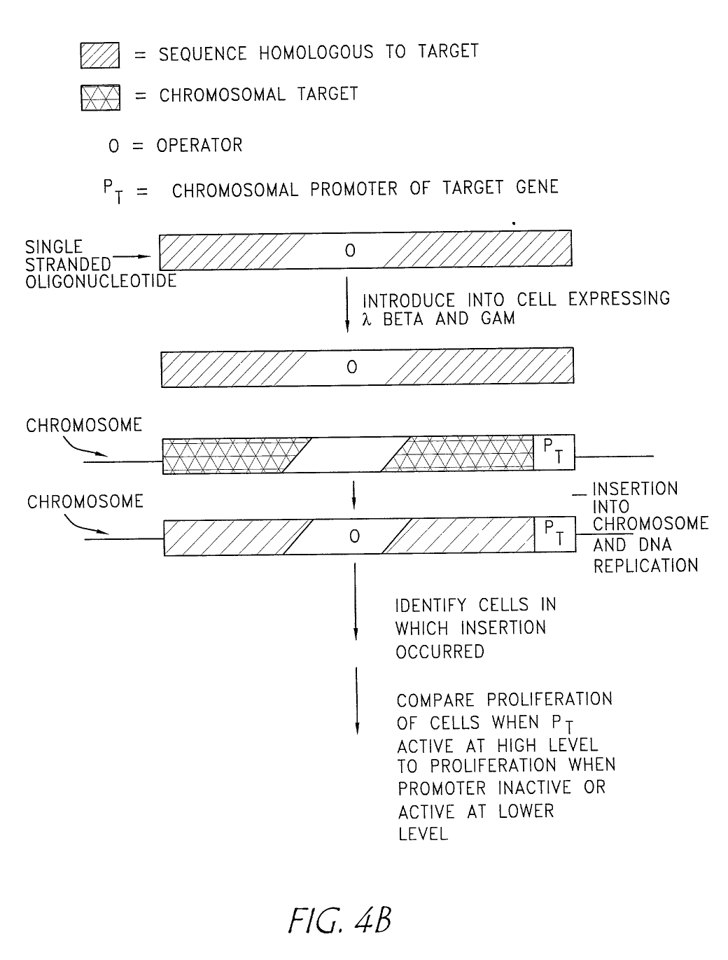

FIG. 4A illustrates one embodiment of the operator insertion method in which a linear double stranded regulatory element insertion construct comprising a nucleic acid encoding a selectable marker, a transcriptional terminator, and an operator is inserted into the chromosome of cells expressing the RecE and RecT gene products of the Rac prophage or gene products having analogous functions. FIG. 4B illustrates one embodiment of the operator insertion method in which a linear single stranded regulatory element insertion construct comprising a 5′ portion homologous to a target sequence, a 3′ portion homologous to a target sequence and an operator is inserted into the chromosome of a cell expressing the λ Beta and Gam proteins.

As discussed below, the operator insertion method may also be used to obtain cells in which the level or activity of a gene product required for proliferation is reduced. Such cells may be used in cell-based assays to identify compounds which inhibit proliferation.

In still another embodiment, a vector containing an outward-directed inducible promoter is randomly integrated into the genome of bacteria and clones containing these promoters that exhibit a reduction in viability and/or cell growth are identified. This technique is referred to as Random Insertional Cis-Antisense (random promoter insertion). This strategy involves the use of constructs that contain an inducible promoter (e.g., one of the promoters described herein) and a selectable marker to randomly integrate into the genome of a bacteria. When such constructs are integrated into or near an essential gene, induction of transcription from the inducible promoter will frequently reduce or eliminate normal expression of the essential gene. This event is particularly likely if the vector integrates such that transcription from the inducible promoter is in an antisense orientation relative to normal transcription of the endogenous gene. The integration in such cases is likely to be 3′ to the gene, within the 3′ untranslated region of the gene, within the coding region of the gene, or in the case of eukaryotes, within an intron of the gene. Induction of transcription in such integrants will dramatically interfere with normal sense transcription of the gene.

Since the expression of essential genes is required for proliferation of the organism, when such integration events occur within or near an essential gene, induction of transcription will be lethal or will severely impair proliferation of the organism. Such conditional lethal or conditional growth clones can be readily isolated and the genes into which the vector has integrated can be determined by the cloning and sequencing techniques described herein. In this manner, the random promoter insertion method can be used to discover essential bacterial genes.

Dominant negative mutants are another class of conditional lethal mutants that can be identified by using the random promoter insertion technique. These mutants can be generated when the vector integrates such that transcription from the inducible promoter is in the sense orientation relative to normal transcription of the endogenous gene. In this case the integration site can be 5′ to the gene, within the 5′ untranslated region of the gene, within the coding region of the gene, or in the case or eukaryotes, within an intron of the gene. Because the dominant negative mutants will involve certain classes of essential genes, the dominant negative mutants are useful for identifying a subset of essential genes. Such conditional dominant negative mutants can also be employed in a cell-based assay.

By one approach, a vector having the outward-directed inducible promoter is linearized by cutting with a restriction endonuclease and the linearized vector is transformed into bacteria. Sites of integration will be random but in many cases, the inducible promoter is oriented such that it produces an antisense RNA. Transformants are robotically picked, grown, and gridded onto inducing and non-inducing media (e.g., media having and lacking xylose, media having and lacking tetracycline, and media having and lacking maltose). The gridded plates are then screened for transformants that exhibit a growth defect on inducing media relative to the phenotype on non-inducing media. Transformants that have a reduced viability and/or cell growth in the presence of the inducer harbor a promoter that has integrated proximal to an essential gene. Once identified, the clone is sequenced to determine the identity of the essential gene. The genes that are affected by the random promoter insertion either directly or indirectly (e.g., by interacting with the RNA or protein that corresponds to the gene) causing a reduction in viability and/or cell growth are used as drug discovery targets for high throughput drug screening.

In some embodiments of the present invention, complementary to genes required for the proliferation of a host organism from which the antisense molecules were originally obtained may be used to identify homologous antisense nucleic acids and the coding nucleic acids complementary thereto from cells or microorganisms other than the original host organism, to inhibit the proliferation of cells or microorganisms other than the original host organism by inhibiting the activity or reducing the amount of the identified homologous coding nucleic acid or homologous polypeptide in the cell or microorganism other than the original host organism, or to identify compounds which inhibit the growth of cells or microorganisms other than the original host organism as described below. For example, antisense nucleic acids complementary to proliferation-required genes from original host organism may be used to identify compounds which inhibit the growth of Gram-positive organisms such as Bacillus anthracis, Clostridium botulinum, Clostridium difficile, Clostridium perfringens, Clostridium teteni, Corynebacterium diptheriae, Enterococcus faecalis, Enterococcus faecium, Lactococcus lactis, Listeria monocytogenes, Mycobacterium leprae, Mycobacterium tuberculosis, Nocardia asteroides, Staphylococcus aureus, Staphylococcus epidermidis, Staphylococcus xylosis, Streptococcus pneumoniae, Streptococcus mutans or any species falling within the genera of any of the above species.

In one embodiment of the present invention, antisense nucleic acids complementary to the sequences identified as required for proliferation or portions thereof are operably linked to a regulatable fusion promoter, such as one of the fusion promoters described herein, contained in a vectors capable of replicating within a species other than the species from which the proliferation-required sequences were obtained. Expression vectors which contain a fusion promoter and one or more origins of replication that are functional in one or more organisms, such as the expression vectors described herein, may be used. For example, the vector may be capable of replicating and/or producing transcripts in one or more of the following Gram-positive organisms selected from the group consisting of Bacillus anthracis, Clostridium botulinum, Clostridium difficile, Clostridium perfringens, Clostridium teteni, Corynebacterium diptheriae, Enterococcus faecalis, Enterococcus faecium, Lactococcus lactis, Listeria monocytogenes, Mycobacterium leprae, Mycobacterium tuberculosis, Nocardia asteroides, Staphylococcus aureus, Staphylococcus epidermidis, Staphylococcus xylosis, Streptococcus pneumoniae, Streptococcus mutans or any species falling within the genera of any of the above species.

Following the subcloning of the antisense nucleic acids, which are complementary to proliferation-required sequences or portions thereof from the host organism from which the stabilized antisense nucleic acids were originally obtained, into an expression vector under control of a regulatable fusion promoter capable of replicating and producing stabilized transcripts in a second cell or microorganism of interest (i.e. a cell or microorganism other than the one from which the identified nucleic acids were obtained), the antisense nucleic acids are conditionally transcribed to test for bacterial growth inhibition. The coding sequences complementary to the antisense nucleic acids from the original host that, when transcribed, inhibit growth of the second cell or microorganism are compared to the known genomic sequence of the second cell or microorganism to identify the homologous gene from the second organism. If the homologous sequence from the second cell or microorganism is not known, it may be identified and isolated by hybridization to the proliferation-required sequence of interest from the original host organism or by amplification using PCR primers based on the proliferation-required nucleotide sequence of interest. In this way, nucleotide sequences which may be required for the proliferation of the second cell or microorganism may be identified. For example, the second microorganism may be selected from the group consisting of Bacillus anthracis, Clostridium botulinum, Clostridium difficile, Clostridium perfringens, Clostridium teteni, Corynebacterium diptheriae, Enterococcus faecalis, Enterococcus faecium, Lactococcus lactis, Listeria monocytogenes, Mycobacterium leprae, Mycobacterium tuberculosis, Nocardia asteroides, Staphylococcus aureus, Staphylococcus epidermidis, Staphylococcus xylosis, Streptococcus pneumoniae, Streptococcus mutans or any species falling within the genera of any of the above species.

The homologous nucleic acid sequences from the second cell or microorganism which are identified as described above may then be operably linked to a promoter, such as one of the regulatable fusion promoter described herein, in an antisense orientation and introduced into the second cell or microorganism. The techniques described herein for identifying genes required for proliferation may thus be employed to determine whether the identified nucleotide sequences from a second cell or microorganism inhibit the proliferation of the second cell or microorganism. For example, the second microorganism may be selected from the group consisting of Bacillus anthracis, Clostridium botulinum, Clostridium difficile, Clostridium perfringens, Clostridium teteni, Corynebacterium diptheriae, Enterococcus faecalis, Enterococcus faecium, Lactococcus lactis, Listeria monocytogenes, Mycobacterium leprae, Mycobacterium tuberculosis, Nocardia asteroides, Staphylococcus aureus, Staphylococcus epidermidis, Staphylococcus xylosis, Streptococcus pneumoniae, Streptococcus mutans or any species falling within the genera of any of the above species. If the stabilized antisense nucleic acid inhibits the proliferation of the second cell or microorganism, a cell-based assay, such as the one described herein, can be used to test and identify candidate antibiotic compounds.

In another embodiment of the present invention, screening of candidate antibiotic compounds can be performed directly by using the antisense molecule isolated from the original host organism. In this embodiment, an antisense nucleic acid comprising a nucleic acid complementary to the proliferation-required sequences from the original host organism or a portion thereof is transcribed in the second host from a regulatable fusion promoter such as one of the fusion promoters described herein. If the antisense molecule is transcribed so as to sufficiently alter the level or activity of a nucleic acid required for proliferation of the second host, the second host may be used directly in a cell-based assay, such as those described herein, to identify candidate antibiotic compounds.

The techniques for identifying drug discovery targets described above can be used to generate bacterial strains for high throughput drug screening methodologies. Because the promoters described herein can be tightly and/or finely regulated over a wide range of maximal and basal transcription levels, one can selectively synthesize a precise amount of transcript or peptide sufficient to sensitize the cells without achieving complete lysis of cell population. For example, bacterial strains comprising the fusion promoters described herein are created using the EGI, promoter replacement, operator insertion, and random promoter insertion techniques. By carefully adjusting the amount of inducer in the growth media, an optimal balance of slowed growth and sensitivity to compounds can be achieved. Libraries of test compounds are then screened against the sensitized cells, the effect on cell growth is monitored, and compounds that further reduce viability and/or cell growth are identified. Through subsequent rounds of drug design, medicinal chemistry, and analysis, optimal antibiotics are developed using this inventive method.

Other embodiments concern an in vivo system for determining whether a gene is required for infection of a host by a bacterium. Such a system can also be used for validating the drug discovery targets and lead compounds identified by the approaches described above. Accordingly, bacterial strains (e.g., including bacterial strains such as Bacillus anthracis, Clostridium botulinum, Clostridium difficile, Clostridium perfringens, Clostridium teteni, Corynebacterium diptheriae, Enterococcus faecalis, Enterococcus faecium, Lactococcus lactis, Listeria monocytogenes, Mycobacterium leprae, Mycobacterium tuberculosis, Nocardia asteroides, Staphylococcus aureus, Staphylococcus epidernidis, Staphylococcus xylosis, Streptococcus pneumoniae, Streptococcus mutans or any species falling within the genera of any of the above species) having essential genes under the control of the regulatable promoters described herein are created using the EGI technology, promoter replacement technology, operator insertion technology or the random promoter insertion technology and these bacteria are used to inoculate a suitable host animal (e.g., neutropenic mice). Two hours after inoculation, an experimental group of animals are either provided a solution containing inducer (if the EGI technology or random promoter insertion technology was used to create the compromised cells) or a solution containing a minimal amount of inducer (if the promoter replacement technology or operator insertion technology was used to create the compromised cells). Accordingly, the bacteria are induced to generate the transcript or peptide that interacts with the essential gene or gene product or transcription of an endogenous essential gene is reduced. Subsequently, the amount of viable bacteria remaining in the animal or tissue sample from the animal is determined. By using this approach, one can rapidly evaluate bacterial strains carrying essential genes that can be selectively downregulated in vivo. Additionally, one can use these animal models to screen lead compounds in vivo so as to identify new antibiotics.

### EXAMPLE 1

### Construction of Fusion Promoters Having a Single Operator

This example describes the construction of the fusion promters that can be used to drive transcription of an RNA and/or expression of a peptide or protein in bacteria in a tightly regulated fashion. Many of the promoters described herein can function in multiple bacterial species including Gram-positive organisms such as Staphylococcus and Enterococcus species.

For construction of the Xyl-T5 fusion promoter, which is functional, for example, in Staphylcoccus aureus, a S. aureus/E. coli shuttle vector, referred to as pRNLX2, was initially constructed. First, the multiple cloning site was removed from the S. aureus vector pRN5548, obtained from Richard Novick at the Skirball Institute at New York University, by digesting the vector with HindIII. The remaining vector sequence was self-ligated, which resulted in the formation of a new vector referred to as pRNHIII. Next, the pRNHIII vector was digested with SalI and the DNA was treated with Calf intestine alkaline phosphatase (CIP). Subsequently, the SalI linearized and CIP-treated pRNHIII vector was ligated to a 2.6 kb XhoI/SalI fragment of pLex5BA, which contains the plasmid origin and ampicillin resistance marker. (Diederich, L. et al., BioTechniques 16: 916-923 (1994), the disclosure of which is incorporated herein by reference in its entirety). This vector, referred to as pRNLX2, contains both a S. aureus and an E. coli origin of replication. Thus, the vector can replicate in both Gram-negative and Gram-positive bacteria.

A xyl expression system was then constructed using the pRNLX2 shuttle vector. First, the xyl repressor gene and promoter were obtained from the plasmid pWH942 using PCR with the flanking primers 5′TGGCATTCTACGACTATAAC 3′ (SEQ ID No.: 3) and 5′GCGAATTCTACATTTTAGTTGGTTAATTT 3′ (SEQ ID No.: 4). (Schnappinger, D. et al., FEMS Microbiology Letters 129: 121-128 (1995), the disclosure of which is incorporated herein by reference in its entirety). These primers contain EcoRI sites near their 5′ ends and result in a PCR amplicon with trimmable EcoRI sites. The amplicons were digested with EcoRI and, subsequently, ligated into the pRN5548 vector, which had been linearized by digestion with EcoRI and SwaI. The resulting plasmid, called pXylRN, was used as a source for the xyl fragment for the final construct. The xyl fragment was excised from pXylRN with EcoRI and PshAI and was ligated to pRNLX2 that had been linearized with EcoRI and PvuII. The structure of the resultant plasmid was confirmed by sequencing the insert from both sides.

In the final step, the xyl promoter from pKLO20 was removed and replaced by the Xyl-T5 promoter. The xyl promoter and operator of pKLO20 is flanked by an RsrII site at its 5′ end and the EcoR1site of the multiple cloning site at its 3′ end. The promoter and operator were removed by digesting the plasmid with RsrII and EcoRI. The larger vector fragment of this digestion was purified from the smaller promoter fragment by preparative gel electrophoresis and a pair of oligonucleotides were designed having the following sequences:

| | |
|---|---|
| 5′GACCGTCATAAAAAATTTATTTGCTTTCAGGAAAATTTTTCTGTATAATA | (SEQ ID No.:5) |
| GATTCAAGTTAGTTTGTTTATTAAATTAACCAACTAAAATGTAG3′ | |
| 5′AATTCTACATTTTAGTTGGTTAATTTAATAAACAAACTAACTTGAATCTAT | (SEQ ID No.:6) |
| TATACAGAAAAATTTTCCTGAAAGCAAATAAATTTTTTATGACG3′. | |

These oligonucleotides were annealed and, thereby, formed a double-stranded fragment. This fragment included: the RsrII sticky end (underlined), an AT rich region, the T5 promoter, the xylO, and the EcoRI sticky end (italicized). This fragment was then combined with the purified RsrII-EcoRI vector fragment of pKLO20 and ligated to form the pXyl-T5 vector.

To facilitate the construction of fusion promoters that function in Enterococcus faecalis, a reporter plasmid having a swapable promoter module located between a xyl repressor gene (xylR) and xyl operator (xylO) was designed. The Lactococcus lactis promoter probe vector pAK80 (described in Israelsen, et al. Appl. Environ. Microbiol. 61, 2540-47 (1995), the disclosure of which is incorporated by reference in its entirety), which contains replicons functional in both Escherichia coli (p15A origin) and Enterococcus faecalis (PCT 1138 replicon) as well as a selectable marker for erythromycin resistance (erm), was digested at the unique HindIII and BamHI polylinker sites just upstream from the promoterless Leuconostoc mesenteroides β-galactosidase reporter genes (lacL-lacM). After digestion, the 11 kb vetor fragment was purified from the 26 bp polylinker by preparative gel electrophoresis then ligated to a 1535 bp gel purified HindIII/BamHI fragment from pEP25 (SEQ ID NO: 7) which contains the CP25 promoter from Lactococcus lactis (SEQ ID NO.: 38) flanked upstream by the xylose repressor gene (xylR) and downstream by the xylose operator sequence (xylO). A portion of the resulting ligation mixture was transformed into competent Escherichia coli XL-1 Blue cells (Stratagene, La Jolla, Calif.) and 20 μl, 75 μl or 100 μl of the transformation mixture was plated on BYGT medium containing 20 μg/ml erythromycin. Isolated erythromycin-resistant transformants were picked and streaked to obtain single colony isolates. Plasmid DNA was then purified from representative single colony transformants. The presence of the chimeric plasmid pEPEF1 (SEQ ID NO.: 8) was confirmed by digesting purified plasmid preparations with HindIII/BamHI and detecting an excised 1535 bp fragment, which corresponds to the fragment containing the Xyl-CP25 fusion promoter, by gel electrophoresis.

FIG. 2 shows a region of pEPEF1 that contains the Xyl-CP25 fusion promoter. Immediately adjacent to the 5′ end of the CP25 promoter and just downstream of xylR lies a unique RsrII recognition site (element (a) in FIG. 2). A unique recognition site for XhoI (element (b) in FIG. 2) lies immediately adjacent to the 3′ end of the CP25 promoter just upstream from xylO. The placement of these two unique restriction sites allow the CP25 promoter to be removed from pEPEF1 and replaced with promoter sequences from Lactococcus or other microbial species. For example, the CP25 promoter can be replaced with promoters having SEQ ID NOs.: 36-45.

In one example, a pEPEF1 derivative was created by replacing the CP25 promoter with the P59 promoter from Lactococcus lactis. Two complementary DNA oligonucleotides corresponding to the sequence of the P59 promoter were synthesized (SEQ ID NOs.: 9 and 10). Each sequence included a 5′-overhang end complementary to either an RsrII or XhoI overhang.

| | |
|---|---|
| 5′GACCGACATTAAATTCTTGACAGGGAGAGATAGGTTTGATAGAATATAA | (SEQ ID NO.:9) |
| TAGTTGTC3′ | |
| 5′TCGAGACAACTATTATATTCTATCAAACCTATCTCTCCCTGTCAAGAATT | (SEQ ID NO.:10) |
| TAATGTCG3′ | |

The underlined nucleotides comprise the 5′-overhang of the RsrII recognition site whereas the italicized nucleotides correspond to the 5′-overhang of the XhoI recognition site. The double stranded P59 RsrII/XhoI fragment was constructed by combining an equimolar amount of SEQ ID NOs.: 9 and 10, heating the mixture to 90° C. to denature any secondary structure, and allowing the complementary sequences to anneal by slowly cooling the solution to room temperature. The double stranded P59 promoter sequence was then ligated into gel-purified, RsrII/XhoI-digested pEPEF1 reporter vector thereby creating the Xyl-P59 fusion promoter. This reporter vector was termed pEPEF1-P59.

It will be appreciated that any promoter sequence, including the promoters of SEQ ID NOs.: 36-45, can be synthesized to include an RsrII overhang at one end and a XhoI overhang at the other. Promoter sequences can be wildtype promoters known from Lactococcus lactis and other Gram-positive species or promoters that have been modified to alter their transcription efficiency in gram-positive organisms. Example 7 provides a description of promoters that have been modified to alter their transcription efficiency in Staphylococcus aureus or Enterococcus faecalis.

The next example describes the construction of fusion promoters that have multiple operators.

### EXAMPLE 2

### Construction of Fusion Promoters Having a Plurality of Operators

This example describes the construction of fusion promoters comprising a plurality of operators. Fusion promoters such as Xyl/Lac-T5 and Xyl/Lac-P59 can be constructed by using a cloning strategy that is similar to the promoter swaping procedure described in Example 1. It will be appreciated that this procedure may be used to generate fusion promoters having two or more operators selected from the group consisting of xylO, tetO, trpO, malO, λc1O and lacO.

The lac operon is contained in many vectors including pGEM-11Zf(−), which is commercially available from Promega Corp. The sequence of the pGEM-11Zf(−) vector can be found in the NCBI database, Accession number X65314, the disclosure of which is incorporated herein by reference in its entirety. The lac operator comprises the 17 bp sequence: (TTGTTATCCGCTCACAA) (SEQ ID No. 11), which can be inserted in place of the 17 bp intervening sequence between the −35 Box and −10 Box of a variety of promoters, including the promoters of SEQ ID NOs.: 26-35.

In one example, a Xyl/Lac-T5 promoter can be made by replacing the 17 bp sequence, which comprises element (3) in FIG. 1, with the 17 bp lac operator sequence. To make the Xyl/Lac-T5 fusion promoter, two oligonucleotides are synthesized and annealed to create a double stranded fragment. The two oligonucleotides are provided below:

| | |
|---|---|
| 5′GACCGTCATAAAAAATTTATTTGCTTTGTGAGCGGATAACAATTATA | (SEQ ID No.:12) |
| ATAGATTCAAGTTAGTTTGTTTATTAAATTAACCAACTAAAATGTAG3′ | |
| 5′AATTCTACATTTTAGTTGGTTAATTTAATAAACAAACTAACTTGAATCT | (SEQ ID No.:13) |
| ATTATAATTGTTATCCGCTCACAAAGCAAATAAATTTTTTATGACG3′. | |

The resulting double stranded fragment includes: the RsrII sticky end (underlined portion of SEQ ID NO.: 12), an AT rich region found to increase the level of transcription from the T5 promoter in Gram-positive bacteria, the T5 promoter, the lac operator (lacO), the xylose operator (xylO), and the EcoRI sticky end (italicized portion of SEQ ID NO.: 13). This double standed fragment can be combined with the purified RsrII/EcoRI vector fragment of pKLO20 and ligated to form the vector pXyl/LacT5.

In a similar example, a Xyl/Lac-P59 promoter can be constructed by replacing the 17 bp sequence between the −35 Box and −10 Box regions (nucleotides 18-34) of SEQ ID NO.: 40) of P59 with the 17 bp lac operator sequence (SEQ ID NO.: 11). To make the Xyl/Lac-P59 fusion promoter, two oligonucleotides are synthesized and annealed to create a double stranded fragment. The two oligonucleotides are provided below:

| | |
|---|---|
| 5′GACCGACATTAAATTCTTGACATTGTGAGCGGATAACAATAGAATATAA | (SEQ ID NO.:14) |
| TAGTTGTC3′ | |
| 5′TCGAGACAACTATTATATTCTATTGTTATCCGCTCACAATGTCAAGAATT | (SEQ ID NO.:15) |
| TAATGTCG-3′. | |

The underlined nucleotides comprise the 5′-overhang of the RsrII recognition site whereas the italicized nucleotides correspond to the 5′-overhang of the XhoI recognition site. The double stranded P59/Lac fusion promoter formed by the annealing of these two oligonucleotides is then ligated with purified, RsrII/XhoI-digested pEPEF1, which contains the xylO operator, to form the vector pEPEF1-Lac/P59.

It will be appreciated that there are many equivalent techniques that can be used to construct fusion promoters having a plurality of operators. It will also be appreciated that operators other than lacO and promoters other than those described herein may be used in multi-operator constructs. For example, the fusion promoters may comprise a plurality of operators selected from the group consisting of xylO, tetO, trpO, malO, λC1O or lacO. Furthermore, one of ordinary skill in the art will recognize that an operator may be positioned in a various locations with respect to the promoter provided that the operator retains sufficient ability to regulate transcription.

The next example describes the construction of expression vectors containing regulatable fusion promoters.

### EXAMPLE 3

### Multipurpose Expression Vectors Containing Fusion Promoters

In this section, the construction and use of expression vectors comprising fusion promoters is described. Because these expression vectors can accept a wide range of fusion promoters each having a range of transcriptional activity, these vectors are useful for finely modulating the amount of nucleic acid or polypeptide expressed inside a host cell throughout a wide range of concentrations.

It will be appreciated that a regulatable fusion promoter suite for use in expression vectors can be constructed which provides a wide dynamic range of both basal and maximal transcriptional activities. Each fusion promoter within this suite can be tightly and finely regulated over its own characteristic range of transcriptional activity. Furthermore, the regulatable fusion promoters within the suite can comprise different promoters operably linked to the same operators thus providing for a common means of regulation of the promoters within the suite. For example, the promoters within the set of regulatable fusion promoters within the suite can be linked to at least one operator contained in a promoterless expression vector that is capable of replicating in one or more Gram-positive organisms. In other embodiments, a promoter operator fusion construct may be inserted into the vector as a cassette. Accordingly, a regulatable fusion promoter having an appropriate basal level and/or maximal level of transcriptional activity can be selected from the suite for use in the appropriate application. As described below, fusion promoters can be quickly and easily exchanged in the expression vector as needed.

Fusion promoters prepared as described in the preceding sections can be incorporated into existing expression vectors using conventional techniques in molecular biology. Embodiments include, for example, expression vectors having promoters of SEQ ID NOs.: 36-45 linked to operators such as xylO, xylO/lacO or one or more operators including, but not limited to tetO, malO, lacO, trpO, and λc1O. In some embodiments, the expression vectors comprising the fusion promoters allow for the transcription and/or expression of a peptide protein or transcript in a plurality of Gram-positive and Gram-negative bacterial species (e.g., Escherichia coli Staphylococcus species, and Enterococcus species).

These vectors can also comprise any of the following elements: genes encoding assayable markers such as enzymes that produce detectable products, genes encoding selectable markers such as polypeptides that control drug resistance, one or more unique restriction sites positioned downstream of the promoter such that inserts cloned into the site can be transcribed from the promoter, one or more transcription terminators such as a rho independent or rho dependent terminators which are positioned downstream of the unique cloning sites so as to terminate transcription of the nucleic acid insert, an origin of replication functional in Gram-negative species such as Escherichia coli, and an origin of replication functional in Gram-positive species such as Staphylococcus or Enterococcus.

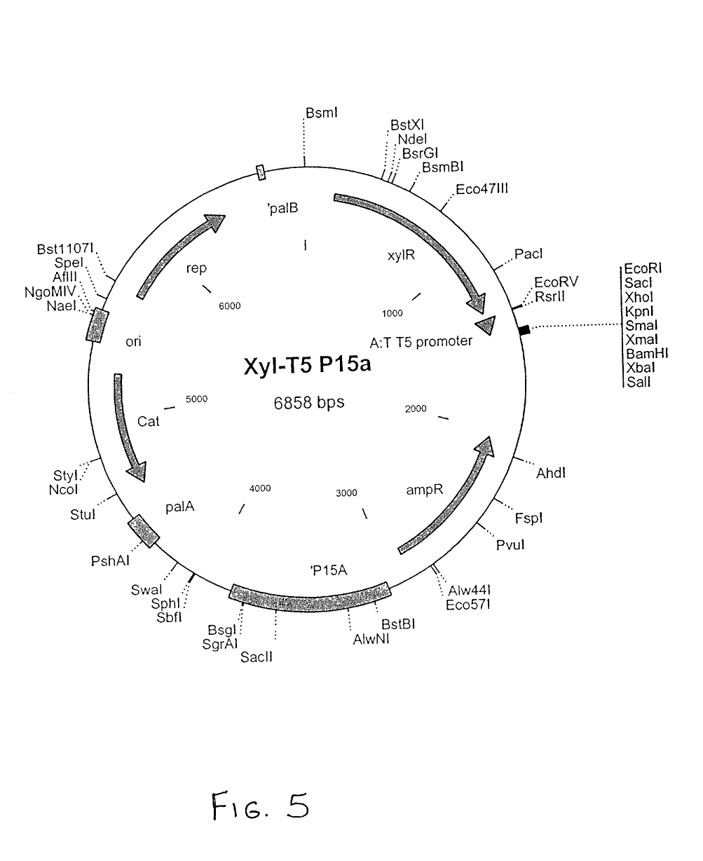

For example, pXyl-T5 P15a is an expression vector which comprises the Xyl-T5 fusion promoter (see FIG. 5). This vector is 6858 bp in length and can be used to transcribe RNA and/or express a peptide or protein in Gram-positive bacteria including, but not limited to, Staphylococcus species. This vector comprises the Xyl-T5 fusion promoter positioned between the xylR gene and a multiple cloning site having unique restriction sites (e.g., EcoRI, SacI, XhoI, KpnI, SmaI, XmaI, BamHI, XbaI, and SalI sites), the beta-lactamase gene from pLex5BA the P15a replicon (which maintains the vector at approximately 15 copies/cell in E. coli), and the pC194 Staphylococcus replicon (which includes palA, palB, the pc194 origin of replication, the rep gene, and a chloramphenicol acetyl transferase gene that imparts chloramphenicol resistance in Gram-positive bacteria).

The pXyl-T5 p15a vector was constructed from pXyl-T5 by replacing the E. coli ColE1 origin of pXyl-T5, which allows replication of the vector at 100-200 copies per cell, with the P15a origin of pACYC184, which allows replication of the vector at approximately 15 copies per cell in E. coli. (Chang, A. C. Y. and Cohen, S. N., J. Bacteriol. 134:1141-1156 (1978), the disclosure of which is incorporated herein by reference in its entirety). The ColE1 origin lies within two closely linked NotI sites. To obtain the Xyl plasmid having the ColE1 origin, the pXyl-T5 vector was digested with NotI and the larger vector fragment was purified by preparative gel electrophoresis. Next, a derivative of plasmid pACYC184, which includes the P15a origin as a similar NotI cassette, was digested with NotI and the smaller P15a-encoding fragment was gel purified. (Diederich, L. et al., BioTechniques 16: 916-923 (1994), the disclosure of which is incorporated herein by reference in its entirety). The two purified NotI fragments were combined and ligated to form the vector pXyl-T5 P15a. Table 1 lists the components of the pXyl-T5 P15a vector and their locations.

TABLE 1

| | Start | End | | |
|---|---|---|---|---|
| Type | Position | Position | Name | Description |
| Gene | 176 | 1327 | xylR | The gene encoding repressor |
| | | | | protein XylR |
| Promoter | 1360 | 1455 | A:T rich | The modified T5 promoter |
| | | | T5 | with upstream A:T rich box |
| | | | promotor | |
| Region | 1455 | 1494 | Multiple | Multiple cloning site includes |
| | | | Cloning | following unique restriction |
| | | | site | sites: EcoRI, SacI, XhoI, |
| | | | | KpnI, SmaI, XmaI, BamHI, |
| | | | | XbaI, SalI |
| Gene | 2903 | 2043 | ampR | The beta-lactamase gene from |
| | | | | pLex5BA |
| Region | 3037 | 3852 | P15a | A replicon that allows |
| | | | | maintenance of vector at |
| | | | | approximately 15 copies per |
| | | | | cell in E. coli hosts. Inserted |
| | | | | as NotI cassette at the Start |
| | | | | and End numbers of the |
| | | | | vector sequence. |
| Region | 4302 | 6659 | | S. aureus replicon from |
| | | | | pC194. Includes the five |
| | | | | features described below: |
| Region | 4462 | 4302 | C palA | With palB, imperfect direct |
| | | | | repeats |
| Gene | 5242 | 4592 | C Cat | Gram-positive Chloramphen- |
| | | | | icol acetyl transferase gene- |
| | | | | imparts chloramphenicol |
| | | | | resistance in S. aureus. |
| Region | 5562 | 5399 | C ori | pC194 origin of replication |
| Gene | 5699 | 6388 | rep | gene for rep protein |
| Region | 6659 | 6626 | C palB | |

A plasmid map of pXyl-T5 P15a is provided in FIG. 5. The nucleotide sequence of pXyl-T5 P15a is provided in SEQ ID NO.: 16.

It will be appreciated that corresponding expression vectors which include fusion promoters comprising either single or multiple operators can be constructed using conventional techniques in molecular biology. For example, pXyl/Lac-T5 p15a can be constructed by digesting pXyl-T5 p15a with RsrII and EcoRI so as to remove the Xyl-T5 fusion promoter. After purification, the linear vector fragment is ligated to an RsrII/EcoRI fragment comprising the pXyl/Lac-T5 promoter the contruction of which was described in Example 2.

In another example, expression vectors functional in both Escherichia coli and Enterococcus faecalis were constructed by replacing the lacL-lacM reporter genes of pEPEF1 with the multiple cloning site (MCS) and rrnBtlt2 terminator of pLexP20. Specifically, pEPEF1 was digested with SmaI and SalI to remove the 3808 basepair fragment containing the lacL-lacM reporter genes. Following digestion, the large vector fragment was gel purified, the SalI overhanging end was made blunt with T4 DNA polymerase, and the fragment was circularized by ligating the blunt ends. The resulting plasmid, termed pEPEF2, was then digested with BamHI, gel purified, and blunted with T4 DNA polymerase. The ClaI/BstB1 fragment containing the MCS and rnnBtlt2 terminator was removed from pLexP20, gel purified, blunted using T4 DNA polymerase, then ligated to the blunt ended pEPEF2. A portion of the resulting ligation mixture was transformed into competent Escherichia coli and aliquots of the transformation mixture were plated on medium containing 20 μg/ml erythromycin. Isolated erythromycin-resistant transformants were picked and streaked to obtain single colony isolates. Plasmid DNA was then purified from representative single colony transformants. The presence and orientation of the MCS/terminator fragment was determined by PCR and DNA seqeuncing, respectively. A plasmid comprising the MCS/terminator fragment oriented such that the MCS was located adjacent to xylO was termed pEPEF3 (SEQ ID NO.: 17).

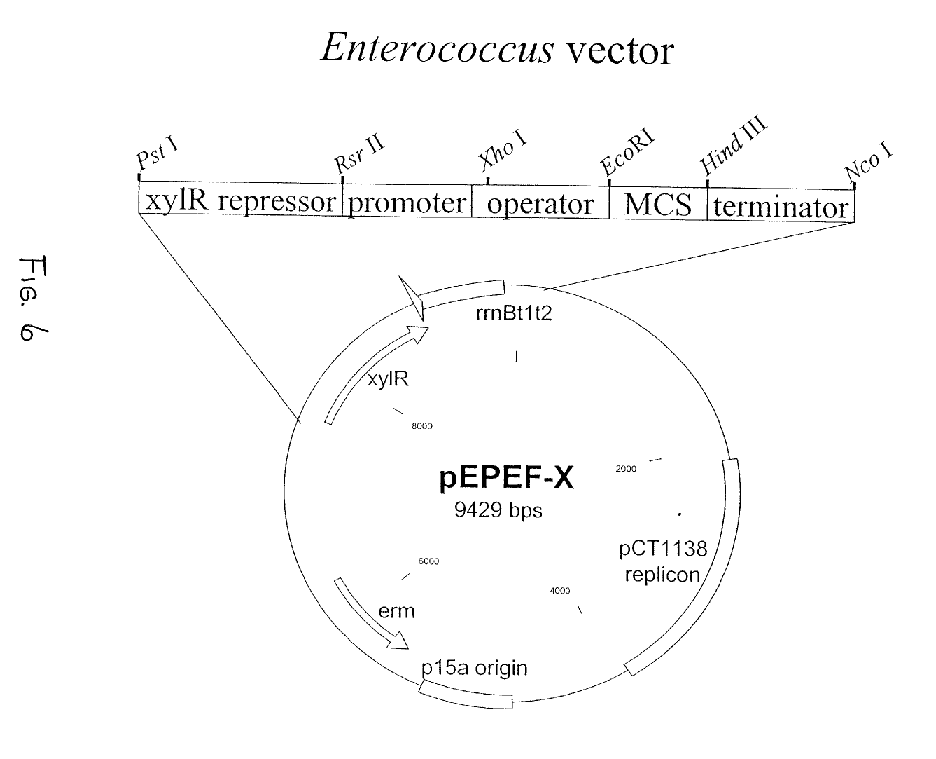

It will be appreciated that expression vectors such as those described above can be used to construct derivative expression vectors which include fusion promoters comprising either single or multiple operators. For example, the CP25 promoter can be removed from pEPEF3 by digestion of this vector with RsrII and XhoI. After purification, the linear vector fragment is ligated to an RsrII/XhoI fragment comprising P59. A pEPEF based expression vector having a generic promoter, termed pEPEF-X is shown in FIG. 6. Accordingly, many expression vectors derived from either pXyl-T5 or pEPEF3 can be constructed by exchanging the promoter sequence contained therein with the desired promoter sequence. In some embodiments, an expression vector is constructed comprising a promoter selected from SEQ ID NOs.: 36-45. pEPEF-X based expression vectors containing the Lactococcus lactis derived promoter sequences described herein, include pEPEF9 (SEQ ID NO.: 46), pEPEF14 (SEQ ID NO.: 47), pEPEF14* (SEQ ID NO.: 48), P1P2/EF14 (SEQ ID NO.: 49), pEPEF18 (SEQ ID NO.: 50), pEPEF20 (SEQ ID NO.: 51), and pEPEF22 (SEQ ID NO.: 52).

Several methods of transcribing an RNA and/or expressing a peptide or protein are also embodiments included herein. More specifically, the fusion promoters described herein can be used to transcribe a sense or anti-sense RNA or can be used to express a recombinant peptide or protein in a cell.

The next example describes experiments that were performed to evaluate the ability of the Xyl-T5 promoter to transcribe RNA.

### EXAMPLE 4

### Activity of Fusion Promoters by Real-Time RT-PCR

To analyze the strength of the Xyl-T5 promoter that comprised the T5 promoter operably linked to an AT Box and an xylO operator, transcript accumulation experiments were conducted using real-time RT-PCR (Reverse Transcriptase Polymerase Chain Reaction). RT-PCR detects a specific RNA by PCR amplification of a reverse transcriptase synthesized cDNA copy of the RNA. With real time RT-PCR, the accumulation of PCR amplified DNA is measured at each amplification step using a specific fluorogenically labeled oligonucleotide probe. The probe anneals to the newly formed cDNA product and is subsequently cleaved by the 5′ nuclease activity of the Taq DNA Polymerase during DNA amplification. Cleavage of the probe results in an increase in fluorescent dye signal, which is used to monitor accumulation of the specific PCR product at each PCR amplification step. The PCR cycle time at which logarithmic amplification is reached is proportional to the initial amount of RNA template in the reaction.

The levels of transcription from the Xyl-T5 promoter were evaluated in the following experiment. Fresh overnight colonies of Staphylococcus aureus RN4420 containing plasmid a plasmid having the Xyl-T5 promoter were added to 10 ml of Luria Broth (LB) that was supplemented with 15 μg/ml chloramphenicol (LB CM15). The cells were grown at 37° C. with shaking until early log phase (OD600 between 0.1 and 0.2) at which point they were diluted 1/10 into pre-warmed LB CM15. After one hour of growth at 37° C. with shaking, three 1 ml aliquots of cells were pelleted and quick frozen on dry ice. The remaining suspension was split into two flasks. Xylose was added to a final inducer concentration of 2.0% in one flask, whereas the second flask remained non-induced (control). The resulting transcript is approximately 300 nucleotides in length and corresponds to vector sequence.

Immediately after xylose induction (2 minute time point) and at 10, 20, 30 and 60 minute time points, (three) 1 ml aliquots of cells were pelleted from each flask (with and without xylose) and quick frozen on dry ice. Cell pellets were stored at −80° C. Total RNA was purified from each sample using a Qiagen 96 well RNeasy kit. Approximately, 1 ng of RNA (two samples for each time point) was used as template for RT-PCR with an oligonucleotide primer set (forward and reverse primers, and a fluorogenic probe) for specific detection of the 3′ tail of the Xyl-T5 transcript. The reaction consisted of 300 nM each of the forward and reverse primers; 100 nM fluorogenic probe; 0.75 U AmpliTaq Gold (PE Biosystems); 7.5 U MultiScribe Reverse Transcriptase (PE Biosystems), 1× Buffer A (PE Biosystems); 5.8 mM MgCl, and 333 nM of each dNTP. The RT-PCR conditions were 48° C. for 30 minutes, 95° C. for 10 minutes, followed by 40 cycles of 95° C. for 15 seconds and 60° C. for 1 minute. RT-PCR reactions and detection were done in an ABI Prism 7700 Sequence Detection System.

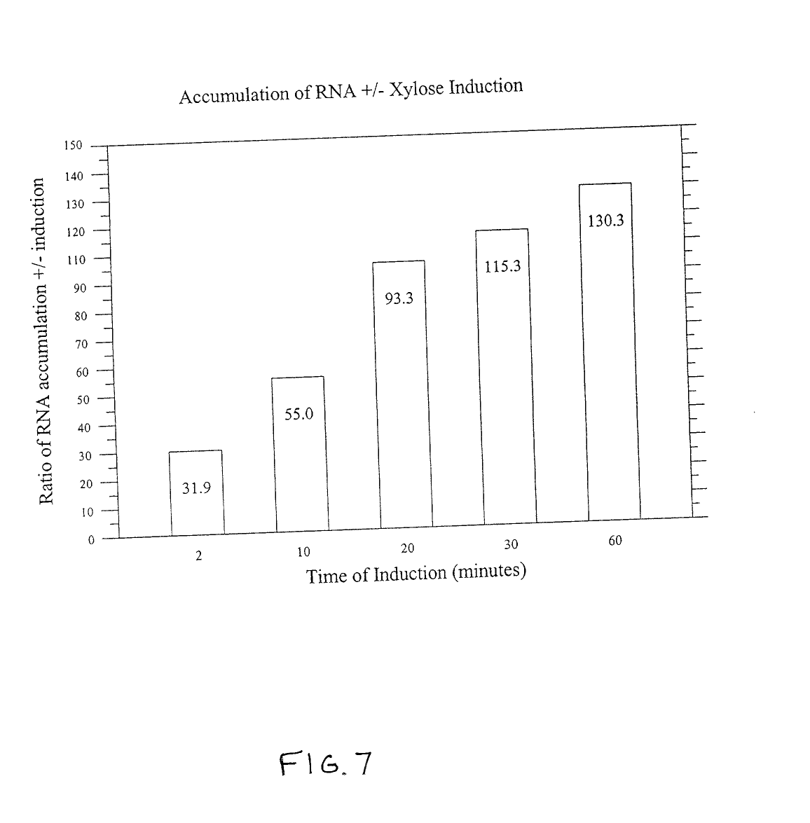

The results of the experiment described above are shown in FIG. 7. Within 2 minutes of xylose induction, greater than a 30 fold excess of RNA was detected, as compared to the non-induced controls. At 60 minutes, greater than a 130 fold excess of RNA was detected in the xylose induced samples, as compared with non-induced bacteria. This example demonstrates that the fusion promoters described herein can be used to efficiently transcribe an RNA. Further, the data described herein verify that in the absence of xylose or an analog thereof Xyl-T5 promoter-driven transcription can be effectively repressed.

It will be appreciated that the fusion promoters described above can be used to construct a regulatable fusion promoter suite which provides a wide dynamic range of both basal and maximal transcriptional activities. Each fusion promoter within this suite can be tightly and finely regulated over its own characteristic range of transcriptional activity. Furthermore, the regulatable fusion promoters within the suite can comprise different promoters operably linked to the same operators thus providing for a common means of rapidly regulating the promoters within the suite. In some embodiments, the promoters within the set of regulatable fusion promoters within the suite can be linked to at least one operator contained in a promoterless expression vector that is capable of replicating in one or more Gram-positive organisms. Accordingly, a regulatable fusion promoter having an appropriate basal level and/or maximal level of transcriptional activity can be selected from the suite for use in the appropriate application.

The example below demonstrates the relative strengths of the fusion promoters described herein.

### EXAMPLE 5

### Activity of Fusion Promoters by Expression of Reporter Genes

This example describes the use of reporter gene constructs to measure the activity of fusion promoters such as those constructed in Examples 1 and 2. In one experiment, the pXyl-T5 and pXyl-T5 P15a vectors were tested to determine the relative level of expression of a reporter gene in both Escherichia coli and Staphylococcus aureus. To test the ability of the Xyl-T5 promoter to induce transcription and express a heterologous polypeptide, a construct was made in which the reporter gene was operably linked to the Xyl-T5 promoter. A pXyl-T5 vector was digested with SacI and SalI, and the linearized vector was purified by preparative gel electrophoresis. The linearized vector fragment was then ligated with a fragment containing the reporter gene. This construct was named pXyl-T5-Reporter. The pXyl-T5-Reporter vector containing the reporter gene under the control of the Xyl-T5 promoter was then introduced into S. aureus and the cells were cultured in the presence and absence of 2.0% xylose. The transcription and translation of the gene product encoded by the reporter gene was evident when xylose was provided in the growth medium but in the absence of an agent that inhibits the binding of the xylose repressor to the xylose operator, production of the gene product encoded by the reporter gene was not detectable.

When the vector containing the reporter gene under the control of the Xyl-T5 promoter was transformed in Escherichia coli, it was noted that expression of the gene product encoded by the reporter gene was very high, and uncontrollable either in the presence or absence of xylose. This may be because Escherichia coli does not express the XylR repressor. In those embodiments where it is desirable that Escherichia coli serve as a surrogate host, pXyl-T5 p15a, which has a reduced copy number in Escherichia coli can be used.

To evaluate the effect of the P15a origin on the level of reporter gene expression in Staphylococcus aureus and Escherichia coli in the presence and absence of xylose, a construct similar to pXyl-T5-Reporter was made, using the pXyl-T5 p15a vector. This construct was denoted pXyl-T5 p15a-Reporter. A comparison of the induction of reporter gene expression of pXyl-T5-Reporter and pXyl-T5 p15a-Reporter showed that the two vectors functioned identically when transformed and expressed in Staphylococcus aureus, but the P15a variant showed a markedly reduced expression of the reporter gene when transformed in Escherichia coli. The demonstrates that the p15a origin of replication decreases reporter gene expression by reducing the copy number of the expression vector thereby reducing the number Xyl-T5 promoters in the cell.

Fusion promoters contained on pEPEF1 and its derivatives were evaluated in determine their strength in Enterococcus faecalis by measuring the β-galactosidase activity produced by expression of the lacL-lacM reporter genes under induced and uninduced conditions. In one experiment, derivatives of pEPEF1 were prepared by replacing CP25 with the Lactococcus lactis derived promoters P32 (SEQ ID NO.: 39), P59 (SEQ ID NO.: 40), or P1P2 (SEQ ID NO.: 42) as described in Example 1. pEPEF1 and the resulting derivatives were each separately transformed into electrocompetent Enterococcus faecalis and the cells were cultured in the absense and the presence of 5% xylose. The β-galactosidase activity produced by the expression of the reporter genes was measured essentially as described in Israelsen et al. Appl. Environ. Microbiol. 61:2540-47 (1995), the disclosure of which is incorporated herein by reference in its entirety, with the following modifications. After incubation with or without xylose for four hours, the cells were harvested by centrifugation then resuspended in 1 ml of Z buffer comprising 0.06 M Na₂HPO₄.7H₂O, 0.04 M NaH₂PO₄.H₂O, 0.01 M KCl, 0.001 M MgSO₄.7H₂O, 0.05 M β-mercaptoethanol, at pH 7.0, with 0.6 ml of glass beads (150-212 micron diameter). The cells were lysed using a bead beater at maximun speed for 1 minute. After centrifugation, the supernatant was removed, vortexed with one drop of 0.1% SDS and 2 drops of chloroform at high speed for 10 seconds, then incubated with 200 μl of a 4 mg/ml o-nitrophenyl-β-D-galactopyranoside (ONPG) solution at 37° C. After approximately 20 minutes, the reaction was stopped by adding 250 μl of 1M sodium carbonate. β-galactosidase activity was quantitated spectrophotometrically. All assays were performed in triplicate.

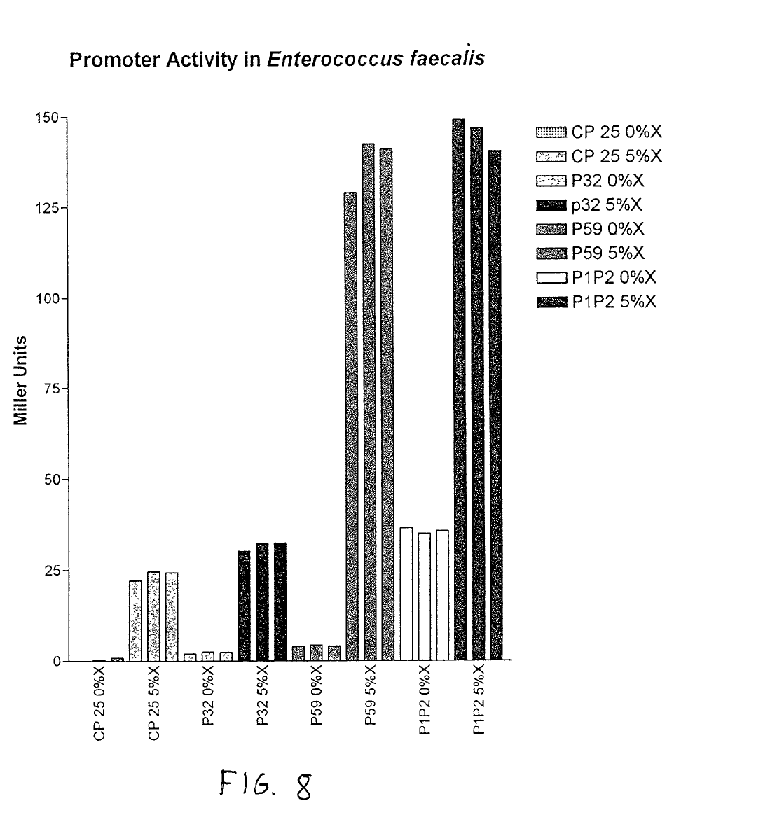

FIG. 8 displays the amount of β-galactosidase activity produced by each fusion promoter in Enterococcus faecalis in the presence and absence of xylose. Promoters CP25 and P32 both produce very little β-galactosidase activity in the absence of xylose; however, in the presence of 5% xylose, both of these promoters express enough β-galactosidase to produce approximately 25 Miller units of activity. The activities of both P59 and P1P2 are much greater than that of either CP25 or P32. In the presence of xylose, P59 and P1P2 each produce between 125 and 150 Miller units of β-galactosidase activity. In the absence of xylose, P59 is strongly repressed having less than 4% of its induced activity. By contrast, P1P2 produces about 20% of its induced activity even in the absence of xylose.

The above data shows that the fusion promoters described herein differ in the levels of transcription which they provide and the levels of transcription under non-inducing conditions. Some promoters are highly active under induced conditions whereas others show only a modest increase in activity upon induction. Furthermore, some promoters described herein may be strongly repressed in the absence of xylose whereas the activity of other promoters may only be partially inhibited.

It will be appreciated that the fusion promoters described above can be used to construct a regulatable fusion promoter suite which provides a wide dynamic range of both basal and maximal transcriptional activities. Each fusion promoter within this suite can be tightly and finely regulated over its own characteristic range of transcriptional activity. Furthermore, the regulatable fusion promoters within the suite can comprise different promoters operably linked to the same operators thus providing for a common means of regulation of the promoters within the suite. In some embodiments, the promoters within the set of regulatable fusion promoters within the suite can be linked to at least one operator contained in a promoterless expression vector that is capable of replicating in one or more Gram-positive organisms. Accordingly, a regulatable fusion promoter having an appropriate basal level and/or maximal level of transcriptional activity can be selected from the suite for use in the appropriate application.

In another experiment, the activity of the CP25 fusion promter was compared to the activity of Xyl-T5. To produce a pXyl-T5 p15a vector containing the lacL-lacM reporter, the purified 4 kb HindIII/SalI fragment of pAK80 which contains the lacL-lacM reporter construct was ligated into purified pXyl-T5 p15a which had been linearized using HindIII and SalI. The resulting constuct was termed pXyl-T5 p15a LacLM. To compare the activity of the T5 fusion promoter with that of CP25, pXyl-T5 p15a LacLM and pEPEF1 were transformed separately into Enterococcus faecalis and the cells were cultured in the presence of 1% and 5% xylose and in its absence. The β-galactosidase activity produced by the expression of the reporter genes was measured using the Miller assay as described above.

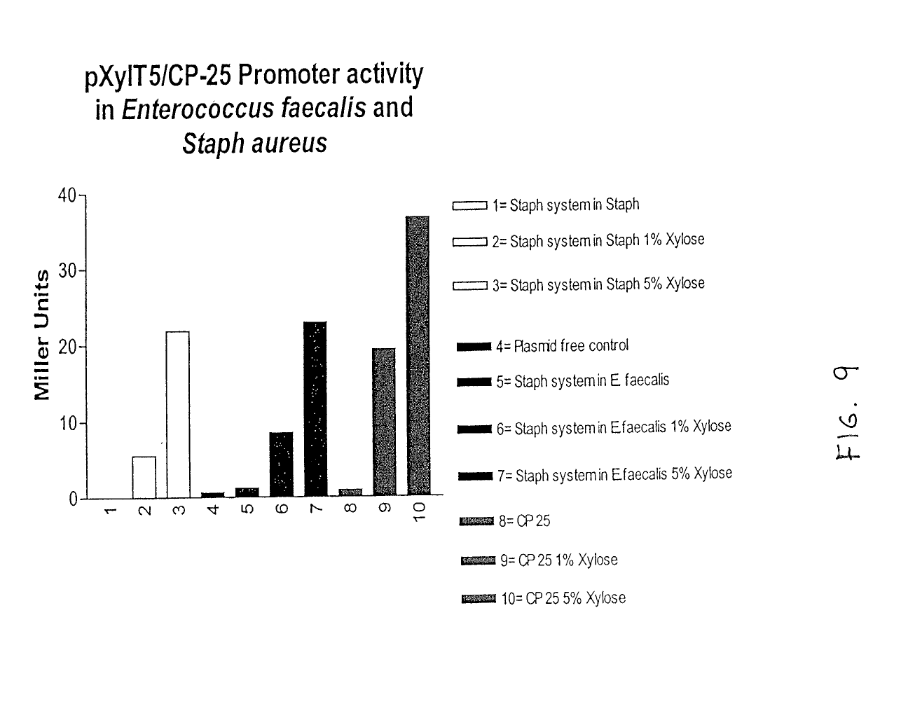

FIG. 9 displays the amount of β-galactosidase activity produced by each fusion promoter under induced and uninduced conditions. In the absence of xylose, both the fusion promoters are strongly repressed in Enterococcus faecalis. When induced with 1% xylose, CP25 has about twice the activity of Xyl-T5 in Enterococcus faecalis. At 5% xylose, CP25 produces about 35 Miller units of activity compared to about 22 Miller units by Xyl-T5.

To determine whether there is a difference in the level of gene expression from pXly-T5 p15a in Staphylococcus aureus and Enterococcus faecalis, pXyl-T5 p15a LacLM was also transformed into Staphylococcus aureus which was grown in the presence of 1% and 5% xylose and in its absensce. The β-galactosidase activity produced by the expression of the reporter genes was measured using the Miller assay as described above.

FIG. 9 shows that the activity of Xyl-T5 is similar in Staphylococcus aureus and Enterococcus faecalis. When induced with 5% xylose in each organism, the Xyl-T5 fusion produces appoximately 22 Miller units of β-galactosidase activity.

The above data shows that the fusion promoters disclosed herein have the ability to function in a variety of Gram-positive organisms and to maintain their characteristic activities when transferred from one host to another. This result is in constrast to the alteration in activity that is seen when the promoter is transferred to Gram-negative organisms. One example is the unregulated activity of Xyl-T5 in in Escherichia coli.

The example below demonstrates that the activity of the fusion promoters described herein can be finely regulated over a range of inducer concentrations. In other words, the activity of these fusion promoters is titratable.

### EXAMPLE 6

### Titratable Expression from Fusion Promoters

This example shows that the activity of promoters described herein is titratable with inducer thus allowing a reproducible, incremental modulation of promoter activity by altering the concentration of inducer. Each fusion promoter described herein possesses its own characteristic activity over a range of xylose concentrations. Within this range, the amount of RNA transcribed from a fusion promoter can be finely regulated in repsonse to the concentration of xylose used to induce the promoter.

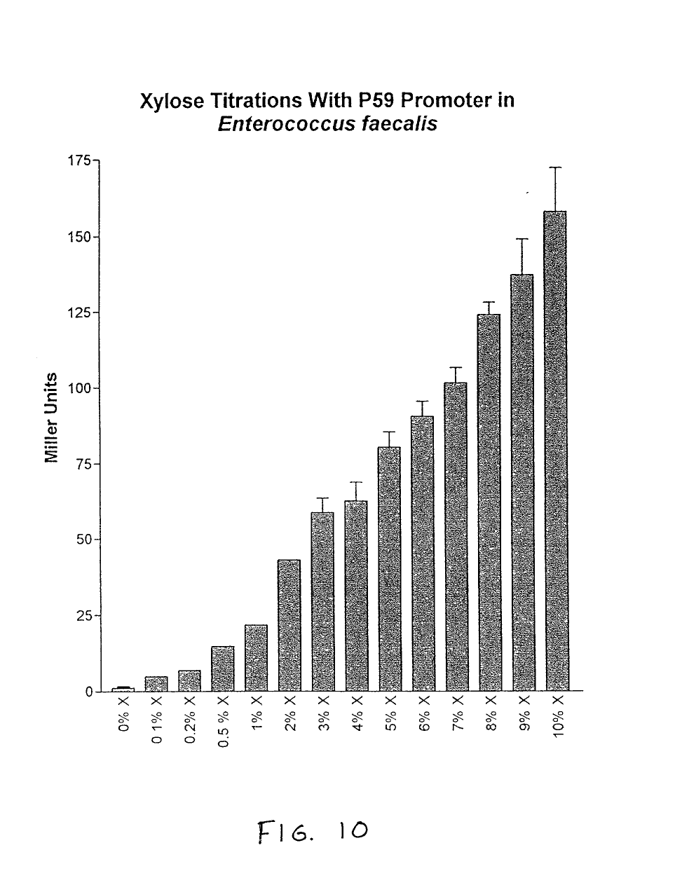

In one example, the derivative of pEPEF1 containing the P59 promoter (pEPEF1-P59) was transformed into Enterococcus faecalis and the transformants were cultured in the presence of increasing concentrations of xylose (from 0.1% to 10%) and in its absense. The β-galactosidase activity produced by the expression of the lacL-lacM reporter genes was measured using the Miller assay as described in Example 5. FIG. 10 shows that the activity of the P59 promoter increases in response to induction with increasing concentrations of xylose.

The next example describes modifications to fusion promoters that effect their transcriptional activity.

### EXAMPLE 7

### Modifications of Fusion Promoters that Alter Transcription Efficiency

This example describes the construction of fusion promoters that have been modified to have altered transcriptional activity in Gram-positive organisms. More specifically, modified fusion promoters, which are derived from wildtype promoters, have been constructed by creating mutations in the promoter sequence which either increase or diminish the strength of the promoter.

In one example, the strong Lactococcus lactis promoter, PL (SEQ ID NO.: 44), was modified to increase its activity in Gram-positive organisms. First, a regulatable PL fusion promoter was constructed by linking the PL promoter to a xylose operator using the methods described previously. Examination of the PL sequence revealed that changing the C at position 45 of SEQ ID NO.: 44 to an A would make the −10 Box region of promoter PL identical to −10 Box consensus site reported for Gram-positive organisms (TATAAT). PL10, a derivative of PL which incorporates this single basepair modification, was constructed by annealing the complementary synthetic oligonucleotides of SEQ ID NOs.: 18 and 19 using the procedure described in Example 1.

| | |
|---|---|
| 5′GACCGTTTCGTGAACTTTTTTGTTGACAAAGATAAAAACACATGATATA | (SEQ ID NO.:18) |
| ATTAAATCAC3′ | |
| 5′TCGAGTGATTTAATTATATCATGTGTTTTTATCTTTGTCAACAAAAAAGTT | (SEQ ID NO.:19) |
| CACGAAACG3′ | |

The underlined nucleotides comprise the 5′-overhang of the RsrII recognition site whereas the italicized nucleotides correspond to the 5′-overhang of the XhoI recognition site. The double stranded promoter formed by the annealing of these two oligonucleotides was then ligated with purified, RsrII/XhoI-digested pEPEF1 to form a pEPEF1 derivative containing the Xly-PL10 fusion promoter. This derivative was termed pEPEF1-PL10. To determine the effect of this modification, the strengths of PL10, PL and P59 were compared by separately transforming pEPEF1 derivatives containing the appropriate promoter into Enterococcus faecalis then culturing the cells in the absense or presence of 5% xylose. The β-galactosidase activity produced by the expression of the lacL-lacM reporter genes was measured using the Miller assay as described in Example 5.

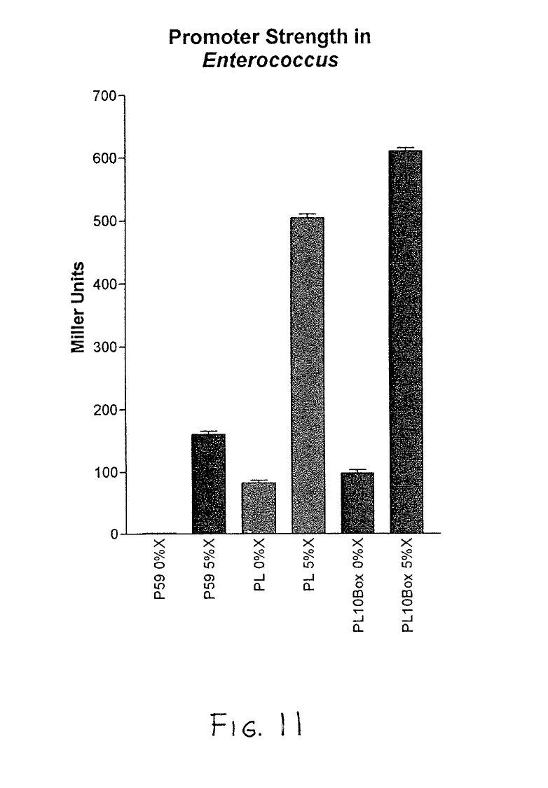

FIG. 11 compares the strengths of the PL, PL10 and P59 fusion promoters expressing the lacL-lacM reporter genes. When induced with 5% xylose, the activity of PL10 is about 20% greater than that of the wildtype PL promoter. Both PL and PL10 have substantially greater activity than P59 under induced conditions. In the absence of xylose, P59 is almost completely repressed whereas both PL and PL10 show significant basal activity.

In another example, P59 was modified to increase its activity in Gram-positive organisms. Examination of the P59 sequence revealed that changing the G at position 37 of SEQ ID NO.: 40 to a T would make the −10 Box region of promoter P59 identical to −10 Box consensus site in Gram-positive organisms. Additionally it appeared that addition of an AT rich region upstream of the −35 Box might enhance promoter activity. A derivative of P59 which incorporates these modifications was constructed by annealing the complementary synthetic oligonucleotides of SEQ ID NOs.: 20 and 21 using the procedure described in Example 1.

| | |
|---|---|
| 5′GACCGAAAAATGACAGTTTATTCTTGACAGGGAGAGATAGGTTTGATAT | (SEQ ID NO.:20) |
| AATATAATAGTTGTC3′ | |
| 5′TCGAGACAACTATTATATTATATCAAACCTATCTCTCCCTGTCAAGAATA | (SEQ ID NO.:21) |
| AACTGTCATTTTTCG3′ | |

The underlined nucleotides comprise the 5′-overhang of the RsrII recognition site whereas the italicized nucleotides correspond to the 5′-overhang of the XhoI recognition site. The double stranded promoter formed by the annealing of these two oligonucleotides was then ligated with purified, RsrII/XhoI-digested pEPEF1, transformed into E. faecalis, and assayed as previously described. When compared to the unmodified P59, the derivative had about 20% higher activity.

It will be appreciated that modified fusion promoters, such as those described above, can be used to construct a regulatable fusion promoter suite which provides a wide dynamic range of both basal and maximal transcriptional activities. Modifying regulatable fusion promoters so as to increase their activity expands the range of both basal and maximal transcriptional activities of a suite of regulatable fusion promoters. Each modified fusion promoter within this suite can be tightly and finely regulated over its own characteristic range of transcriptional activity. Furthermore, the modified regulatable fusion promoters within the suite can comprise different promoters operably linked to the same operators thus providing for a common means of regulation of the promoters within the suite. In some embodiments, the modified promoters within the set of regulatable fusion promoters within the suite can be linked to at least one operator contained in a promoterless expression vector that is capable of replicating in one or more Gram-positive organisms. Accordingly, a regulatable fusion promoter having an appropriate basal level and/or maximal level of transcriptional activity can be selected from the suite for use in the appropriate application.

In yet another example, the Xly-T5 fusion promoter was modified to decrease its activity in Gram-positive organisms. By changing four nucleotides in the −35 Box of SEQ ID NO.: 36 (G to T at postion 18, C to A at position 19, T to C at position 20 and T to A at position 21) and one in the −10 Box of SEQ ID NO.: 36 (A to T at position 43) the Xyl-T5 promoter could be altered to include −10 and −35 elements that are identical to those of the weak xylA promoter. (For a description of the xylA promoter, see Sizemore et al., Mol. Gen. Genet. 227:377-384 (1991) and Schnappinger, D. et al., FEMS Microbiology Letters 129: 121-128 (1995), the disclosures of which are incorporated herein by reference in their entireties). Xyl-T5-DD, a derivative of Xyl-T5 which incorporates these modifications was constructed by annealing the complementary synthetic oligonucleotides of SEQ ID NOs.: 22 and 23 using the procedure described in Example 1.

| | |
|---|---|
| 5′GACCGTCATAAAAAATTTATTTTACATCAGGAAAATTTTTCTGTATATTA | (SEQ ID NO.:22) |
| GATTCAAGTTAGTTTGTTTATTAAATTAACCAACTAAAATGTAG3′ | |
| 5′AATTCTACATTTTAGTTGGTTAATTTAATAAACAAACTAACTTGAATCTA | (SEQ ID NO.:23) |
| ATATACAGAAAAATTTTCCTGATGTAAAATAAATTTTTTATGACG3′ | |

The underlined nucleotides comprise the 5′-overhang of the RsrII recognition site whereas the italicized nucleotides correspond to the 5′-overhang of the EcoRI recognition site. The double stranded promoter formed by the annealing of these two oligonucleotides was then ligated with purified, RsrII/EcrRI-digested pXyl-T5-Reporter (prepared as described in Example 5). The pXyl-T5-Reporter vector was then introduced into S. aureus and the cells were cultured in the presence and absence of 2.0% xylose and expression levels were measured. Xyl-T5-DD had only about 10% of the activity of unmodified Xyl-T5. The nucleotide sequence of pXyl-T5-DD P15a, a Xyl-T5-DD based expression vector, is provided in (SEQ ID NO.: 53).

It will be appreciated that modified fusion promoters, such as those described above, can be used to construct a regulatable fusion promoter suite which provides a wide dynamic range of both basal and maximal transcriptional activities. Modifying regulatable fusion promoters so as to decrease their activity expands the range of both basal and maximal transcriptional activities of a suite of regulatable fusion promoters. Each modified fusion promoter within this suite can be tightly and finely regulated over its own characteristic range of transcriptional activity. Furthermore, the modified regulatable fusion promoters within the suite can comprise different promoters operably linked to the same operators thus providing for a common means of regulation of the promoters within the suite. In some embodiments, the modified promoters within the set of regulatable fusion promoters within the suite can be linked to at least one operator contained in a promoterless expression vector that is capable of replicating in one or more Gram-positive organisms. Accordingly, a regulatable fusion promoter having an appropriate basal level and/or maximal level of transcriptional activity can be selected from the suite for use in the appropriate application.

It will be appreciated that the methods described herein can be used to create promoter sequence modifications that either enhance or reduce promoter activity. These modifications can be made either alone or in combination. Among these changes are modifications of nucleotides within the promoter's −35 and −10 Box elements which make the promoter more or less like the consensus −35 Box and −10 Box sequences (TTGACA and TATAAT, respectively). Other modifications include altering the length of the region between the operator or transcription start site and the −10 Box, altering the length and/or composition of the region between the −10 Box and the −35 Box, and altering the length and/or composition of the AT rich region upstream of the −35 Box (i.e. the −45 Box). It will also be appreciated that modifications which affect promoter activity can include addition of elements such as the CRE region for catabolite repression. These additional elements can be further modified so that they are more or less like their corresponding consensus sequence. Promoters which incorporate one or more modifications similar to those described above are represented by SEQ ID NOs.: 37, 41, 43 and 45.

The experiments described in the Examples 4-7 verify that the fusion promoters disclosed herein can be used to tightly and finely regulate transcription of an RNA or expression of a peptide or a protein throughout a range of activity levels.

It will be appreciated that both modified and unmodified fusion promoters, such as those described above, can be used to construct a regulatable fusion promoter suite which provides a wide dynamic range of both basal and maximal transcriptional activities. Modifying regulatable fusion promoters so as to increase or decrease their activity expands the range of both basal and maximal transcriptional activities of a suite of regulatable fusion promoters. The addition of unmodified fusion promoters having unique basal and/or maximal transcriptional activities also expands the dynamic range of the suite. Each regulatable fusion promoter within this suite can be tightly and finely regulated over its own characteristic range of transcriptional activity. Furthermore, the regulatable fusion promoters within the suite can comprise different promoters operably linked to the same operators thus providing for a common means of regulation of the promoters within the suite. In some embodiments, the promoters within the set of regulatable fusion promoters within the suite can be linked to at least one operator contained in a promoterless expression vector that is capable of replicating in one or more Gram-positive organisms. Accordingly, a regulatable fusion promoter having an appropriate basal level and/or maximal level of transcriptional activity can be selected from the suite for use in the appropriate application. Such a suite of promoters is useful in the methods of identifying genes required for proliferation or methods of identifying compounds which inhibit proliferation described herein.

The examples below discusses methods that use fusion promoters to identify essential genes and compounds having potential antibiotic activity.

### EXAMPLE 8

### Identification of Genes Required for Cellular Proliferation by Expressing Proliferation-Inhibiting Antisense Nucleic Acids from a Fusion Promoter

This example describes the examination of a library of candidate antisense nucleic acid operably linked to fusion promoters to identify essential genes in Staphylococcus aureus and Enterococcus faecalis. Although the following examples utilize the fusion promoters Xyl-T5, CP25 and P59, it will be appreciated that any of the fusion promoters described herein may be used.

It will be appreciated that a regulatable fusion promoter suite which provides a wide dynamic range of both basal and maximal transcriptional activities can be used to facilitate the discovery of proliferation-required genes. Each fusion promoter within this suite can be tightly and finely regulated over its own characteristic range of transcriptional activity. Furthermore, the regulatable fusion promoters within the suite can comprise different promoters operably linked to the same operators thus providing for a common means of regulation of the promoters within the suite. In some embodiments, the promoters within the set of regulatable fusion promoters within the suite can be linked to at least one operator contained in a promoterless expression vector that is capable of replicating in one or more Gram-positive organisms. Accordingly, a regulatable fusion promoter having an appropriate basal level and/or maximal level of transcriptional activity can be selected from the suite based on the expression level of the proliferation-required genes that are targeted for discovery.

In the methods for identifying genes encoding gene products required for cellular proliferation using antisense nucleic acids expressed from an inducible fusion promoter, random genomic fragments are obtained from the organism in which it is desired to identify genes required for cellular proliferation. The random genomic fragments may be generated by a partial digestion with a restriction enzyme, mechanical shearing, using techniques such as sonication and nebulization, or DNAseI digestion. The random genomic fragments are operably linked to a regulatable fusion promoter in a vector, such as one of the expression vectors described herein. In those instances where the inserted genomic fragments are in an antisense orientation with respect to the promoter, the transcript produced is complementary to at least a portion of an mRNA encoding a gene product such that they interact with sense mRNA produced from various genes and thereby decrease the translation efficiency or the level of the sense messenger RNA (mRNA) thus decreasing production of the protein encoded by these sense mRNA molecules. In cases where the sense mRNA encodes a protein required for proliferation, cells grown under inducing conditions fail to grow or grow at a substantially reduced rate. Additionally, in cases where the transcript produced is complementary to at least a portion of a non-translated RNA and where that non-translated RNA is required for proliferation, cells grown under inducing conditions also fail to grow or grow at a substantially reduced rate. In contrast, cells grown under non-inducing conditions grow at a normal rate.

The genes to which the antisense nucleic acids are complementary are then identified and utilized in the methods of the present invention. Thus, to identify genes required for cellular proliferation, the extent of proliferation of cells containing the vectors in the presence of an inducer which induces transcription from the regulatable fusion promoter is compared to the extent of proliferation of cells in the absence of the inducer. Those cells which grow well in the absence of the inducer but exhibit significantly reduced proliferation in the presence of the inducer contain a vector encoding an antisense nucleic acid complementary to at least a portion of a gene required for cellular proliferation.

Use of the above method to identify genes required for cellular proliferation in E. coli, Staphylococcus aureus, Salmonella typhimurium, Klebsiella pneumoniae, Pseudomonas aeruginosa and Enterococcus faecalis has been described in the following U.S. patent applications, the disclosures of which are incorporated herein by reference in their entireties: U.S. patent application Ser. No. 09/815,242, filed Mar. 21, 2001; U.S. patent application Ser. No. 09/492,709, filed Jan. 27, 2000; U.S. patent application Ser. No. 09/711,164, filed Nov. 9, 2000; U.S. patent application Ser. No. 09/741,669, filed Dec. 19, 2000; U.S. patent application Ser. No. 09/815,242, filed Mar. 21, 2001; and U.S. patent application Ser. No. 09/948,993, filed Sep. 6, 2001. The methods used to identify genes required for cellular proliferation in Staphylococcus aureus, and Enterococcus faecalis are summarized below.

Nucleic acids involved in proliferation of Staphylococcus aureus were identified using the EGI technique as follows. A shotgun library of Staphylococcus aureus genomic fragments was cloned into the vector pXyIT5-P15a, which harbors the Xyl-T5 inducible promoter. The vector was linearized at a unique BamHI site immediately downstream of the XyIT5 promoter/operator. The linearized vector was treated with shrimp alkaline phosphatase to prevent reclosure of the linearized ends. Genomic DNA isolated from Staphylococcus aureus strain RN450 was fully digested with the restriction enzyme Sau3A or alternatively, partially digested with DNase I and “blunt-ended” by incubating with T4 DNA polymerase. Random genomic fragments between 200 and 800 base pairs in length were selected by gel purification. The size-selected genomic fragments were added to the linearized and dephosphorylated vector at a molar ratio of 0.1 to 1, and ligated to form a shotgun library.

The ligated products were transformed into electrocompetent E. coli strain XL1-Blue MRF (Stratagene) and plated on LB medium with carbenicillin at 100 μg/ml. Resulting colonies numbering 5×10⁵ or greater were scraped and combined, and were then subjected to plasmid purification.

The purified library was then transformed into electrocompetent Staphylococcus aureus strain RN4220. Resulting transformants were plated on LBG agar with chloramphenicol at 15 μg/ml in order to generate 100 to 150 platings at 500 colonies per plating. The colonies were subjected to robotic picking and arrayed into wells of 384 well culture dishes. Each well contained 100 μl of LBG+CM15 liquid medium. Inoculated 384 well dishes were incubated 16 hours at 37° C., and each well was robotically gridded onto solid LBG+CM15 medium with or without 2% xylose. Gridded plates were incubated 16 hours at 37° C., and then manually scored for arrayed colonies that were growth-compromised in the presence of xylose. Such growth sensitivity occurred at a frequency of one in sixty arrayed colonies.

Arrayed colonies that were growth-sensitive on medium containing 2% xylose, yet were able to grow on similar medium lacking xylose, were subjected to further growth sensitivity analysis as follows: Colonies from the plate lacking xylose were manually picked and inoculated into individual wells of a 96 well culture dish containing LBG+CM15, and were incubated for 16 hours at 37° C. These cultures were robotically diluted 1/100 into fresh medium and allowed to incubate for 4 hours at 37° C., after which they were subjected to serial dilutions in a 384 well array and then gridded onto media with and without 2% xylose. After growth for 16 hours at 37° C., the arrays of serially diluted spots that resulted were compared between the two media. Spots that grew similarly on both media were scored as a negative and corresponding colonies were no longer considered. Spots on xylose medium that failed to grow to the same serial dilution compared to those on the non-xylose plate were given a score based on the differential, i.e. should the spots on xylose only grow to a serial dilution of −4 while they were able to grow to −8 on the non-xylose plate, then the corresponding transformant colony received a score of “4” representing the log difference in growth observed.

Nucleic acids involved in proliferation of E. faecalis were identified as follows. A shotgun library of E. faecalis genomic fragments was cloned into either pEPEF3 or pEPEF14, which contain the CP25 or P59 promoter, respectively, regulated by the xyl operator/repressor. The vector was linearized at a unique SmaI site immediately downstream of the promoter/operator. The linearized vector was treated with alkaline phosphatase to prevent reclosure of the linearized ends. Genomic DNA isolated from E. faecalis strain OG1RF was partially digested with DNase I and “blunt-ended” by incubating with T4 DNA polymerase. Random genomic fragments between 200 and 800 base pairs in length were selected by gel purification. The size-selected genomic fragments were added to the linearized and dephosphorylated vector at a molar ratio of 2 to 1, and ligated to form a shotgun library.

The ligated products were transformed into electrocompetent E. coli strain TOP10 cells (Invitrogen) and plated on LB medium with erythromycin (Erm) at 150 μg/mL. Resulting colonies numbering 5×10⁵ or greater were scraped and combined, and were then subjected to plasmid purification.

The purified library was then transformed into electrocompetent E. faecalis strain OG1RF. Resulting transformants were plated on Todd-Hewitt (TH) agar with erythromycin at 10 μg/mL in order to generate 100 to 150 platings at 500 colonies per plating. The colonies were subjected to robotic picking and arrayed into wells of 384 well culture dishes. Each well contained 100 μL of THB+Erm 10 μg/mL. Inoculated 384 well dishes were incubated 16 hours at room temperature, and each well was robotically gridded onto solid TH agar+Erm with or without 5% xylose. Gridded plates were incubated 16 hours at 37° C., and then manually scored for arrayed colonies that were growth-compromised in the presence of xylose.

Arrayed colonies that were growth-sensitive on medium containing 5% xylose, yet were able to grow on similar medium lacking xylose, were subjected to further growth sensitivity analysis. Colonies from the plate lacking xylose were manually picked and inoculated into individual wells of a 96 well culture dish containing THB+Erm 10, and were incubated for 16 hours at 30° C. These cultures were robotically diluted 1/100 into fresh medium and allowed to incubate for 4 hours at 37° C., after which they were subjected to serial dilution on plates containing 5% xylose or plates lacking xylose. After growth for 16 hours at 37° C., the arrays of serially diluted spots that resulted were compared between the two media. Colonies that grew similarly on both media were scored as a negative and corresponding colonies were no longer considered. Colonies on xylose medium that failed to grow to the same serial dilution compared to those on the non-xylose plate were given a score based on the differential. For example, colonies on xylose medium that only grow to a serial dilution of −4 while they were able to grow to −8 on the non-xylose plate, then the corresponding transformant colony received a score of “4” representing the log difference in growth observed.

Of the 1.4 million Enterococcus faecalis colonies screened using the CP25 fusion promoter, about 1 of every 3750 colonies had reduced proliferation ability whereas of the 390,000 colonies screened using the P59 fusion promoter, about 1 of every 390 colonies had reduced proliferation ability.

Thus, it will be appreciated that use of a regulatable fusion promoter from the suite of regulatable fusion promoters which has a higher level of transcriptional activity may increase the frequency at which proliferation-required genes are identified and may allow identification of proliferation-required genes which would not be identified using weaker promoters. Such a fusion promoter would be expected to produce a larger number of colonies having reduced proliferation ability per colonies screened. Furthermore, proliferation-required genes from new gene classes would be expected.

Expression vectors were purified from Staphylococcus aureus or Enterococcus faecalis colonies identified as having reduced proliferation upon induction of the fusion promoter. The nucleic acids contained on the expression vectors under control of the fusion promoter were isolated for subsequent nucleotide sequence determination and further characterization.

It will be appreciated that the random genomic fragments used in the above methods can be generated using restriction enzymes and endonucleases other than those specifically described or by any other procedure. In addition, random genomic fragments may be generated by mechanical shearing. Sonication and nebulization are two such techniques commonly used for mechanical shearing of DNA.

The next example describes experiments that use the Rapid Replacement of Genomic Promoters technique to identify essential genes for use as targets for drug discovery.

### EXAMPLE 9

### Replacement of Genomic Promoters for Proliferation-Required Genes with Inducible Fusion Promoters

Nucleic acids involved in proliferation of Staphylococcus aureus or Enterococcus faecalis are identified using the Rapid Replacement of Genomic Promoters technique described in U.S. patent application Ser. No. 09/948,993, filed Sep. 6, 2001, the disclosure of which is incorporated herein by reference in its entirety, and U.S. Provisional Patent Application Serial No. 60/230347, filed Sep. 6, 2000, the disclosure of which is incorporated herein by reference in its entirety. Initially, a target gene is selected. The gene may be a gene which is known or suspected to be essential for proliferation or a gene which has not been characterized with respect to its essentiality for proliferation. Identification of the essential gene can be accomplished by using the EGI technique or other methods including, but not limited to, genes shown to be essential in the literature or genes of unknown essentiality that are predicted to be essential by bioinformatics. Additionally, one can target essential operons containing multiple genes.

Next, the native promoter of the target gene or operon is replaced with a regulatable promoter, such as the fusion promoters described herein using “ET cloning” technology. Briefly, this is accomplished by engineering the one of the fusion promoters described herein such that it is 3′ of a selectable marker (e.g., a CAT gene). This entire cassette is amplified by PCR. This PCR product becomes the template for another round of PCR using primers with 80 bp of homology to the target promoter and 20 bp of homology to the fusion promoter-containing cassette. However, as little as 60 bp homology to the target promoter can be used. The region of homology is chosen such that the cassette will replace the promoter of the target gene without effecting the endogenous Shine-Delgamo sequence. This construct is then transformed into competent cells containing various levels of inducer (e.g. xylose or analog thereof) to assure that the correct level of expression is achieved to allow survival. In some embodiments, the native promoter in a strain having an enhanced frequency of homologous recombination may be replaced with a regulatable promoter.

The correct integration of the cassette can be confirmed by colony PCR as described in U.S. patent application Ser. No. 09/948,993, filed Sep. 6, 2001, and U.S. Provisional Patent Application entitled Rapid PCR Method for Determination of Whether a Gene is Essential, Serial No. 60/230347, filed Sep. 6, 2000, attorney docket number ELITRA.022PR, the disclosures of which are incorporated herein by reference in their entireties. Preferably, correct integrants are tested for growth defects as inducer is titrated away. The inability to grow in the absence of inducer will confirm that the target was essential. These strains can then be induced to a level determined to be the optimal balance between slowed growth and sensitivity to inhibition of the target protein for use in a cell-based assay for compounds with antibiotic activity or may be used to identify genes required for proliferation as described herein.

The next example describes experiments that use an operator insertion technique to identify essential genes for use as targets for drug discovery.

### EXAMPLE 10

### Regulation of Genomic Promoters for Proliferation-Required Genes by Operator Insertion

In this example, insertion of an operator into the promoter of a proliferation-required gene is described. A target promoter which drives the expression of a target gene or operon is identified. The target gene may be a gene which is known to be required for proliferation, suspected to be required for proliferation, or a gene which has not been characterized with respect to whether it is required for proliferation. An oligonucleotide comprising a xyl, tet, trp, mal, λc1 or lac operator flanked on each side by 40 nucleotides homologous to the target promoter is synthesized. The 40 nucleotide flanking sequences are determined based on the desired location for intergration of the xyl operator into the promoter. The single stranded oligonucleotide construct is then transformed into a bacterium having an enhanced frequency of homologous recombination. For example, the bacterium may express the λ Beta and Gam proteins. The cells in the transformation mixture are diluted and plated on medium containing xylose. Colonies in which the operator has integrated into the target promoter are identified by colony PCR. The identified colonies are grown in medium containing or lacking inducer. The colonies proliferate on medium containing inducer but fail to grow on medium lacking inducer, thereby indicating the target promoter directs transcription of a gene encoding a gene product required for proliferation. Strains in which a gene encoding a gene product required for proliferation is under the control of the xyl, tet, trp, mal, λc1 or lac operator may also be used to identify compounds which inhibit proliferation in the cell-based asays described herein.

It will be appreciated that operators other than the xyl, tet, trp, mal, λc1 or lac operator can be used with the methods described above.

The next example describes experiments that use the Random Insertional Cis-Antisense technique to identify essential genes for use as targets for drug discovery.

### EXAMPLE 11

### Insertional Inactivation of Proliferation-Required Genes by Random Insertion of an Inducible Fusion Promoter

Nucleic acids involved in proliferation of Staphylococcus or Enterococcus are identified using the Random Insertional Cis-Antisense technique described in U.S. Provisional Patent Application Serial No. 60/230,403, filed Sep. 6, 2000, the disclosure of which is incorporated herein by reference in its entirety. In one embodiment, a vector containing an outward-directed inducible fusion promoter is randomly integrated into the genome of a Gram-positive organism such as S. aureus or E. faecalis or the other Gram-positive organisms described herein and clones containing promoters in cis-antisense orientation with regard to essential genes are identified. Accordingly, a vector containing an inducible promoter, such as one of the fusion promoters described herein, is first engineered such that the inducible promoter reads into a multiple cloning site (MCS). The genetic marker is juxtaposed directly 3′ of the MCS. This positioning helps ensure that the transforming vector will not be degraded by exonucleases (transformants in which the vector is degraded will not be recovered because the genetic marker will be lost). The vector is linearized by cutting with any single restriction enzyme in the MCS. Using different enzymes to linearize may increase the randomness of integration of the vector. The linearized vector is then transformed into the appropriate bacterial strain.

The intergrative transformation frequency of linear DNA varies widely among bacterial strains. In some strains, linear vectors usually transform integratively at a significantly higher frequency than circular vectors. This is especially true in bacterial strains that have been modified to have an enhanced frequency of homologous recombination. In strains in which the frequency of integrative transformation of linear vectors is low, circular vectors often transform integratively at a higher frequency. The mechanism of integration is largely unknown but it is contemplated that integration occurs via nonhomologous recombination. Sites of integration appear to be entirely random. Transformants are robotically picked, grown, and are robotically gridded onto inducing and noninducing (inducing or not inducing transcription of the promoter) media. These gridded plates are then screened for transformants that have a pronounced growth defect on inducing media relative to the phenotype on noninducing media. Such transformants may have the vector integrated into their genome in several possible locations in an essential gene: the 3′ untranslated region or any part of the coding or noncoding region that does not result in disruption of function of the gene product. In most cases the inducible promoter is oriented such that it produces an antisense RNA from the target gene. Thus, when the promoter is induced the expression of the essential gene is reduced. Such inhibition can occur by one of several mechanisms including binding of the antisense RNA to the mRNA, colliding RNA polymerases, RNA interference (RNAi) or a combination of such phenomenon.

The identity of the essential gene is then determined by sequencing the junction of the vector with the essential gene, the junction where the inducible promoter is juxtaposed with genomic DNA. This assay can also be used to create bacterial strains for cell-based assays that evaluate candidate antibiotic compounds as described herein.

The next example describes approaches that were used to determine the identity of the newly discovered essential genes.

### EXAMPLE 12

### Nucleotide Sequence Determination of Genes that are Affected by Proliferation-Inhibiting Antisense Nucleic Acids Expressed from Fusion Promoters

With reference to experiments that were performed using the antisense technique, this example describes approaches that were used to determine the nucleic acid sequence of newly discovered essential genes. It should be understood that the teachings herein can also be used to determine the nucleic acid sequence of essential genes identified using the promoter replacement, operator insertion or Random Insertional Cis-Antisense technique.

Plasmids from transformant colonies that received a dilution plating score of “2” or greater were isolated to obtain the genomic DNA insert responsible for growth inhibition as follows. Staphylococcus aureus were grown in standard laboratory media (LB or TB with 15 ug/ml Chloramphenicol to select for the plasmid). Growth was carried out at 37° C. overnight in culture tubes or 2 ml deep well microtiter plates. Lysis was performed as follows. Cultures (2-5 ml) were centrifuged and the cell pellets resuspended in 1.5 mg/ml solution of lysostaphin (20 μl/ml of original culture) followed by addition of 250 μl of resuspension buffer (Qiagen). Alternatively, cell pellets were resuspended directly in 250 μl of resuspension buffer (Qiagen) to which 5-20 μl of a 1 mg/ml lysostaphin solution were added. DNA was isolated using Qiagen miniprep kits or Wizard (Qiagen) miniprep kits according to the instructions provided by the manufacturer.

The genomic DNA inserts were then amplified from the purified plasmids by PCR as follows. Approximately, 1:1 of Qiagen purified plasmid was put into a total reaction volume of 25:1 Qiagen Hot Start PCR mix. The following primers were used in the PCR reaction:

| | | |
|---|---|---|
| pXylT5F: | CAGCAGTCTGAGTTATAAAATAG | (SEQ ID NO:24) |
| LexL | TGTTTTATCAGACCGCTT | (SEQ ID NO:25) |

PCR was carried out in a PE GenAmp with the following cycle times:

Step 1. 95° C. 15 min

Step 2. 94° C. 45 sec

Step 3. 54° C. 45 sec

Step 4. 72° C. 1 minute

Step 5. Return to step 2 (29 times)

Step 6. 72° C. 10 minutes

Step 7. 4° C. hold

The PCR products were cleaned using Qiagen Qiaquick PCR plates according to the manufacturer's instructions. The amplified genomic DNA inserts were then subjected to automated sequencing.

For E. faecalis, plasmids from transformant colonies that received a dilution plating score of “2” or greater were isolated to obtain the genomic DNA insert responsible for growth inhibition as follows. E. faecalis were grown in THB 10 μg/ml Erm at 30° C. overnight in 100 ul culture wells in microtiter plates. To amplify insert DNA 2 ul of culture were placed into 25 μl Qiagen Hot Start PCR mix. PCR reactions were in 96 well microtiter plates. Primers to vector regions flanking the insert were used in the PCR reaction. PCR was carried out in a PE GenAmp with the following cycle times:

Step 1. 95° C. 15 min

Step 2. 94° C. 45 sec

Step 3. 54° C. 45 sec

Step 4. 72° C. 1 minute

Step 5. Return to step 2, 29 times

Step 6. 72° C. 10 minutes

Step 7. 4° C. hold

The PCR products were cleaned using Qiagen Qiaquick PCR plates according to the manufacturer's instructions. The purified PCR products were then directly cycle sequenced with Qiagen Hot Start PCR mix. PCR was carried out in a PE GenAmp with the following cycle times:

Step 1. 94° C. 15 min

Step 2. 96° C. 10 sec

Step 3. 50° C. 5 sec

Step 4. 60° C. 4 min

Step 5. Return to step 2, 24 times

Step 6. 4° C. hold

The PCR products were cleaned using Qiagen Qiaquick PCR plates according to the manufacturer's instructions. The amplified genomic DNA inserts were then subjected to automated sequencing.

The example below describes techniques that were used to compare the sequences identified by the approaches above to sequences present in publicly available databases so as to better characterize the essential genes.

### EXAMPLE 13

### Comparison of Isolated Nucleic Acids to Known Sequences

With reference to experiments that were performed using the antisense technique, this example describes approaches that were used to characterize the nucleic acid sequence of newly discovered essential genes in S. aureus and E. faecalis. It should be understood that the teachings herein can also be used to determine the nucleic acid sequence of essential genes in other Gram-positive cells and essential genes identified in Gram-positive cells using the promoter replacement, operator insertion or Random Insertional Cis-Antisense technique.

The nucleic acid sequences of the subcloned fragments from S. aureus or E. faecalis obtained from the expression vectors containing the Xyl-T5 and CP25 fusion promoters discussed above were compared to known sequences from S. aureus or E. faecalis and other microorganisms as follows. First, to confirm that each clone originated from one location on the chromosome and was not chimeric, the sequences of the selected clones were compared against the S. aureus or E. faecalis genomic sequences to align the clone to the correct position on the chromosome. The NCBI BLASTN v 2.0.9 program was used for this comparison, and the incomplete Staphylococcus aureus genomic sequences licensed from TIGR, as well as the NCBI nonredundant GenBank database were used as the source of genomic data. The E. faecalis sequences were compared to a proprietary database.

The BLASTN analysis was performed using the default parameters except that the filtering was turned off. No further analysis was performed on inserts which resulted from the ligation of multiple fragments.

In general, antisense molecules and their complementary genes are identified as follows. First, all possible full length open reading frames (ORFs) are extracted from available genomic databases. Such databases include the GenBank nonredundant (nr) database, the unfinished genome database available from TIGR and the PathoSeq database developed by Incyte Genomics. The latter database comprises over 40 annotated bacterial genomes including complete ORF analysis. If databases are incomplete with regard to the bacterial genome of interest, it is not necessary to extract all ORFs in the genome but only to extract the ORFs within the portions of the available genomic sequences which are complementary to the clones of interest. Computer algorithms for identifying ORFs, such as GeneMark, are available and well known to those in the art. Comparison of the clone DNA to the complementary ORF(s) allows determination of whether the clone is a sense or antisense clone. Furthermore, each ORF extracted from the database can be compared to sequences in well annotated databases including the GenBank (nr) protein database, SWISSPROT and the like. A description of the gene or of a closely related gene in a closely related microorganism is often available in these databases. Similar methods are used to identify antisense clones corresponding to genes encoding non-translated RNAs.

In some cases, for example, the NCBI BLASTN 2.0.9 computer algorithm was used and the default parameters were used with the exception that filtering was turned off. The default parameters for the BLASTN and BLASTX analyses were:

Expectation value (e)=10

Alignment view options: pairwise

Filter query sequence (DUST with BLASTN, SEG with others)=T

Cost to open a gap (zero invokes behavior)=0

Cost to extend a gap (zero invokes behavior)=0

X dropoff value for gapped alignment (in bits) (zero invokes behavior)=0

Show GI's in deflines=F

Penalty for a nucleotide mismatch (BLASTN only)=!3

Reward for a nucleotide match (BLASTN only)=1

Number of one-line descriptions (V)=500

Number of alignments to show (B)=250

Threshold for extending hits=default

Perform gapped alignment (not available with BLASTX)=T

Query Genetic code to use=1

DB Genetic code (for TBLAST[nx] only=1

Number of processors to use=1

SeqAlign file

Believe the query defline=F

Matrix=BLOSUM62

Word Size=default

Effective length of the database (use zero for the real size)=0

Number of best hits from a region to keep=100

Length of region used to judge hits=20

Effective length of the search space (use zero for the real size)=0

Query strands to search against database (for BLAST[nx] and TBLASTX), 3 is both, 1 is top, 2 is bottom=3

Produce HTML output=F

Those ORFs found to be complementary to the cloned nucleic acid sequences discussed above were evaluated with regard to orientation. Antisense clones were identified as those clones for which transcription from the inducible promoter would result in the expression of an RNA antisense to the complementary ORF.

The gene descriptions in the PathoSeq database derive from annotations available in the public sequence databases described above. It will be appreciated that ORFs may also be identified using databases other than PathoSeq. For example, the ORFs may be identified using the methods described in U.S. Provisional Patent Application Serial No. 60/191,078, filed Mar. 21, 2000, the disclosure of which is incorporated herein by reference in its entirety.

In the antisense-based screening experiments in Enterococcus faecalis as described in Examples 8 and 9, 256 proliferation-inhibiting antisense sequences corresponding to 61 unique ORFs were discovered using the CP25 fusion promoter whereas 790 proliferation-inhibiting antisense sequences corresponding to 150 unique ORFs were discovered using the P59 fusion promoter.

The example below describes approaches that may be used to identify operons or downstream genes associated with the essential genes identified by the methods above.

### EXAMPLE 14

### Identification of Genes and their Corresponding Operons that are Affected by Proliferation-Inhibiting Antisense Nucleic Acids Expressed from Fusion Promoters

With reference to experiments that were performed using the antisense technique, this example describes approaches that may be used to identify operons or downstream genes associated with the newly discovered essential genes in S. aureus and E. faecalis. It should be understood that the teachings herein can also be used to identify operons or downstream genes associated with the essential genes in other Gram-positive cells and essential genes identified in Gram-positive cells using the promoter replacement, operator insertion or Random Insertional Cis-Antisense technique Once the genes involved in S. aureus and E. faecalis proliferation are identified as described above, the operons in which these genes lie may be identified by comparison with known microbial genomes. Since bacterial genes are expressed in a polycistronic manner, the antisense inhibition of a single gene in an operon might affect the expression of all the other genes on the operon or the genes downstream from the single gene identified. Accordingly, each of the genes contained within an operon may be analyzed for their effect on proliferation.

Operons are predicted by looking for all adjacent genes in a genomic region that lie in the same orientation with no large noncoding gaps in between. First, full length ORFs complementary to the antisense molecules are identified as described above. Adjacent ORFs are then identified and their relative orientation determined either by directly analyzing the genomic sequences surrounding the ORFs complementary to the antisense clones or by extracting adjacent ORFs from the collection obtained through whole genome ORF analysis described above followed by ORF alignment. Operons predicted in this way may be confirmed by comparison to the arrangement of the homologous genes in the Bacillus subtilis complete genome sequence, as reported by the genome database compiled at Institut Pasteur Subtilist Release R15.1 (Jun. 24, 1999) which can be found at http://bioweb.pasteur.fr/GenoList/SubtiList/. The Bacillus subtilis genome is the only fully sequenced and annotated genome from a Gram-positive microorganism, and appears to have a high level of similarity to Staphylococcus aureus both at the level of conservation of gene sequence and genomic organization including operon structure. Annotation of some of the DNA sequences in some of the aforementioned databases is lacking, but comparisons may be made to E. coli using tools such as BLASTX. Public or proprietary databases may be used to analyzed E. faecalis sequences as well as the databases listed above.

Once the full length ORFs and/or the operons containing them have been identified using the methods described above, they can be obtained from a genomic library by performing a PCR amplification using primers at each end of the desired sequence. Those skilled in the art will appreciate that a comparison of the ORFs to homologous sequences in other microorganisms will facilitate confirmation of the start and stop codons at the ends of the ORFs.

In some embodiments, the primers may contain restriction sites which facilitate the insertion of the gene or operon into a desired vector. For example, the gene may be inserted into an expression vector and used to express the proliferation-required protein as described below. Other methods for obtaining the full length ORFs and/or operons are familiar to those skilled in the art. For example, natural restriction sites may be employed to insert the full length ORFs and/or operons into a desired vector.

The next example describes approaches that may be used to identify individual genes within an operon that was characterized as being required for proliferation.

### EXAMPLE 15

### Identification of Individual Genes within an Operon Required for Proliferation

With reference to experiments that were performed using the antisense technique, this example describes approaches that may be used to identify individual genes within an operon that has been characterized as being required for proliferation of a Gram-positive organism such as S. aureus or E. faecalis or the other Gram-positive organisms listed herein. It should be understood that the teachings herein can also be used to identify individual genes within an operon that were characterized as being required for proliferation in other Gram-positive cells and can be used in conjunction with the promoter replacement, operator insertion or Random Insertional Cis-Antisense technique.

The strategy described in this example seeks to determine if a targeted gene within an operon is required for cell proliferation by replacing the targeted gene in the chromosome with an in-frame deletion of the coding region of the targeted gene. Deletion inactivation of a chromosomal copy of a gene in Staphylococcus aureus or Enterococcus faecalis can be accomplished by integrative gene replacement. The principles of this method were described in Xia, M., et al. 1999 Plasmid 42:144-149 and Hamilton, C. M., et al 1989. J. Bacteriol. 171: 4617-4622, the disclosures of which are incorporated herein by reference in their entireties. In this approach, a mutant allele of the targeted gene is constructed by way of an in-frame deletion and introduced into the chromosome using a suicide vector. This results in a tandem duplication comprising a deleted (null) allele and a wild type allele of the target gene. Cells in which the vector sequences have been deleted are isolated using a counter-selection technique. Removal of the vector sequence from the chromosomal insertion results in either restoration of the wild-type target sequence or replacement of the wild type sequence with the deletion (null) allele. E. faecalis genes can be disrupted using a suicide vector that contains an internal fragment to a gene of interest. With the appropriate selection this plasmid will homologously recombine into the chromosome (Nallapareddy, S. R., X. Qin, G. M. Weinstock, M. Hook, B. E. Murray. 2000. Infect. Immun. 68:5218-5224, the disclosure of which is incorporated herein by reference).

The resultant population of Staphylococcus aureus, Enterococcus faecalis, or other Gram-positive colonies can then be evaluated to determine whether the target sequence is required for proliferation by PCR amplification of the affected target sequence. If the targeted gene is not required for proliferation, then PCR analysis will show that roughly equal numbers of colonies have retained either the wild-type or the mutant allele. If the targeted gene is required for proliferation, then only wild-type alleles will be recovered in the PCR analysis.

The method of cross-over PCR is used to generate the mutant allele by amplification of sequences flanking but not including the coding region of the gene of interest, such that overlap between the resulting two PCR amplification products allows them to hybridize. Further PCR amplification of this hybridization product using primers representing the extreme 5′ and 3′ ends can produce an amplification product containing an in-frame deletion of the coding region but retaining substantial flanking sequences.

For Staphylococcus aureus, this amplification product is subcloned into the suicide vector pSA3182 (Xia, M., et al. 1999 Plasmid 42:144-149, the disclosure of which is incorporated herein by reference in its entirety) which is host-dependent for autonomous replication. This vector includes a tetC tetracycline-resistance marker and the origin of replication of the well-known Staphylococcus aureus plasmid pT181 (Mojumdar, M and Kahn, S. A., Characterisation of the Tetracycline Resistance Gene of Plasmid pT181, J. Bacteriol. 170: 5522 (1988), the disclosure of which is incorporated herein by reference in its entirety). The vector lacks the repC gene which is required for autonomous replication of the vector at the pT181 origin. However autonomous replication of this vector can occur in a Staphylococcus aureus host strain such as SA3528, which expresses repC in trans. Once the amplified truncated target gene sequence is cloned and propagated in the pSA3182 vector, it can then be introduced into a repC minus strain such as RN4220 (Kreiswirth, B. N. et al., The Toxic Shock Syndrome Exotoxin Structural Gene is Not Detectably Transmitted by a Prophage, Nature 305:709-712 (1983), the disclosure of which is incorporated herein by reference in its entirety) by electroporation with selection for tetracycline resistance. In this strain, the vector must integrate by homologous recombination at the targeted gene in the chromosome to impart drug resistance. This results in a inserted truncated copy of the allele, followed by pSA3182 vector sequence, and finally an intact and functional allele of the targeted gene.

Once a tetR Staphylococcus aureus strain is isolated from the above technique and shown to include truncated and wild-type alleles of the targeted gene as described above, a second plasmid, pSA7592 (Xia, M., et al. 1999 Plasmid 42:144-149, the disclosure of which is incorporated herein by reference in its entirety) is introduced into the strain by electroporation. This gene includes an erythromycin resistance gene and a repC gene that is expressed at high levels. Expression of repC in these transformants is toxic due to interference of normal chromosomal replication at the integrated pT181 origin of replication. This counterselects for strains that have removed the vector sequence by homologous recombination, resulting in either of two outcomes: The counterselected cells either possess a wild-type allele of the targeted gene or a gene in which the wild-type allele has been replaced by the engineered in-frame deletion of the truncated allele.

PCR amplification can be used to test either outcome among the resulting erythromycin resistant, tet sensitive transformant colonies. If the targeted gene is not required for cellular replication, then PCR evidence for both wild-type and mutant alleles will be found among the population of resultant transformants. However, if the targeted gene is required for cellular replication, then only the wild-type form of the gene will be evident among the resulting transformants.

Similarly, for Enterococcus faecalis the PCR products containing the mutant allele of the target sequence may be introduced into an appropriate knockout vector and cells in which the wild type target has been disrupted are selected using the appropriate methodology.

The above methods have the advantage that insertion of an in-frame deletion mutation is far less likely to cause downstream polar effects on genes in the same operon as the targeted gene. However, it will be appreciated that other methods for disrupting Staphylococcus aureus or Enterococcus faecalis, which are familiar to those skilled in the art may also be used. Each gene in the operon may be disrupted using the methodology above to determine whether it is required for proliferation.

The example below describes approaches to express genes involved in cell proliferation.

### EXAMPLE 16

### Expression of the Proteins Encoded by Genes Identified as Required for Staphylococcus aureus or Enterococcus faecalis Proliferation

The following is provided as one exemplary method to express the proliferation-required proteins encoded by sequences identified by the methods described herein using expression systems designed either for E. coli, Staphylococcus aureus, or Enterococcus faecalis. First, the initiation and termination codons for the gene are identified. If desired, methods for improving translation or expression of the protein are well known in the art. For example, if the nucleic acid encoding the polypeptide to be expressed lacks a methionine codon to serve as the initiation site, a strong Shine-Dalgarno sequence, or a stop codon, these sequences can be added. Similarly, if the identified nucleic acid sequence lacks a transcription termination signal, this sequence can be added to the construct by, for example, splicing out such a sequence from an appropriate donor sequence. In addition, the coding sequence may be operably linked to a strong promoter or an inducible promoter, such as the fusion promoters described herein, if desired. The identified nucleic acid sequence or portion thereof encoding the polypeptide to be expressed is obtained by, for example, PCR from the bacterial expression vector or genome using oligonucleotide primers complementary to the identified nucleic acid sequence or portion thereof and containing restriction endonuclease sequences appropriate for inserting the coding sequences into the vector such that the coding sequences can be expressed from the vector's promoter. Alternatively, other conventional cloning techniques may be used to place the coding sequence under the control of the promoter. In some embodiments, a termination signal may be located downstream of the coding sequence such that transcription of the coding sequence ends at an appropriate position.

Several expression vector systems for protein expression in E. coli are well known and available to someone knowledgeable in the art. The coding sequence may be inserted into any of these vectors and placed under the control of the promoter. The expression vector may then be transformed into DH5α or some other E. coli strain suitable for the over expression of proteins.

Alternatively, an expression vector encoding a protein required for proliferation of Staphylococcus aureus or Enterococcus faecalis may be introduced into Staphylococcus aureus or Enterococcus faecalis. In some embodiments, gene encoding the protein required for proliferation may be operably linked to one of the fusion promoters described herein. Electroporation protocols for introducing nucleic acids into Staphylococcus aureus and Enterococcus faecalis are well known in the art. For example, the electroporation protocol described in Staphylococcus aureus transformation protocols described in J. C. Lee “Electroporation of Staphylococci” from Methods in Molecular Biology vol 47: Electroporation Protocols for Microorganisms Edited by: J. A. Nickoloff Humana Press Inc., Totowa, N.J. pp209-216, the disclosure of which is incorporated herein by reference in its entirety, may be used. Positive transformants are selected after growing the transformed cells on plates containing an antibiotic to which the vector confers resistance. For example, Staphylococcus aureus may be transformed with an expression vector in which the coding sequence is operably linked to the fusion promoters described herein, such as fusion promoters comprising the T5 promoter operably linked to a xyl operator such that expression of the encoded protein is inducible with xylose. Such an example vector is pXyl-T5 p15a. In another example, Enterococcus faecalis may be transformed with an expression vector in which the coding sequence is operably linked to the fusion promoters described herein, such as fusion promoters comprising the PL promoter operably linked to a xyl operator such that expression of the encoded protein is inducible with xylose. Such an example vector is pEPEF21.

In one embodiment, the protein is expressed and maintained in the cytoplasm as the native sequence. In an alternate embodiment, the expressed protein can be modified to include a protein tag that allows for differential cellular targeting, such as to the periplasmic space of Gram-negative or Gram-positive expression hosts or to the exterior of the cell (i.e., into the culture medium). In some embodiments, the osmotic shock cell lysis method described in Chapter 16 of Current Protocols in Molecular Biology, Vol. 2, (Ausubel, et al., Eds.) John Wiley & Sons, Inc. (1997) may be used to liberate the polypeptide from the cell. In still another embodiment, such a protein tag could also facilitate purification of the protein from either fractionated cells or from the culture medium by affinity chromatography. Each of these procedures can be used to express a proliferation-required protein.

Expressed proteins, whether in the culture medium or liberated from the periplasmic space or the cytoplasm, are then purified or enriched from the supernatant using conventional techniques such as ammonium sulfate precipitation, standard chromatography, immunoprecipitation, immunochromatography, size exclusion chromatography, ion exchange chromatography, and HPLC. Alternatively, the polypeptide may be secreted from the host cell in a sufficiently enriched or pure state in the supernatant or growth media of the host cell to permit it to be used for its intended purpose without further enrichment. The purity of the protein product obtained can be assessed using techniques such as SDS PAGE, which is a protein resolving technique well known to those skilled in the art. Coomassie, silver staining or staining with an antibody are typical methods used to visualize the protein of interest.

Antibodies capable of specifically recognizing the protein of interest can be generated using synthetic peptides using methods well known in the art. See, Antibodies: A Laboratory Manual, (Harlow and Lane, Eds.) Cold Spring Harbor Laboratory (1988). For example, 15-mer peptides having a sequence encoded by the appropriate identified gene sequence of interest or portion thereof can be chemically synthesized. The synthetic peptides are injected into mice to generate antibodies to the polypeptide encoded by the identified nucleic acid sequence of interest or portion thereof. Alternatively, samples of the protein expressed from the expression vectors discussed above can be purified and subjected to amino acid sequencing analysis to confirm the identity of the recombinantly expressed protein and subsequently used to raise antibodies.

The protein encoded by the identified nucleic acid sequence of interest or portion thereof can be purified using standard immunochromatography techniques. In such procedures, a solution containing the desired protein, such as the culture medium or a cell extract, is applied to a column having antibodies against the secreted protein attached to the chromatography matrix. The desired protein is allowed to bind the immunochromatography column. Thereafter, the column is washed to remove non-specifically bound proteins. The specifically-bound secreted protein is then released from the column and recovered using standard techniques. These procedures are well known in the art.

In an alternative protein purification scheme, the identified nucleic acid sequence of interest or portion thereof can be incorporated into expression vectors designed for use in purification schemes employing chimeric polypeptides. In such strategies the coding sequence of the identified nucleic acid sequence of interest or portion thereof is inserted in-frame with the gene encoding the other half of the chimera. The other half of the chimera can be maltose binding protein (MBP) or a nickel binding polypeptide encoding sequence. A chromatography matrix having antibody to MBP or nickel attached thereto is then used to purify the chimeric protein. Protease cleavage sites can be engineered between the MBP gene or the nickel binding polypeptide and the identified expected gene of interest, or portion thereof. Thus, the two polypeptides of the chimera can be separated from one another by protease digestion.

One useful expression vector for generating maltose binding protein fusion proteins is pMAL (New England Biolabs), which encodes the malE gene. In the pMal protein fusion system, the cloned gene is inserted into a pMal vector downstream from the malE gene. This results in the expression of an MBP-fusion protein. The fusion protein is purified by affinity chromatography. These techniques as described are well known to those skilled in the art of molecular biology. An Example describing in detail the generation of monoclonal and polyclonal antibodies appears below.

### EXAMPLE 17

### Production of an Antibody to an Isolated Staphylococcus aureus or Enterococcus faecalis Protein

Substantially pure protein or polypeptide is isolated from the transformed cells as described in Example 16. The concentration of protein in the final preparation is adjusted, for example, by concentration on a 10,000 molecular weight cut off AMICON filter device (Millipore, Bedford, Mass.), to the level of a few micrograms/ml. Monoclonal or polyclonal antibody to the protein can then be prepared as follows:

Monoclonal antibody to epitopes of any of the peptides identified and isolated as described can be prepared from murine hybridomas according to the classical method of Kohler, G. and Milstein, C., Nature 256:495 (1975) or any of the well-known derivative methods thereof. Briefly, a mouse is repetitively inoculated with a few micrograms of the selected protein or peptides derived therefrom over a period of a few weeks. The mouse is then sacrificed, and the antibody-producing cells of the spleen isolated. The spleen cells are fused by means of polyethylene glycol with mouse myeloma cells, and the excess unfused cells destroyed by growth of the system on selective media comprising aminopterin (HAT media). The successfully-fused cells are diluted and aliquots of the dilution placed in wells of a microtiter plate where growth of the culture is continued. Antibody-producing clones are identified by detection of antibody in the supernatant fluid of the wells by immunoassay procedures, such as ELISA, as described by Engvall, E., “Enzyme immunoassay ELISA and EMIT,” Meth. Enzymol. 70:419 (1980), and derivative methods thereof. Selected positive clones can be expanded and their monoclonal antibody product harvested for use. Detailed procedures for monoclonal antibody production are described in Davis, L. et al. Basic Methods in Molecular Biology Elsevier, New York. Section 21-2.

Polyclonal antiserum containing antibodies to heterogeneous epitopes of a single protein or a peptide can be prepared by immunizing suitable animals with the expressed protein or peptides derived therefrom described above, which can be unmodified or modified to enhance immunogenicity. Effective polyclonal antibody production is affected by many factors related both to the antigen and the host species. For example, small molecules tend to be less immunogenic than larger molecules and can require the use of carriers and adjuvant. Also, host animals vary in response to site of inoculations and dose, with both inadequate or excessive doses of antigen resulting in low titer antisera. Small doses (ng level) of antigen administered at multiple intradermal sites appears to be most reliable. An effective immunization protocol for rabbits can be found in Vaitukaitis, J. et al. J. Clin. Endocrinol. Metab. 33:988-991 (1971).

Booster injections can be given at regular intervals, and antiserum harvested when antibody titer thereof, as determined semi-quantitatively, for example, by double immunodiffusion in agar against known concentrations of the antigen, begins to fall. See, for example, Ouchterlony, O. et al., Chap. 19 in: Handbook of Experimental Immunology D. Wier (ed) Blackwell (1973). Plateau concentration of antibody is usually in the range of 0.1 to 0.2 mg/ml of serum (about 12:M). Affinity of the antisera for the antigen is determined by preparing competitive binding curves, as described, for example, by Fisher, D., Chap. 42 in: Manual of Clinical Immunology, 2d Ed. (Rose and Friedman, Eds.) Amer. Soc. For Microbiol., Washington, D.C. (1980).

Antibody preparations prepared according to either protocol are useful in quantitative immunoassays which determine concentrations of antigen-bearing substances in biological samples; they are also used semi-quantitatively or qualitatively to identify the presence of antigen in a biological sample. The antibodies can also be used in therapeutic compositions for killing bacterial cells expressing the protein. The next section describes how to make and use an in vivo system for performing in vivo analysis on bacterial strains containing regulatable essential genes and for screening compounds that inhibit bacterial proliferation.

The example below describes the an in vivo system for analyzing the growth characteristics of S. aureus and Enterococcus faecalis that contain regulatable essential genes.

### EXAMPLE 18

### In vivo Models for Analyzing the Regulation of Essential Genes and Screening Therapeutic Compounds

Whether a given gene is essential for a bacterium growing in nature (in vivo) or whether a gene is required for infection of a host organism can be determined by constructing a conditional allele of that gene which is suitable for animal testing. Expression of an antisense RNA molecule complementary to a candidate gene can, by decreasing synthesis of the candidate gene product, constitute a conditional allele that is suitable for animal testing. Candidate genes essential in vivo or required for virulence can be tested by preparing bacterial strains using the EGI, antisense, promoter replacement, operator insertion, and random promoter insertion techniques. Preferably, the constructs used to prepare these bacterial strains contain an expression vector under control of a fusion promoter described herein. Animals infected by these bacterial strains can be used to evaluate the ability to modulate bacterial proliferation in vivo by adding or decreasing the concentration of inducer and can be used to screen compounds in vivo to validate their therapeutic properties.

Stable transformation of Staphylococcus aureus or Enterococcus faecalis with constructs prepared as described herein can be accomplished by electroporation and selection for clones resistant to 15 μg/ml chloramphenicol in the case of Staphylococcus aureus of 20 μg/ml erythromycin in the case of Enterococcus faecalis. Preferably, whether each construct is essential is verified in vitro using induction in medium containing various amounts of inducer or an analog thereof, prior to introducing the cells into animals.

For the thigh infection model, animals are rendered neutropenic chemically prior to the start of the experiment. By one approach, the inoculum will consist of 10⁵ to 10⁶ cells of a normally virulent Gram-positive organism expressing antisense to an essential gene such as that for gyrB under control of a fusion promoter described herein. These cells are injected to one thigh of a suitable animal (e.g., mice). Most microorganisms attain a logarithmic growth 2 hours after inoculation. Treated animals receive therapy from 2 hours post inoculation up to 24 hours. A typical efficacy study consists of 2 control groups and 5 treatment groups, each being treated with different doses of inducer. With 15 animals per group, 105 mice are used per study. The control sets are given intraperitoneal injections of saline, which will not induce expression of the fusion promoter. The experimental animals are given the inducer (e.g. xylose, tetracycline, IPTG, maltose, absence of tryptophan, or temperature change) in intraperitoneal injections to induce expression of the antisense promoter. Alternatively, the promoter can be induced by intravenous infusion of inducer at sub-toxic levels.

The end point used to follow the infection process is viable bacterial counts per thigh. The controls in which the antisense RNA is not induced will fail to impede growth of the Gram-positive organism and thus a logarithmic increase in viable bacteria will occur. The Gram-positive cells recovered from the site of infection should be viable until antisense expression is subsequently induced. This will demonstrate that the plasmid is still patent. However, in animals receiving the xylose injections, expression of the antisense RNA will occur, the essential gene or gene product will be compromised, and the Gram-positive cells infecting the mice will not multiply. Accordingly, fewer viable cells will be recovered from the site of infection in the experimental animals. The Gram-positive cells from the induced mice will be recovered, if still present, and assayed as above to determine if the promoter and gene are still present and functional.

The sections below describe the use of fusion promoters in methods to discover compounds that inhibit bacterial proliferation.

### EXAMPLE 19

### Screening Chemical Libraries in Cells Sensitized with Antisense RNA Transcribed from Fusion Promoters

Having isolated and expressed bacterial proteins shown to be required for bacterial proliferation, the present invention further contemplates the use of these expressed target proteins in assays to screen libraries of compounds for potential drug candidates. The generation of chemical libraries is well known in the art. For example, combinatorial chemistry can be used to generate a library of compounds to be screened in the assays described herein. A combinatorial chemical library is a collection of diverse chemical compounds generated by either chemical synthesis or biological synthesis by combining a number of chemical “building block” reagents. For example, a linear combinatorial chemical library such as a polypeptide library is formed by combining amino acids in every possible combination to yield peptides of a given length. Millions of chemical compounds theoretically can be synthesized through such combinatorial mixings of chemical building blocks. For example, one commentator observed that the systematic, combinatorial mixing of 100 interchangeable chemical building blocks results in the theoretical synthesis of 100 million tetrameric compounds or 10 billion pentameric compounds. (Gallop et al., 1994, “Applications of Combinatorial Technologies to Drug Discovery, Background and Peptide Combinatorial Libraries” Journal of Medicinal Chemistry, 37: 1233-1250). Other chemical libraries known to those in the art may also be used, including natural product libraries.

Once generated, combinatorial libraries can be screened for compounds that possess desirable biological properties. For example, compounds which may be useful as drugs or to develop drugs would likely have the ability to bind to the target protein identified, expressed and purified as discussed above. Further, if the identified target protein is an enzyme, candidate compounds would likely interfere with the enzymatic properties of the target protein. For example, the enzymatic function of a target protein may be to serve as a protease, nuclease, phosphatase, dehydrogenase, transporter protein, transcriptional enzyme, and any other type of enzyme known or unknown. Thus, the present invention contemplates using the protein products described above to screen combinatorial chemical libraries.

In one example, the target protein is a serine protease and the substrate of the enzyme is known. The present example is directed towards the analysis of libraries of compounds to identify compounds that function as inhibitors of the target enzyme. First, a library of small molecules is generated using methods of combinatorial library formation well known in the art. U.S. Pat. Nos. 5,463,564 and 5,574,656, to Agrafiotis, et al., entitled “System and Method of Automatically Generating Chemical Compounds with Desired Properties,” the disclosures of which are incorporated herein by reference in their entireties, are two such teachings. Then the library compounds are screened to identify those compounds that possess desired structural and functional properties. U.S. Pat. No. 5,684,711, the disclosure of which is incorporated herein by reference in its entirety, also discusses a method for screening libraries.

To illustrate the screening process, the target polypeptide and chemical compounds of the library are combined with one another and permitted to interact with one another. A labeled substrate is added to the incubation. The label on the substrate is such that a detectable signal is emitted from metabolized substrate molecules. The emission of this signal permits one to measure the effect of the combinatorial library compounds on the enzymatic activity of target enzymes by comparint it to the signal emitted in the absence of combinatorial library compounds. The characteristics of each library compound are encoded so that compounds demonstrating activity against the enzyme can be analyzed and features common to the various compounds identified can be isolated and combined into future iterations of libraries.

Once a library of compounds is screened, subsequent libraries are generated using those chemical building blocks that possess the features shown in the first round of screen to have activity against the target enzyme. Using this method, subsequent iterations of candidate compounds will possess more and more of those structural and functional features required to inhibit the function of the target enzyme, until a group of enzyme inhibitors with high specificity for the enzyme can be found. These compounds can then be further tested for their safety and efficacy as antibiotics for use in mammals.

It will be readily appreciated that this particular screening methodology is exemplary only. Other methods are well known to those skilled in the art. For example, a wide variety of screening techniques are known for a large number of naturally-occurring targets when the biochemical function of the target protein is known. For example, some techniques involve the generation and use of small peptides to probe and analyze target proteins both biochemically and genetically in order to identify and develop drug leads. Such techniques include the methods described in PCT publications No. WO9935494, WO9819162, WO9954728, the disclosures of which are incorporated herein by reference in their entireties.

Current cell-based assays used to identify or to characterize compounds for drug discovery and development frequently depend on detecting the ability of a test compound to modulate the activity of a target molecule located within a cell or located on the surface of a cell. Most often such target molecules are proteins such as enzymes, receptors and the like. However, target molecules may also include other molecules such as DNAs, lipids, carbohydrates and RNAs including messenger RNAs, ribosomal RNAs, tRNAs and the like. A number of highly sensitive cell-based assay methods are available to those of skill in the art to detect binding and interaction of test compounds with specific target molecules. However, these methods are generally not highly effective when the test compound binds to or otherwise interacts with its target molecule with moderate or low affinity. In addition, the target molecule may not be readily accessible to a test compound in solution, such as when the target molecule is located inside the cell or within a cellular compartment such as the periplasm of a bacterial cell. Thus, current cell-based assay methods are limited in that they are not effective in identifying or characterizing compounds that interact with their targets with moderate to low affinity or compounds that interact with targets that are not readily accessible.

The cell-based assay methods of the present invention have substantial advantages over current cell-based assays. These advantages derive from the use of sensitized cells in which the level or activity of at least one proliferation-required gene product (the target molecule) has been specifically reduced to the point where the presence or absence of its function becomes a rate-determining step for cellular proliferation. In the cell-based assays of the present invention, the level or activity of a proliferation-required gene product is reduced by transcribing an antisense nucleic acid complementary to at least a portion of the nucleic acid encoding the gene product from one of the fusion promoters described herein. Such sensitized cells become much more sensitive to compounds that are active against the affected target molecule. Thus, cell-based assays of the present invention are capable of detecting compounds exhibiting low or moderate potency against the target molecule of interest because such compounds are substantially more potent on sensitized cells than on non-sensitized cells. The effect may be such that a test compound may be two to several times more potent, at least 10 times more potent, at least 20 times more potent, at least 50 times more potent, at least 100 times more potent, at least 1000 times more potent, or even more than 1000 times more potent when tested on the sensitized cells as compared to the non-sensitized cells.

Due in part to the increased appearance of antibiotic resistance in pathogenic microorganisms and to the significant side-effects associated with some currently used antibiotics, novel antibiotics acting at new targets are highly sought after in the art. Yet, another limitation in the current art related to cell-based assays is the problem of repeatedly identifying hits against the same kinds of target molecules in the same limited set of biological pathways. This may occur when compounds acting at such new targets are discarded, ignored or fail to be detected because compounds acting at the “old” targets are encountered more frequently and are more potent than compounds acting at the new targets. As a result, the majority of antibiotics in use currently interact with a relatively small number of target molecules within an even more limited set of biological pathways.

The use of sensitized cells comprising the fusion promoters of the current invention provides a solution to the above problem in two ways. First, desired compounds acting at a target of interest, whether a new target or a previously known but poorly exploited target, can now be detected above the “noise” of compounds acting at the “old” targets due to the specific and substantial increase in potency of such desired compounds when tested on the sensitized cells of the current invention. Second, the methods used to sensitize cells to compounds acting at a target of interest may also sensitize these cells to compounds acting at other target molecules within the same biological pathway. For example, expression of an antisense molecule to a gene encoding a ribosomal protein is expected to sensitize the cell to compounds acting at that ribosomal protein and may also sensitize the cells to compounds acting at any of the ribosomal components (proteins or rRNA) or even to compounds acting at any target which is part of the protein synthesis pathway. Thus an important advantage of the present invention is the ability to reveal new targets and pathways that were previously not readily accessible to drug discovery methods.

Sensitized cells of the present invention are prepared by reducing the activity or level of a target molecule. The target molecule may be a gene product, such as an RNA or polypeptide produced from the proliferation-required nucleic acids described herein. Alternatively, the target may be a gene product such as an RNA or polypeptide which is produced from a sequence within the same operon as the proliferation-required nucleic acids described herein. In addition, the target may be an RNA or polypeptide in the same biological pathway as the proliferation-required nucleic acids described herein. Such biological pathways include, but are not limited to, enzymatic, biochemical and metabolic pathways as well as pathways involved in the production of cellular structures such the cell wall.

Current methods employed in the arts of medicinal and combinatorial chemistries are able to make use of structure-activity relationship information derived from testing compounds in various biological assays including direct binding assays and cell-based assays. Occasionally compounds are directly identified in such assays that are sufficiently potent to be developed as drugs. More often, initial hit compounds exhibit moderate or low potency. Once a hit compound is identified with low or moderate potency, directed libraries of compounds are synthesized and tested in order to identify more potent leads. Generally these directed libraries are combinatorial chemical libraries consisting of compounds with structures related to the hit compound but containing systematic variations including additions, subtractions and substitutions of various structural features. When tested for activity against the target molecule, structural features are identified that either alone or in combination with other features enhance or reduce activity. This information is used to design subsequent directed libraries containing compounds with enhanced activity against the target molecule. After one or several iterations of this process, compounds with substantially increased activity against the target molecule are identified and may be further developed as drugs. This process is facilitated by use of the sensitized cells of the present invention since compounds acting at the selected targets exhibit increased potency in such cell-based assays, thus; more compounds can now be characterized providing more useful information than would be obtained otherwise.

Thus, it is now possible using cell-based assays of the present invention to identify or characterize compounds that previously would not have been readily identified or characterized including compounds that act at targets that previously were not readily exploited using cell-based assays. The process of evolving potent drug leads from initial hit compounds is also substantially improved by the cell-based assays of the present invention because, for the same number of test compounds, more structure-function relationship information is likely to be revealed.

The method of sensitizing a cell entails selecting a suitable gene or operon. A suitable gene or operon is one whose expression is required for the proliferation of the cell to be sensitized. The next step is to introduce into the cells to be sensitized, an antisense RNA capable of hybridizing to the suitable gene or operon or to the RNA encoded by the suitable gene or operon. Introduction of the antisense RNA can be in the form of an expression vector in which antisense RNA is produced under the control of a fusion promoter as described herein. The amount of antisense RNA produced is limited by varying the inducer concentration to which the cell is exposed and thereby varying the activity of the promoter driving transcription of the antisense RNA. Thus, cells are sensitized by exposing them to an inducer concentration that results in a sub-lethal level of antisense RNA expression.

In one embodiment of the cell-based assays, an antisense nucleic acid that is complementary to a proliferation-required gene from a Gram-positive organism is used to inhibit the production of a proliferation-required protein. Expression vectors which contain an antisense nucleic acid complementary to identified genes required for proliferation operably linked to a fusion promoter of the present invention are used to limit the concentration of a proliferation-required protein without severely inhibiting growth. To achieve that goal, a growth inhibition dose curve of inducer is calculated by plotting various doses of inducer against the corresponding growth inhibition caused by the antisense expression. From this curve, various percentages of antisense induced growth inhibition, from 1 to 100% can be determined. If the fusion promoter contained in the expression vector contains a xylO linked to one of the promoters of SEQ ID NO.: 36-45, transcription in Staphylococcus aureus and Enterococcus faecalis will be regulatable by the xyl repressor and expression from the promoter can be induced with xylose. Similarly, IPTG, xylose, tetracycline, maltose, absence of trp and temperature inducible promoters may be used. For example, the highest concentration of the inducer that does not reduce the growth rate significantly can be estimated from the curve. Cellular proliferation can be monitored by growth medium turbidity via OD measurements. In another example, the concentration of inducer that reduces growth by 25% can be predicted from the curve. In still another example, a concentration of inducer that reduces growth by 50% can be calculated. Additional parameters such as colony forming units (cfu) can be used to measure cellular viability.

Cells to be assayed are exposed to the above-determined concentrations of inducer. The presence of the inducer at this sub-lethal concentration reduces the amount of the proliferation required gene product to the lowest amount in the cell that will support growth. Cells grown in the presence of this concentration of inducer are therefore specifically more sensitive to inhibitors of the proliferation-required protein or RNA of interest or to inhibitors of proteins or RNAs in the same biological pathway as the proliferation-required protein or RNA of interest but not to inhibitors of unrelated proteins or RNAs.

Cells pretreated with sub-inhibitory concentrations of inducer and thus containing a reduced amount of proliferation-required target gene product are then used to screen for compounds that reduce cell growth. The sub-lethal concentration of inducer may be any concentration consistent with the intended use of the assay to identify candidate compounds to which the cells are more sensitive. For example, the sub-lethal concentration of the inducer may be such that growth inhibition is at least about 5%, at least about 8%, at least about 10%, at least about 20%, at least about 30%, at least about 40%, at least about 50%, at least about 60% at least about 75%, or more. Cells which are pre-sensitized using the preceding method are more sensitive to inhibitors of the target protein because these cells contain less target protein to inhibit than wild-type cells.

It will be appreciated that the cell-based assays described above may be implemented in Staphylococcus aureus and Enterococcus faecalis using proliferation-inhibiting antisense sequences under the control of a fusion promoter described herein. It will also be appreciated that the above cell-based assays employing the fusion promoters described herein can be implemented in other Gram-positive organims, including but not limited to, Bacillus anthracis, Clostridium botulinum, Clostridium difficile, Clostridium perfringens, Clostridium teteni, Corynebacterium diptheriae, Enterococcus faecalis, Enterococcus faecium, Lactococcus lactis, Listeria monocytogenes, Mycobacterium leprae, Mycobacterium tuberculosis, Nocardia asteroides, Staphylococcus aureus, Staphylococcus epidermidis, Staphylococcus xylosis, Streptococcus pneumoniae, Streptococcus mutans or any species falling within the genera of any of the above species.

In another embodiment of the cell-based assays of the present invention, the level or activity of a proliferation required gene product is reduced using a mutation, such as a temperature sensitive mutation, in the proliferation-required sequence and an antisense nucleic acid complementary to the proliferation-required sequence which is transcribed from one of the fusion promoters described herein. Growing the cells at an intermediate temperature between the permissive and restrictive temperatures of the temperature sensitive mutant where the mutation is in a proliferation-required gene produces cells with reduced activity of the proliferation-required gene product. The antisense RNA complementary to the proliferation-required sequence further reduces the activity of the proliferation required gene product. Drugs that may not have been found using either the temperature sensitive mutation or the antisense nucleic acid alone may be identified by determining whether cells in which expression of the antisense nucleic acid has been induced and which are grown at a temperature between the permissive temperature and the restrictive temperature are substantially more sensitive to a test compound than cells in which expression of the antisense nucleic acid has not been induced and which are grown at a permissive temperature. Also drugs found previously from either the antisense nucleic acid alone or the temperature sensitive mutation alone may have a different sensitivity profile when used in cells combining the two approaches, and that sensitivity profile may indicate a more specific action of the drug in inhibiting one or more activities of the gene product.

Temperature sensitive mutations may be located at different sites within the gene and correspond to different domains of the protein. For example, the dnaB gene of Escherichia coli encodes the replication fork DNA helicase. DnaB has several domains, including domains for oligomerization, ATP hydrolysis, DNA binding, interaction with primase, interaction with DnaC, and interaction with DnaA [(Biswas, E. E. and Biswas, S. B. 1999, “Mechanism and DnaB helicase of Escherichia coli: structural domains involved in ATP hydrolysis, DNA binding, and oligomerization” Biochem. 38: 10919-10928; Hiasa, H. and Marians, K. J. 1999, “Initiation of bidirectional replication at the chromosomal origin is directed by the interaction between helicase and primase” J. Biol. Chem. 274: 27244-27248; San Martin, C., et al. 1998, “Three-dimensional reconstructions from cryoelectron microscopy images reveal an intimate complex between helicase DnaB and its loading partner DnaC” Structure 6:501-9; Sutton, M. D. et al. 1998. “Escherichia coli DnaA protein. The N-terminal domain and loading of DnaB helicase at the E. coli chromosomal origin” J. Biol. Chem. 273: 34255-62.), the disclosures of which are incorporated herein by reference in their entireties]. Temperature sensitive mutations in different domains of DnaB confer different phenotypes at the restrictive temperature, which include either an abrupt stop or slow stop in DNA replication with or without DNA breakdown (Wechsler, J. A. and Gross, J. D. 1971. “Escherichia coli mutants temperature-sensitive for DNA synthesis” Mol. Gen. Genetics 113: 273-284, the disclosure of which is incorporated herein by reference in its entirety) and termination of growth or cell death. Combining the use of temperature sensitive mutations in the dnaB gene that cause cell death at the restrictive temperature with an antisense to the dnaB gene could lead to the discovery of very specific and effective inhibitors of one or a subset of activities exhibited by DnaB.

It will be appreciated that the above method may be performed with any mutation which reduces but does not eliminate the activity or level of the gene product which is required for proliferation.

When screening for antimicrobial agents against a gene product required for proliferation, growth inhibition of cells containing a limiting amount of that proliferation-required gene product can be assayed. Growth inhibition can be measured by directly comparing the amount of growth, measured by the optical density of the growth medium, between an experimental sample and a control sample. Alternative methods for assaying cell proliferation include measuring the activity of any of a variety of reporter genes, various enzymatic activity assays, and other methods well known in the art.

It will be appreciated that the above method may be performed in solid phase, liquid phase or a combination of the two. For example, cells grown on nutrient agar containing the inducer of the antisense construct may be exposed to compounds spotted onto the agar surface. A compound's effect may be judged from the diameter of the resulting killing zone, the area around the compound application point in which cells do not grow. Multiple compounds may be transferred to agar plates and simultaneously tested using automated and semi-automated equipment including but not restricted to multi-channel pipettes (for example the Beckman Multimek) and multi-channel spotters (for example the Genomic Solutions Flexys). In this way multiple plates and thousands to millions of compounds may be tested per day.

The compounds may also be tested entirely in liquid phase using microtiter plates as described below. Liquid phase screening may be performed in microtiter plates containing 96, 384, 1536 or more wells per microtiter plate to screen multiple plates and thousands to millions of compounds per day. Automated and semi-automated equipment may be used for addition of reagents (for example cells and compounds) and determination of cell density.

The examples below describe cell-based assays that exploit the fusion promoters described herein to sensitize cells and allow for the discovery of a diverse class of antibiotics. Example 20 describes experiments performed in E. coli, which verify the effectiveness of the cell-based approach described above.

### EXAMPLE 20

### Cell-Based Assay Using Antisense RNA Complementary to Genes Encoding Ribosomal Proteins

The effectiveness of the above cell-based assay was validated using constructs expressing antisense RNA to the proliferation required E. coli genes rplL, rplJ, and rplW encoding ribosomal proteins L7/L12, L10 and L23 respectively. These proteins are part of the protein synthesis apparatus of the cell and as such are required for proliferation. These constructs were used to test the effect of antisense expression on cell sensitivity to antibiotics known to bind to the ribosome and thereby inhibit protein synthesis. Constructs expressing antisense RNA to several other genes (elaD, visC, yohH, and atpE/B), the products of which are not involved in protein synthesis were used for comparison.

First, pLex5BA (Krause et al. 1997. J. Mol. Biol. 274: 365, the disclosure of which is incorporated herein by reference in its entirety) expression vectors containing antisense constructs to either rplW or to elaD were introduced into separate E. coli cell populations. Vector introduction is a technique well known to those of ordinary skill in the art. The expression vectors of this example contain IPTG inducible promoters that drive the expression of the antisense RNA in the presence of the inducer. However, those skilled in the art will appreciate that other inducible promoters may also be used. Suitable expression vectors are also well known in the art. The E. coli antisense clones to genes encoding ribosomal proteins L7/L12, L10 and L23 were used to test the effect of antisense expression on cell sensitivity to the antibiotics known to bind to these proteins. Expression vectors containing antisense to either the genes encoding L7/L12 and L10 or L23 were introduced into separate E. coli cell populations.

The cell populations were exposed to a range of IPTG concentrations in liquid medium to obtain the growth inhibitory dose curve for each clone. First, seed cultures were grown to a particular turbidity that is measured by the optical density (OD) of the growth solution. The OD of the solution is directly related to the number of bacterial cells contained therein. Subsequently, sixteen 200 ul liquid medium cultures were grown in a 96 well microtiter plate at 37° C. with a range of IPTG concentrations in duplicate two-fold serial dilutions from 1600 uM to 12.5 uM (final concentration). Additionally, control cells were grown in duplicate without IPTG. These cultures were started from equal amounts of cells derived from the same initial seed culture of a clone of interest. The cells were grown for up to 15 hours and the extent of growth was determined by measuring the optical density of the cultures at 600 nm. When the control culture reached mid-log phase the percent growth (relative to the control culture) for each of the IPTG containing cultures was plotted against the log concentrations of IPTG to produce a growth inhibitory dose response curve for the IPTG. The concentration of IPTG that inhibits cell growth to 50% (IC₅₀) as compared to the 0 mM IPTG control (0% growth inhibition) was then calculated from the curve. Under these conditions, an amount of antisense RNA was produced that reduced the expression levels of rplW and elaD to a degree such that growth was inhibited by 50%.

Alternative methods of measuring growth are also contemplated. Examples of these methods include measurements of proteins, the expression of which is engineered into the cells being tested and can readily be measured. Examples of such proteins include green fluorescent protein (GFP) and various enzymes.

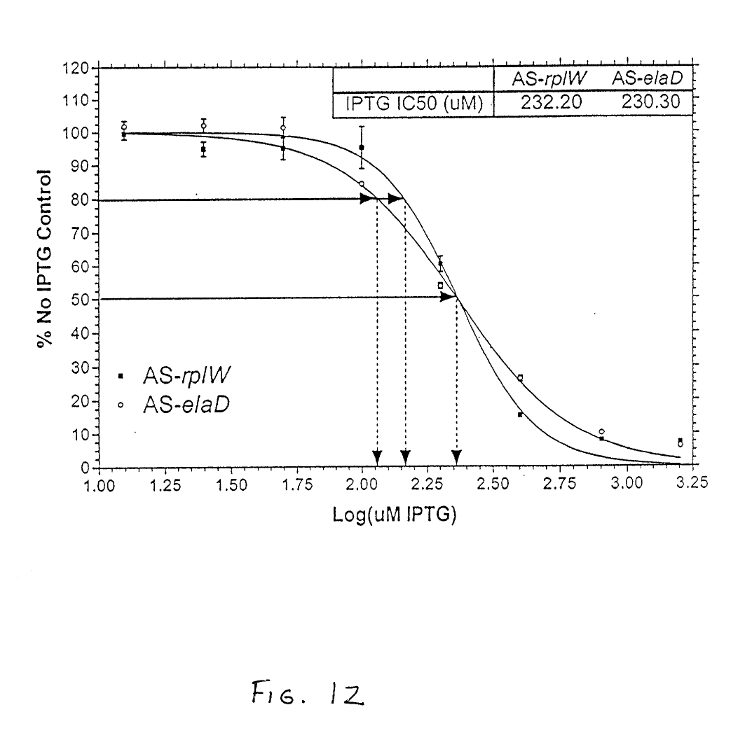

Cells were pretreated with the selected concentration of IPTG and then used to test the sensitivity of cell populations to tetracycline, erythromycin and other protein synthesis inhibitors. FIG. 12 is an IPTG dose response curve in E. coli transformed with an IPTG-inducible plasmid containing either an antisense clone to the E. coli rplW gene (AS-rplW) which encodes ribosomal protein L23 which is required for protein synthesis and essential for cell proliferation, or an antisense clone to the elaD (AS-elaD) gene which is not known to be involved in protein synthesis and which is also essential for proliferation.

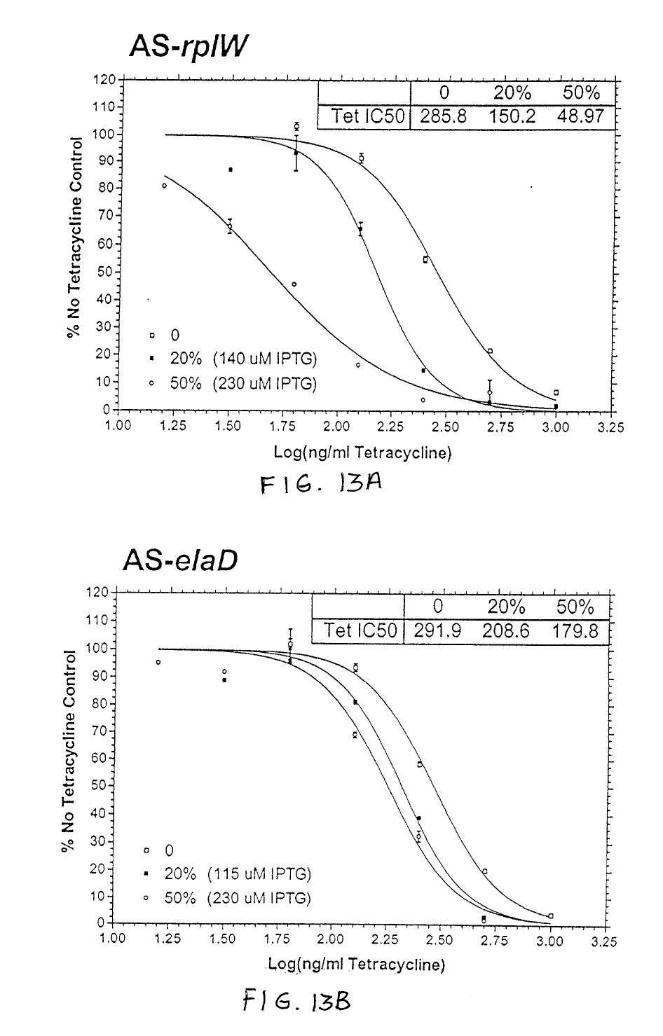

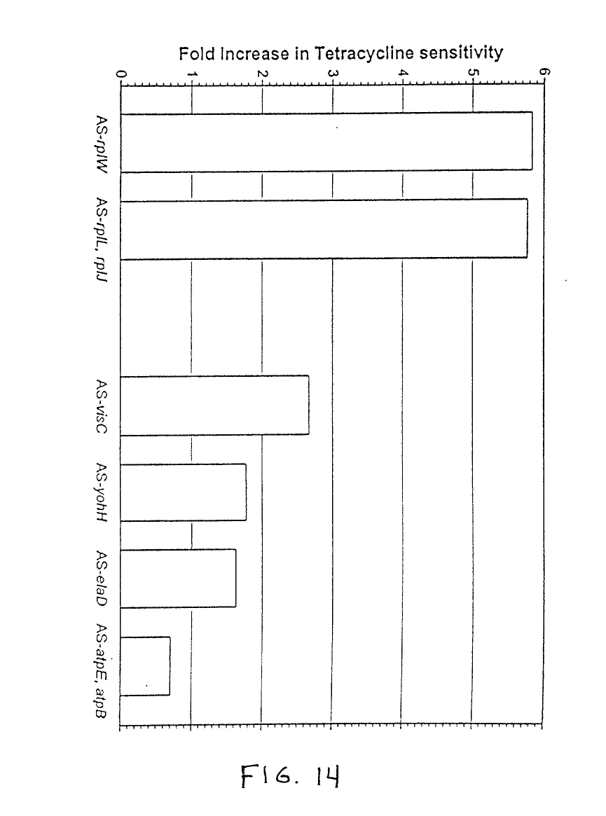

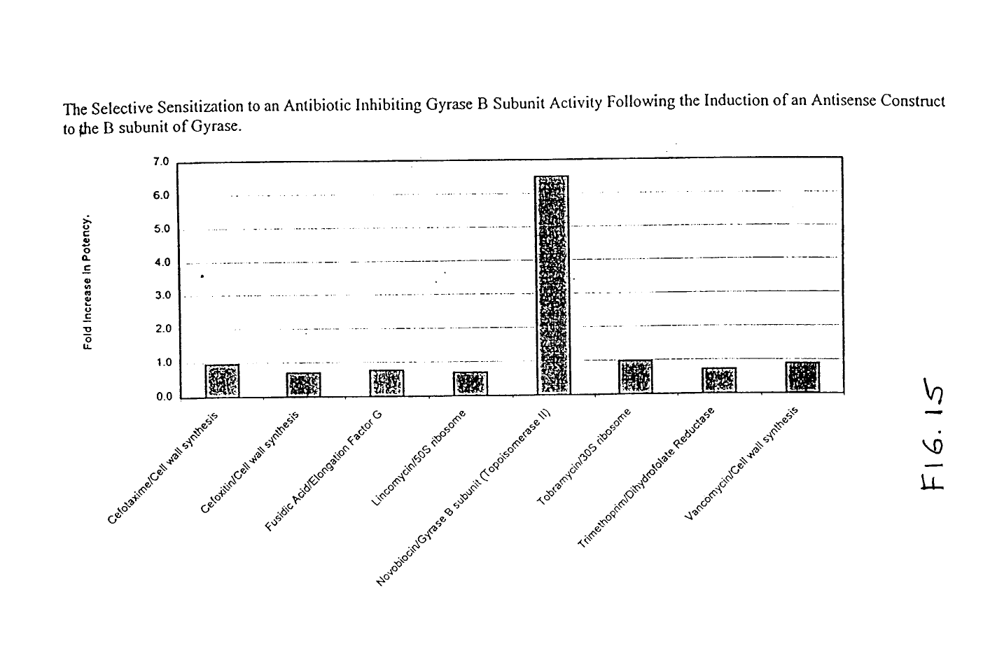

An example of a tetracycline dose response curve is shown in FIGS. 13A and 13B for the rplW and elaD genes, respectively. Cells were grown to log phase and then diluted into media alone or media containing IPTG at concentrations which give 20% and 50% growth inhibition as determined by IPTG dose response curves. After 2.5 hours, the cells were diluted to a final OD₆₀₀ of 0.002 into 96 well plates containing (1) +/−IPTG at the same concentrations used for the 2.5 hour pre-incubation; and (2) serial two-fold dilutions of tetracycline such that the final concentrations of tetracycline range from 1 μg/ml to 15.6 ng/ml and 0 μg/ml. The 96 well plates were incubated at 37° C. and the OD₆₀₀ was read by a plate reader every 5 minutes for up to 15 hours. For each IPTG concentration and the no IPTG control, tetracycline dose response curves were determined when the control (absence of tetracycline) reached 0.1 OD₆₀₀. To compare tetracycline sensitivity with and without IPTG, tetracycline IC₅₀ₛ were determined from the dose response curves (FIGS. 13A and 13B). Cells with reduced levels of L23 (AS-rplW) showed increased sensitivity to tetracycline (FIG. 13A) as compared to cells with reduced levels of the elaD gene product (AS-elaD) (FIG. 13B). FIG. 14 shows a summary bar chart in which the ratios of tetracycline IC₅₀ₛ determined in the presence of IPTG which gives 50% growth inhibition versus tetracycline IC₅₀ₛ determined without IPTG (fold increase in tetracycline sensitivity) were plotted. Cells with reduced levels of either L7/L12 (encoded by genes rplL, rplJ) or L23 (encoded by the rplW gene) showed increased sensitivity to tetracycline (FIG. 14). Cells expressing antisense to genes not known to be involved in protein synthesis (AS-atpB/E, AS-visC, AS-elaD, AS-yohH) did not show the same increased sensitivity to tetracycline, validating the specificity of this assay (FIG. 14).

In addition to the above, it has been observed in initial experiments that clones expressing antisense RNA to genes involved in protein synthesis (including genes encoding ribosomal proteins L7/L12 & L10, L7/L12 alone, L22, and L18, as well as genes encoding rRNA and Elongation Factor G) have increased sensitivity to the macrolide, erythromycin, whereas clones expressing antisense to the non-protein synthesis genes elaD, atpB/E and visC do not. Furthermore, the clone expressing antisense to rplL and rplJ does not show increased sensitivity to nalidixic acid and ofloxacin, antibiotics which do not inhibit protein synthesis.

The results with the ribosomal protein genes rplL, rplJ, and rplW as well as the initial results using various other antisense clones and antibiotics show that limiting the concentration of an antibiotic target makes cells more sensitive to the antimicrobial agents that specifically interact with that protein. The results also show that these cells are sensitized to antimicrobial agents that inhibit the overall function in which the protein target is involved but are not sensitized to antimicrobial agents that inhibit other functions.

It will be appreciated that the cell-based assays described above may be implemented in Staphylococcus aureus and Enterococcus faecalis using proliferation-inhibiting antisense sequences under the control of a fusion promoter described herein. It will also be appreciated that the above cell-based assays employing the fusion promoters described herein can be implemented in other Gram-positive organims, including but not limited to, Bacillus anthracis, Clostridium botulinum, Clostridium difficile, Clostridium perfringens, Clostridium teteni, Corynebacterium diptheriae, Enterococcus faecalis, Enterococcus faecium, Lactococcus lactis, Listeria monocytogenes, Mycobacterium leprae, Mycobacterium tuberculosis, Nocardia asteroides, Staphylococcus aureus, Staphylococcus epidermidis, Staphylococcus xylosis, Streptococcus pneumoniae, Streptococcus mutans or any species falling within the genera of any of the above species.

The cell-based assay described above may also be used to identify the biological pathway in which a proliferation-required nucleic acid or its gene product lies. In such methods, cells transcribing a sub-lethal level of antisense to a target proliferation-required nucleic acid from the fusion promoters described herein and control cells in which expression of the antisense has not been induced are contacted with a panel of antibiotics known to act in various pathways. If the antibiotic acts in the pathway in which the target proliferation-required nucleic acid or its gene product lies, cells in which expression of the antisense has been induced will be more sensitive to the antibiotic than cells in which expression of the antisense has not been induced.

As a control, the results of the assay may be confirmed by contacting a panel of cells expressing antisense nucleic acids to many different proliferation-required genes including the target proliferation-required gene. If the antibiotic is acting specifically, heightened sensitivity to the antibiotic will be observed only in the cells expressing antisense to a target proliferation-required gene (or cells expressing antisense to other proliferation-required genes in the same pathway as the target proliferation-required gene) but will not be observed generally in all cells expressing antisense to proliferation-required genes.

It will be appreciated that the cell-based assays described above may be implemented in Staphylococcus aureus and Enterococcus faecalis using proliferation-inhibiting antisense sequences under the control of a fusion promoter described herein. It will also be appreciated that the above cell-based assays employing the fusion promoters described herein can be implemented in other Gram-positive organims, including but not limited to, Bacillus anthracis, Clostridium botulinum, Clostridium difficile, Clostridium perfringens, Clostridium teteni, Corynebacterium diptheriae, Enterococcus faecalis, Enterococcus faecium, Lactococcus lactis, Listeria monocytogenes, Mycobacterium leprae, Mycobacterium tuberculosis, Nocardia asteroides, Staphylococcus aureus, Staphylococcus epidermidis, Staphylococcus xylosis, Streptococcus pneumoniae, Streptococcus mutans or any species falling within the genera of any of the above species.

Similarly, the above method may be used to determine the pathway on which a test compound, such as a test antibiotic acts. A panel of cells, each of which transcribes antisense to a proliferation-required nucleic acid in a known pathway from one of the fusion promoters described herein, is contacted with a compound for which it is desired to determine the pathway on which it acts. The sensitivity of the panel of cells to the test compound is determined in cells in which expression of the antisense has been induced and in control cells in which expression of the antisense has not been induced. If the test compound acts on the pathway on which an antisense nucleic acid acts, cells in which expression of the antisense has been induced will be more sensitive to the compound than cells in which expression of the antisense has not been induced. In addition, control cells in which expression of antisense to proliferation-required genes in other pathways has been induced will not exhibit heightened sensitivity to the compound. In this way, the pathway on which the test compound acts may be determined.

It will be appreciated that the cell-based assays described above may be implemented in Staphylococcus aureus and Enterococcus faecalis using proliferation-inhibiting antisense sequences under the control of a fusion promoter described herein. It will also be appreciated that the above cell-based assays employing the fusion promoters described herein can be implemented in other Gram-positive organims, including but not limited to, Bacillus anthracis, Clostridium botulinum, Clostridium difficile, Clostridium perfringens, Clostridium teteni, Corynebacterium diptheriae, Enterococcus faecalis, Enterococcus faecium, Lactococcus lactis, Listeria monocytogenes, Mycobacterium leprae, Mycobacterium tuberculosis, Nocardia asteroides, Staphylococcus aureus, Staphylococcus epidermidis, Staphylococcus xylosis, Streptococcus pneumoniae, Streptococcus mutans or any species falling within the genera of any of the above species.

The Example below provides one method for performing such assays.

### EXAMPLE 21

### Identification of the Pathway in Which a Proliferation-Required Gene Lies or the Pathway on which an Antibiotic Acts

A. Preparation of Bacterial Stocks for Assay

To provide a consistent source of cells to screen, frozen stocks of host bacteria containing the desired antisense construct are prepared using standard microbiological techniques. For example, a single clone of the microorganism can be isolated by streaking out a sample of the original stock onto an agar plate containing nutrients for cell growth and an antibiotic for which the antisense construct contains a gene which confers resistance. After overnight growth an isolated colony is picked from the plate with a sterile needle and transferred to an appropriate liquid growth media containing the antibiotic required for maintenance of the plasmid. The cells are incubated at 30° C. to 37° C. with vigorous shaking for 4 to 6 hours to yield a culture in exponential growth. Sterile glycerol is added to 15% (volume to volume) and 100 μL to 500 μL aliquots are distributed into sterile cryotubes, snap frozen in liquid nitrogen, and stored at −80° C. for future assays.

B. Growth of Bacteria for Use in the Assay

A day prior to an assay, a stock vial is removed from the freezer, rapidly thawed (37° C. water bath) and a loop of culture is streaked out on an agar plate containing nutrients for cell growth and an antibiotic to which the antisense construct confers resistance. After overnight growth at 37° C., ten randomly chosen, isolated colonies are transferred from the plate (sterile inoculum loop) to a sterile tube containing 5 mL of appropriate medium containing the antibiotic to which the antisense vector confers resistance. After vigorous mixing to form a homogeneous cell suspension, the optical density of the suspension is measured at 600 nm (OD₆₀₀) and if necessary an aliquot of the suspension is diluted into a second tube of 5 mL, sterile, appropriate medium plus antibiotic to achieve an OD₆₀₀≦0.02 absorbance units. The culture is then incubated at 37° C. for 1-2 hrs with shaking until the OD₆₀₀ reaches OD 0.2-0.3. At this point the cells are ready to be used in the assay.

C. Selection of Media to be Used in Assay

Two-fold dilution series of the inducer are generated in culture media containing the appropriate antibiotic for maintenance of the antisense construct. Several media are tested side by side and three to four wells are used to evaluate the effects of the inducer at each concentration in each media. For example, LB broth, TBD broth, Muller-Hinton, or other media may be tested with the inducer xylose at the following concentrations, 5 mM, 10 mM, 20 mM, 40 mM, 80 mM, 120 mM and 160 mM. Equal volumes of test media-inducer and cells are added to the wells of a 384 well microtiter plate and mixed. The cells are prepared as described above and diluted 1:100 in the appropriate media containing the test antibiotic immediately prior to addition to the microtiter plate wells. For a control, cells are also added to several wells of each media that do not contain inducer, for example 0 mM xylose. Cell growth is monitored continuously by incubation at 37° C. in a microtiter plate reader monitoring the OD₆₀₀ of the wells over an 18-hour period. The percent inhibition of growth produced by each concentration of inducer is calculated by comparing the rates of logarithmic growth against that exhibited by cells growing in media without inducer. The medium yielding greatest sensitivity to inducer is selected for use in the assays described below.

D. Measurement of Test Antibiotic Sensitivity in the Absence of Antisense Construct Induction

Two-fold dilution series of antibiotics of known mechanism of action are generated in the culture media selected for further assay development that has been supplemented with the antibiotic used to maintain the construct. A panel of test antibiotics known to act on different pathways is tested side by side with three to four wells being used to evaluate the effect of a test antibiotic on cell growth at each concentration. Equal volumes of test antibiotic and cells are added to the wells of a 384 well microtiter plate and mixed. Cells are prepared as described above using the media selected for assay development supplemented with the antibiotic required to maintain the antisense construct and are diluted 1:100 in identical media immediately prior to addition to the microtiter plate wells. For a control, cells are also added to several wells that lack antibiotic, but contain the solvent used to dissolve the antibiotics. Cell growth is monitored continuously by incubation at 37° C. in a microtiter plate reader monitoring the OD₆₀₀ of the wells over an 18-hour period. The percent inhibition of growth produced by each concentration of antibiotic is calculated by comparing the rates of logarithmic growth against that exhibited by cells growing in media without antibiotic. A plot of percent inhibition against log[antibiotic concentration] allows extrapolation of an IC₅₀ value for each antibiotic.

E. Measurement of Test Antibiotic Sensitivity in the Presence of Antisense Construct Inducer

The culture media selected for use in the assay is supplemented with inducer at concentrations shown to inhibit cell growth by 50% and 80% as described above, as well as the antibiotic used to maintain the construct. Two fold dilution series of the panel of test antibiotics used above are generated in each of these media. Several antibiotics are tested side by side in each medium with three to four wells being used to evaluate the effects of an antibiotic on cell growth at each concentration. Equal volumes of test antibiotic and cells are added to the wells of a 384 well microtiter plate and mixed. Cells are prepared as described above using the media selected for use in the assay supplemented with the antibiotic required to maintain the antisense construct. The cells are diluted 1:100 into two 50 mL aliquots of identical media containing concentrations of inducer that have been shown to inhibit cell growth by 50% and 80% respectively and incubated at 37° C. with shaking for 2.5 hours. Immediately prior to addition to the microtiter plate wells, the cultures are adjusted to an appropriate OD₆₀₀ (typically 0.002) by dilution into warm (37° C.) sterile media supplemented with identical concentrations of the inducer and antibiotic used to maintain the antisense construct. For a control, cells are also added to several wells that contain solvent used to dissolve test antibiotics but which contain no antibiotic. Cell growth is monitored continuously by incubation at 37° C. in a microtiter plate reader monitoring the OD₆₀₀ of the wells over an 18-hour period. The percent inhibition of growth produced by each concentration of antibiotic is calculated by comparing the rates of logarithmic growth against that exhibited by cells growing in media without antibiotic. A plot of percent inhibition against log[antibiotic concentration] allows extrapolation of an IC₅₀ value for each antibiotic.

F. Determining the Specificity of the Test Antibiotics

A comparison of the IC₅₀s generated by antibiotics of known mechanism of action under antisense induced and non-induced conditions allows the pathway in which a proliferation-required nucleic acid lies to be identified. If cells expressing an antisense nucleic acid complementary to a proliferation-required gene are selectively sensitive to an antibiotic acting via a particular pathway, then the gene against which the antisense acts is involved in the pathway on which the antibiotic acts.

G. Identification of Pathway in Which a Test Antibiotic Acts

As discussed above, the cell-based assay may also be used to determine the pathway against which a test antibiotic acts. In such an analysis, the pathways against which each member of a panel of antisense nucleic acids acts are identified as described above. A panel of cells, each containing an inducible vector which transcribes an antisense nucleic acid complementary to a gene in a known proliferation-required pathway from one of the fusion promoters described herein, is contacted with a test antibiotic for which it is desired to determine the pathway on which it acts under inducing and non-inducing conditions. If heightened sensitivity is observed in induced cells expressing antisense complementary to a gene in a particular pathway but not in induced cells expressing antisense complementary to genes in other pathways, then the test antibiotic acts against the pathway for which heightened sensitivity was observed.

One skilled in the art will appreciate that further optimization of the assay conditions, such as the concentration of inducer used to induce antisense expression and/or the growth conditions used for the assay (for example incubation temperature and media components) may further increase the selectivity and/or magnitude of the antibiotic sensitization exhibited.

The following example confirms the effectiveness of the methods described above.

### EXAMPLE 22

### Identification of the Biological Pathway in Which a Proliferation-Required Gene Lies

The effectiveness of the above assays was validated using proliferation-required genes from E. coli which were identified using procedures similar to those described above. Antibiotics of various chemical classes and modes of action were purchased from Sigma Chemicals (St. Louis, Mo.). Stock solutions were prepared by dissolving each antibiotic in an appropriate aqueous solution based on information provided by the manufacturer. The final working solution of each antibiotic contained no more than 0.2% (w/v) of any organic solvent. To determine their potency against a bacterial strain engineered for expression of an antisense complementary to a proliferation-required 50S ribosomal protein, each antibiotic was serially diluted two or three fold in growth medium supplemented with the appropriate antibiotic for maintenance of the anti-sense construct. At least ten dilutions were prepared for each antibiotic. 25 μL aliquots of each dilution were transferred to discrete wells of a 384-well microplate (the assay plate) using a multi-channel pipette. Quadruplicate wells were used for each dilution of an antibiotic under each treatment condition (plus and minus inducer). Each assay plate contained twenty wells for cell growth controls (growth media replacing antibiotic), ten wells for each treatment (plus and minus inducer, in this example IPTG). Assay plates were usually divided into the two treatments: half the plate containing induced cells and an appropriate concentrations of inducer (in this example IPTG) to maintain the state of induction, the other half containing non-induced cells in the absence of IPTG.

Cells for the assay were prepared as follows. Bacterial cells containing a construct, from which expression of antisense nucleic acid complementary to rplL and rplJ, which encode proliferation-required 50S ribosomal subunit proteins, is inducible in the presence of IPTG, were grown into exponential growth (OD₆₀₀ 0.2 to 0.3) and then diluted 1:100 into fresh media containing either 400 μM or 0 μM inducer (IPTG). These cultures were incubated at 37° C. for 2.5 hr. After a 2.5 hr incubation, induced and non-induced cells were respectively diluted into an assay medium at a final OD₆₀₀ value of 0.0004. The medium contained an appropriate concentration of the antibiotic for the maintenance of the antisense construct. In addition, the medium used to dilute induced cells was supplemented with 800 μM IPTG so that addition to the assay plate would result in a final IPTG concentration of 400 μM. Induced and non-induced cell suspensions were dispensed (25 μl/well) into the appropriate wells of the assay plate as discussed previously. The plate was then loaded into a plate reader, incubated at constant temperature, and cell growth was monitored in each well by the measurement of light scattering at 595 nm. Growth was monitored every 5 minutes until the cell culture attained a stationary growth phase. For each concentration of antibiotic, a percentage inhibition of growth was calculated at the time point corresponding to mid-exponential growth for the associated control wells (no antibiotic, plus or minus IPTG). For each antibiotic and condition (plus or minus IPTG), a plot of percent inhibition versus log of antibiotic concentration was generated and the IC₅₀ determined. A comparison of the IC₅₀ for each antibiotic in the presence and absence of IPTG revealed whether induction of the antisense construct sensitized the cell to the mechanism of action exhibited by the antibiotic. Cells which exhibited a significant (standard statistical analysis) numerical decrease in the IC₅₀ value in the presence of inducer were considered to have an increased sensitivity to the test antibiotic.

The above results demonstrate that induction of an antisense RNA to genes encoding 50S ribosomal subunit proteins results in a selective and highly significant sensitization of cells to antibiotics that inhibit ribosomal function and protein synthesis. The above results further demonstrate that induction of an antisense construct to an essential gene sensitizes a microorganism to compounds that interfere with that gene product's biological role. This sensitization is restricted to compounds that interfere with pathways associated with the targeted gene and its product. It will be appreciated that the cell-based assays described above may be implemented in Staphylococcus aureus and Enterococcus faecalis using proliferation-inhibiting antisense sequences under the control of a fusion promoter described herein.

The example below describes an analysis performed in Staphylococcus aureus.

### EXAMPLE 23

### Identification of the Biological Pathway in Which a Gene Required for Proliferation of Staphylococcus aureus Lies

Sensitized cells were also used to identify the pathway in which a gene required for proliferation of Staphylococcus aureus lies. Antibiotics of various chemical classes and modes of action were purchased from chemical suppliers, for example Sigma Chemicals (St. Louis, Mo.). Stock solutions were prepared by dissolving each antibiotic in an appropriate aqueous solution based on information provided by the manufacturer. The final working solution of each antibiotic contained no more than 0.2% (w/v) of any organic solvent.

To determine its potency against a bacterial strain containing an antisense nucleic acid complementary to the sequence encoding the Beta (>) subunit of gyrase (which is required for proliferation) under the control of the Xyl-T5 fusion promoter, each antibiotic was serially diluted two or three fold in growth medium supplemented with the appropriate antibiotic for maintenance of the anti-sense construct. At least ten dilutions were prepared for each antibiotic.

Aliquots (25 μL) of each dilution were transferred to discrete wells of a 384-well microplate (the assay plate) using a multi-channel pipette. Quadruplicate wells were used for each dilution of an antibiotic under each treatment condition (plus and minus inducer). Each assay plate retained twenty wells for cell growth controls (growth media replacing antibiotic), ten wells for each treatment (plus and minus inducer, in this example xylose). Half the assay plate contained induced cells (in this example Staphylococcus aureus cells) and appropriate concentrations of inducer (in this example xylose) to maintain the state of induction while the other half of the assay plate contained non-induced cells in the absence of inducer.

Cells of a bacterial clone containing a construct in which expression of antisense complementary to the sequence encoding the β subunit of gyrase under the control of a xylose inducible promoter were grown into exponential growth (OD₆₀₀ 0.2 to 0.3) and then diluted 1:100 into fresh media containing either 12 mM or 0 mM inducer (xylose). These cultures were incubated at 37° C. for 2.5 hr. The presence of inducer (xylose) in the medium initiates and maintains production of antisense RNA from the antisense construct, in this case. After a 2.5 hr incubation, induced and non-induced cells were respectively diluted into an assay medium containing an appropriate concentration of the antibiotic for the maintenance of the anti-sense construct. In addition, medium used to dilute induced cells was supplemented with 24 mM xylose so that addition to the assay plate would result in a final xylose concentration of 12 mM. The cells were diluted to a final OD₆₀₀ value of 0.0004.

Induced and non-induced cell suspensions were dispensed (25 :l/well) into the appropriate wells of the assay plate as discussed previously. The plate was then loaded into a plate reader and incubated at constant temperature while cell growth was monitored in each well by the measurement of light scattering at 595 nm. Growth was monitored every 5 minutes until the cell culture attained a stationary growth phase. For each concentration of antibiotic, a percentage inhibition of growth was calculated at the time point corresponding to mid-exponential growth for the associated control wells (no antibiotic, plus or minus xylose). For each antibiotic and condition (plus or minus xylose), plots of percent inhibition versus Log of antibiotic concentration were generated and IC₅₀ₛ determined.

A comparison of each antibiotic's IC₅₀ in the presence and absence of inducer (in this example xylose) reveals whether induction of the antisense construct sensitized the cell to the antibiotic's mechanism of action. If the antibiotic acts against the P subunit of gyrase, the IC₅₀ of induced cells will be significantly lower than the IC₅₀ of uninduced cells.

FIG. 15 lists the antibiotics tested, their targets, and their fold increase in potency between induced cells and uninduced cells. As illustrated in FIG. 15, the potency of cefotaxime, cefoxitin, fusidic acid, lincomycin, tobramycin, trimethoprim and vancomycin, each of which act on targets other than the β subunit of gyrase, was not significantly different in induced cells as compared to uninduced cells. However, the potency of novobiocin, which acts against the β subunit of gyrase, was significantly different between induced cells and uninduced cells.

Thus, induction of an antisense nucleic acid complementary to the sequence encoding the β subunit of gyrase results in a selective and significant sensitization of Staphylococcus aureus cells to an antibiotic which inhibits the activity of this protein. Furthermore, the results demonstrate that induction of an antisense construct to an essential gene sensitizes a microorganism to compounds that interfere with that gene product's biological role. This sensitization is apparently restricted to compounds that interfere with the targeted gene and its product. It will be appreciated that the cell-based assays described above may be implemented in Enterococcus faecalis using proliferation-inhibiting antisense sequences under the control of a fusion promoter described herein. It will also be appreciated that the above cell-based assays employing the fusion promoters described herein can be implemented in other Gram-positive organims, including but not limited to, Bacillus anthracis, Clostridium botulinum, Clostridium difficile, Clostridium perfringens, Clostridium teteni, Corynebacterium diptheriae, Enterococcus faecalis, Enterococcus faecium, Lactococcus lactis, Listeria monocytogenes, Mycobacterium leprae, Mycobacterium tuberculosis, Nocardia asteroides, Staphylococcus aureus, Staphylococcus epidermidis, Staphylococcus xylosis, Streptococcus pneumoniae, Streptococcus mutans or any species falling within the genera of any of the above species.

Assays utilizing antisense constructs to essential genes can be used to identify compounds that interfere with the activity of those gene products. Such assays could be used to identify drug leads, for example antibiotics.

Assays utilizing antisense constructs to essential genes can be used to identify compounds that specifically interfere with the activity of multiple targets in a pathway. Such constructs can be used to simultaneously screen a sample against multiple targets in one pathway in one reaction (Combinatorial HTS).

Furthermore, as discussed above, panels of antisense construct-containing cells may be used to characterize the point of intervention of any compound affecting an essential biological pathway including antibiotics with no known mechanism of action.

The following example provides the a method for determining the pathway on which an antibiotic acts.

### EXAMPLE 24

### Cell-Based Assays Using Promoter Replacement or Operator Insertion

A gene encoding a gene product required for cellular proliferation is placed under the control of an inducible promoter, such as one of the inducible fusion promoters described herein, using the promoter replacement or operator insertion methods described above. Sensitized cells are generated by growing cells in which the gene encoding the gene product required for proliferation is under control of the inducible fusion promoter in medium containing varying concentrations of inducer which are less than the inducer concentration in which the promoter has maximal activity. The sensitized cells are contacted with candidate compounds to be tested for the ability to inhibit proliferation. In some embodiments, the candidate compounds may be compounds in a library of compounds generated using combinatorial chemistry techniques or compounds in a natural products library. The extent of proliferation of the sensitized cells is compared to the extent of proliferation of control cells in which the activity or level of the gene product is higher than in the sensitized cells. For example, the control cells may be cells in which the inducible fusion promoter is fully active. A compound which inhibits the growth of the sensitized cells to a substantially greater extent than the control cells is identified. This compound may be used as a drug or further optimized to generate a more potent compound.

It will be appreciated that the cell-based assays described above may be implemented in Staphylococcus aureus and Enterococcus faecalis using the fusion promoters or operators described herein. It will also be appreciated that the above cell-based assays employing the fusion promoters or operators described herein can be implemented in other Gram-positive organims, including but not limited to, Bacillus anthracis, Clostridium botulinum, Clostridium difficile, Clostridium perfringens, Clostridium teteni, Corynebacterium diptheriae, Enterococcus faecalis, Enterococcus faecium, Lactococcus lactis, Listeria monocytogenes, Mycobacterium leprae, Mycobacterium tuberculosis, Nocardia asteroides, Staphylococcus aureus, Staphylococcus epidermidis, Staphylococcus xylosis, Streptococcus pneumoniae, Streptococcus mutans or any species falling within the genera of any of the above species.

The example below describes an approach to determine if an antisense nucleic acid known to inhibit cell proliferation of one organism can successfully inhibit cell proliferation of a second organism.

### EXAMPLE 25

### Identification of Proliferation-Required Genes in Using Antisense Nucleic Acids from Heterologous Organisms

Once an antisense nucleic acid which inhibits the proliferation of a Gram-positive organism has been identified using the fusion promoters described herein, the antisense nucleic acid may be used to identify proliferation-required genes in heterologous organisms. In such methods, the antisense nucleic acid is transcribed in the heterologous organism and its ability to inhibit proliferation of the heterologous organism is determined.

In such embodiments, the antisense nucleic acids are inserted into expression vectors functional in the organisms in which the antisense nucleic acids are evaluated. Those skilled in the art will appreciate that a negative result in a heterologous microorganism does not mean that that microorganism is missing that gene nor does it mean that the gene is unessential. However, a positive result means that the heterologous microorganism contains a homologous gene which is required for proliferation of that microorganism. The homologous gene may be obtained using the methods described herein. Those cells that are inhibited by antisense may be used in cell-based assays as described herein for the identification and characterization of compounds in order to develop antibiotics effective in these microorganisms. Those skilled in the art will appreciate that an antisense molecule that works in the microorganism from which it was obtained will not always work in a heterologous microorganism.

The antisense nucleic acids that inhibit the growth of Staphylococcus aureus or Enterococcus faecalis, for example, may also be evaluated for their ability to inhibit the growth of microorganisms other than Staphylococcus aureus or Enterococcus faecalis. In particular, the ability of the antisense nucleic acid to inhibit the growth of Aspergillus fumigatus, Bacillus anthracis, Burkholderia cepacia, Campylobacter jejuni, Candida albicans, Chlamydia pneumoniae, Chlamydia trachomatus, Clostridium botulinum, Cryptococcus neoformans, Enterobacter cloacae, Enterococcusfaecalis, Escherichia coli, Haemophilus influenzae, Helicobacter pylori, Klebsiella pneumoniae, Mycobacterium leprae, Mycobacterium tuberculosis, Neisseria gonorrhoeae, Nocardia asteroides, Pseudomonas aeruginosa, Salmonella cholerasuis, Salmonella paratyphi, Salmonella typhi, Salmonella typhimurium, Staphylococcus aureus, Klebsiella pneumoniae, Moxarella catarrhalis, Shigella boydii, Shigella dysenteriae, Shigella flexneri, Shigella sonnet, Pseudomonas aeruginosa, Staphylococcus epidennidis, Streptococcus pneumoniae, Treponema pallidum, Yersinia pestis, or any species falling within the genera of any of the above species may be evaluated.

In such methods, expression vectors in which the expression of an antisense nucleic acid that inhibits the growth of Staphylococcus aureus or Enterococcus faecalis is under the control of an inducible promoter, such as a fusion promoter described herein, are introduced into the microorganisms in which they are to be evaluated. In some embodiments, the antisense nucleic acids may be evaluated in microorganisms which are closely related to Staphylococcus aureus or Enterococcus faecalis. The ability of these antisense nucleic acids to inhibit the growth of the related microorganisms in the presence of the inducer is then measured.

In one example, thirty-nine antisense nucleic acids which inhibited the growth of Staphylococcus aureus were identified using methods such as those described herein and were inserted into an expression vector such that their expression was under the control of a xylose-inducible Xyl-T5 promoter. A vector with a reporter gene under control of the Xyl-T5 promoter was used to show that expression from the Xyl-T5 promoter in Staphylococcus epidermidis was comparable to that in Staphylococcus aureus.

The vectors were introduced into Staphylococcus epidermidis by electroporation as follows: Staphylococcus epidermidis was grown in liquid culture to mid-log phase and then harvested by centrifugation. The cell pellet was resuspended in 1/3 culture volume of ice-cold EP buffer (0.625 M sucrose, 1 mM MgCl₂, pH=4.0), and then harvested again by centrifugation. The cell pellet was then resuspended with 1/40 volume EP buffer and allowed to incubate on ice for 1 hour. The cells were then frozen for storage at −80° C. For electroporation, 50 μl of thawed electrocompetent cells were combined with 0.5 μg plasmid DNA and then subjected to an electrical pulse of 10 kV/cm, 25 uFarads, 200 ohm using a BioRad gene pulser electroporation device. The cells were immediately resuspended with 200 μl outgrowth medium and incubated for 2 hours prior to plating on solid growth medium with drug selection to maintain the plasmid vector. Colonies resulting from overnight growth of these platings were selected, cultured in liquid medium with drug selection, and then subjected to dilution plating analysis as described above to test growth sensitivity in the presence of the inducer xylose.

The results are shown in Table I below. The first column indicates the Molecule Number of the Staphylococcus aureus antisense nucleic acid which was introduced into Staphylococcus epidermidis. The second column indicates whether the antisense nucleic acid inhibited the growth of Staphylococcus epidermidis, with a “+” indicating that growth was inhibited. Of the 39 Staphylococcus aureus antisense nucleic acids evaluated, 20 inhibited the growth of Staphylococcus epidermidis.

TABLE I
Sensitivity of Other Microorganisms to Antisense Nucleic Acids That
Inhibit Proliferation of Staphylococcus aureus

| Mol. No. | S. epidermidis |
|---|---|
| SaXA005 | + |
| SaXA007 | + |
| SaXA008 | + |
| SaXA009 | + |
| SaXA010 | + |
| SaXA011 | − |
| SaXA012 | − |
| SaXA013 | − |
| SaXA015 | + |
| SaXA017 | − |
| SaXA022 | + |
| SaXA023 | − |
| SaXA024 | − |
| SaXA025 | + |
| SaXA026 | + |
| SaXA027 | − |
| SaXA027b | − |
| SaXA02c | − |
| SaXA028 | − |
| SaXA029 | + |
| SaXA030 | + |
| SaXA032 | + |
| SaXA033 | + |
| SaXA034 | − |
| SaXA035 | + |
| SaXA037 | + |
| SaXA039 | − |
| SaXA042 | − |
| SaXA043 | − |
| SaXA044 | − |
| SaXA045 | + |
| SaXA051 | + |
| SaXA053 | − |
| SaXA056b | − |
| SaXA059a | + |
| SaXA060 | − |
| SaXA061 | + |
| SaXA062 | + |
| SaXA063 | − |
| SaXA065 | − |

It will be appreciated above methods for identifying heterologous proliferation-required genes may be implemented in Enterococcus faecalis using the fusion promoters or operators described herein. It will be appreciated that the above methods for identifying heterologous proliferation-required genes using the fusion promoters described herein can be implemented in other Gram-positive organims, including but not limited to, Bacillus anthracis, Clostridium botulinum, Clostridium difficile, Clostridium perfringens, Clostridium teteni, Corynebacterium diptheriae, Enterococcus faecalis, Enterococcus faecium, Lactococcus lactis, Listeria monocytogenes, Mycobacterium leprae, Mycobacterium tuberculosis, Nocardia asteroides, Staphylococcus aureus, Staphylococcus epidermidis, Staphylococcus xylosis, Streptococcus pneumoniae, Streptococcus mutans or any species falling within the genera of any of the above species.

Those of skill in the art will appreciate that the methods disclosed herein provide a relatively easy, rapid, and economical way of identifying essential genes in an organism and identifying compounds that alter the expression levels of the identified essential genes.

One skilled in the art will appreciate that these methods and devices are and may be adapted to carry out the objects and obtain the ends and advantages mentioned, as well as those inherent therein. The methods, procedures, and devices described herein are presently representative of preferred embodiments and are exemplary and are not intended as limitations on the scope of the invention. Changes therein and other uses will occur to those skilled in the art which are encompassed within the spirit of the invention and are defined by the scope of the disclosure.

It will be apparent to one skilled in the art that varying substitutions and modifications may be made to the invention disclosed herein without departing from the scope and spirit of the invention.

Those skilled in the art recognize that the aspects and embodiments of the invention set forth herein may be practiced separate from each other or in conjunction with each other. Therefore, combinations of separate embodiments are within the scope of the invention as disclosed herein.

All patents and publications mentioned in the specification are indicative of the levels of those skilled in the art to which the invention pertains. All patents and publications are herein incorporated by reference to the same extent as if each individual publication was specifically and individually indicated to be incorporated by reference.

The invention illustratively described herein suitably may be practiced in the absence of any element or elements, limitation or limitations which is not specifically disclosed herein. Thus, for example, in each instance herein any of the terms “comprising”, “consisting essentially of” and “consisting of” may be replaced with either of the other two terms. The terms and expressions which have been employed are used as terms of description and not of limitation, and there is no intention that in the use of such terms and expressions indicates the exclusion of equivalents of the features shown and described or portions thereof. It is recognized that various modifications are possible within the scope of the invention disclosed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the disclosure.

In addition, where features or aspects of the invention are described in terms of Markush groups, those skilled in the art will recognize that the invention is also thereby described in terms of any individual member or subgroup of members of the Markush group.

What is claimed is:

1. An isolated nucleic acid comprising a fusion promoter said fusion promoter comprising at least one promoter that is modified to have altered activity in at least one gram-positive organism said promoter being linked to at least one operator selected from the group consisting of xylO, tetO, trpO, malO and λc1O, wherein said at least one operator is positioned such that binding of at least one repressor to said at least one operator represses transcription from said fusion promoter.

2. The isolated fusion promoter of claim 1, wherein said at least one promoter is selected from the group consisting of SEQ ID NOs.: 36-45.

3. The isolated nucleic acid of claim 1, wherein said at least one operator is xylO.

4. The isolated nucleic acid of claim 3, wherein said at least one promoter is T5.

5. The isolated nucleic acid of claim 3, further comprising a second operator.

6. The isolated nucleic acid of claim 5, wherein said second operator is lacO.

7. The isolated nucleic acid of claim 1, wherein said fusion promoter is responsive to an inducer.

8. The isolated nucleic acid of claim 7, wherein said inducer is xylose.

9. The isolated nucleic acid of claim 1, wherein said fusion promoter is titratable.

10. The isolated nucleic acid of claim 1, wherein said at least one gram-positive organism is selected from the group consisting of Bacillus anthracis, Clostridium botulinum, Clostridium difficile, Clostridium perfringens, Clostridium teteni, Corynebacterium diptheriae, Enterococcus faecalis, Enterococcus faecium, Lactococcus lactis, Listeria monocytogenes, Mycobacterium leprae, Mycobacterium tuberculosis, Nocardia asteroides, Staphylococcus aureus, Staphylococcus epidermidis, Staphylococcus xylosis, Streptococcus pneumoniae, Streptococcus mutans and any species falling within the genera of any of the above species.

11. The isolated nucleic acid of claim 1, wherein said at least one gram-positive organism is Staphylococcus aureus.

12. The isolated nucleic acid of claim 1, wherein said at least one gram-positive organism is Enterococcus faecalis.

13. An isolated nucleic acid comprising a fusion promoter said fusion promoter comprising at least one promoter selected from the group consisting of T5, CP25, P32, P59, P1P2 and PL, said promoter being linked to at least one operator selected from the group consisting of xylO, tetO, trpO, malO and λc1O, wherein said at least one operator is positioned such that binding of at least one repressor to said at least one operator represses transcription from said fusion promoter.

14. The isolated nucleic acid of claim 13, wherein said at least one operator is xylO.

15. The isolated nucleic acid of claim 14, wherein said at least one promoter is T5.

16. The isolated nucleic acid of claim 14, further comprising a second operator.

17. The isolated nucleic acid of claim 16, wherein said second operator is lacO.

18. The isolated nucleic acid of claim 13, wherein said fusion promoter is responsive to an inducer.

19. The isolated nucleic acid of claim 18, wherein said inducer is xylose.

20. The isolated nucleic acid of claim 13, wherein said fusion promoter is titratable.

21. The isolated nucleic acid of claim 13, wherein said at least one gram-positive organism is selected from the group consisting of Bacillus anthracis, Clostridium botulinum, Clostridium difficile, Clostridium perfringens, Clostridium teteni, Corynebacterium diptheriae, Enterococcus faecalis, Enterococcus faecium, Lactococcus lactis, Listeria monocytogenes, Mycobacterium leprae, Mycobacterium tuberculosis, Nocardia asteroides, Staphylococcus aureus, Staphylococcus epidermidis, Staphylococcus xylosis, Streptococcus pneumoniae, Streptococcus mutans and any species falling within the genera of any of the above species.

22. The isolated nucleic acid of claim 13, wherein said at least one gram-positive organism is Staphylococcus aureus.

23. The isolated nucleic acid of claim 13, wherein said at least one gram-positive organism is Enterococcus faecalis.

24. An isolated fusion promoter comprising one of SEQ ID NO.: 26-35.

25. A vector comprising the isolated nucleic acid of claim 1.

26. A vector comprising the isolated nucleic acid of claim 13.

27. A vector comprising the isolated nucleic acid of claim 24.

28. The vector of claim 25 further comprising at least one replicon selected from the group consisting of p15a, pC194 and pCT1138.

29. The vector of claim 25 further comprising a reporter gene operably linked to said fusion promoter.

30. The vector of claim 29, wherein said reporter gene is lacL-lacM.

31. The vector of claim 30, wherein lacL-lacM is derived from Leuconostoc mesenteroides.

32. The vector of claim 25, wherein said at least one operator is xylO.

33. The vector of claim 32, wherein said at least one promoter is T5.

34. The vector of claim 32, further comprising a second operator.

35. The vector of claim 34, wherein said second operator is lacO.

36. The vector of claim 25, wherein said fusion promoter is responsive to an inducer.

37. The vector of claim 36, wherein said inducer is xylose.

38. The vector of claim 25, wherein said fusion promoter is titratable.

39. The vector of claim 25, wherein said at least one gram-positive organism is selected from the group consisting of Bacillus anthracis, Clostridium botulinum, Clostridium difficile, Clostridium perfringens, Clostridium teteni, Corynebacterium diptheriae, Enterococcus faecalis, Enterococcus faecium, Lactococcus lactis, Listeria monocytogenes, Mycobacterium leprae, Mycobacterium tuberculosis, Nocardia asteroides, Staphylococcus aureus, Staphylococcus epidermidis, Staphylococcus xylosis, Streptococcus pneumoniae, Streptococcus mutans and any species falling within the genera of any of the above species.

40. The vector of claim 25, wherein said at least one gram-positive organism is Staphylococcus aureus.

41. The vector of claim 25, wherein said at least one gram-positive organism is Enterococcus faecalis.

42. The vector of claim 25, further comprising a random fragment of a microbial genome operably linked to said fusion promoter.

43. The vector of claim 25, further comprising a nucleic acid that encodes a peptide, wherein said nucleic acid is operably linked to said fusion promoter.

44. The vector of claim 25, further comprising a nucleic acid that is complementary to a portion of a microbial genome, wherein said nucleic acid is operably linked to said fusion promoter.

45. The vector of claim 25, further comprising a nucleic acid that encodes a molecule that inhibits the proliferation of microbe, wherein said nucleic acid is operably linked to said fusion promoter.

46. A host cell comprising the nucleic acid of claim 1.

47. A host cell comprising the nucleic acid of claim 13

48. A method for identifying genes involved in cellular proliferation said method comprising the steps of:
(a) introducing into cells of a cell population a construct comprising an inducible fusion promoter operably linked to a nucleic acid, said fusion promoter comprising at least one promoter that is modified to have altered activity in at least one gram-positive organism said promoter being linked to at least one operator selected from the group consisting of xylO, tetO, trpO, malO and λc1O, wherein said at least one operator is positioned such that binding of at least one repressor to said at least one operator represses transcription from said fusion promoter;
(b) inducing transcription of said nucleic acid from said inducible fusion promoter;
(c) identifying the cells in said cell population whose proliferation is reduced in response to the induction of transcription of said nucleic acid; and
(d) identifying the gene from a cell identified in step (c) to which at least a portion of said nucleic acid is complementary.

49. A method for identifying genes involved in cellular proliferation said method comprising the steps of:
(a) introducing into cells of a cell population a construct comprising an inducible fusion promoter operably linked to a nucleic acid, said fusion promoter comprising at least one promoter selected from the group consisting of T5, CP25, P32, P59, P1P2 and PL, said promoter being linked to at least one operator selected from the group consisting of xylO, tetO, trpO, malO and λc1O, wherein said at least one operator is positioned such that binding of at least one repressor to said at least one operator represses transcription from said fusion promoter;
(b) inducing transcription of said nucleic acid from said inducible fusion promoter;
(c) identifying the cells in said cell population whose proliferation is reduced in response to the induction of transcription of said nucleic acid; and
(d) identifying the gene from a cell identified in step (c) to which at least a portion of said nucleic acid is complementary.

50. The method of claim 48, wherein said nucleic acid is random fragment of a microbial genome.

51. The method of claim 48, wherein said nucleic acid encodes an aptamer.

52. The method of claim 48, wherein said nucleic acid encodes a transcript that is complementary to a portion of a microbial genome.

53. The method of claim 48, wherein said nucleic acid encodes a molecule that inhibits the proliferation of a microbe.

54. The method of claim 48, wherein said at least one operator is xylO.

55. The method of claim 54, wherein said fusion promoter is T5.

56. The method of claim 54, wherein said fusion promoter further comprises a second operator.

57. The method of claim 56, wherein said second operator is lacO.

58. The method of claim 54, wherein said inducer is xylose.

59. The method of claim 54, wherein said fusion promoter is titratable.

60. The method of claim 48, wherein said at least one gram-positive organism is selected from the group consisting of Bacillus anthracis, Clostridium botulinum, Clostridium difficile, Clostridium perfringens, Clostridium teteni, Corynebacterium diptheriae, Enterococcus faecalis, Enterococcus faecium, Lactococcus lactis, Listeria monocytogenes, Mycobacterium leprae, Mycobacterium tuberculosis, Nocardia asteroides, Staphylococcus aureus, Staphylococcus epidermidis, Staphylococcus xylosis, Streptococcus pneumoniae, Streptococcus mutans and any species falling within the genera of any of the above species.

61. The method of claim 48, wherein said at least one gram-positive organism is Staphylococcus aureus.

62. The method of claim 48, wherein said at least one gram-positive organism is Enterococcus faecalis.

63. A method for identifying genes involved in cellular proliferation said method comprising the steps of:
(a) introducing into the genome of a cell a construct comprising an inducible fusion promoter operably linked to a nucleic acid, said fusion promoter comprising at least one promoter that is modified to have altered activity in at least one gram-positive organism said promoter being linked to at least one operator selected from the group consisting of xylO, tetO, trpO, malO and λc1O, wherein said at least one operator is positioned such that binding of at least one repressor to said at least one operator represses transcription from said fusion promoter;
(b) comparing the proliferation of said cell cultured in the presence of a first concentration of an inducer that induces transcription from said fusion promoter with the proliferation of said cell cultured in the presence of a concentration of said inducer that is less than said first concentration, wherein a difference in proliferation indicates that said fusion promoter modulates a gene that is required for proliferation; and
(c) identifying the gene that is modulated by said fusion promoter.

64. A method for identifying genes involved in cellular proliferation said method comprising the steps of:
(a) introducing into the genome of a cell a construct comprising an inducible fusion promoter operably linked to a nucleic acid, said fusion promoter comprising at least one promoter selected from the group consisting of T5, CP25, P32, P59, P1P2 and PL, said promoter being linked to at least one operator selected from the group consisting of xylO, tetO, trpO, malO and λc1O, wherein said at least one operator is positioned such that binding of at least one repressor to said at least one operator represses transcription from said fusion promoter;
(b) comparing the proliferation of said cell cultured in the presence of a first concentration of an inducer that induces transcription from said fusion promoter with the proliferation of said cell cultured in the presence of a concentration of said inducer that is less than said first concentration, wherein a difference in proliferation indicates that said fusion promoter modulates a gene that is required for proliferation; and
(c) identifying the gene that is modulated by said fusion promoter.

65. The method of claim 63, wherein said nucleic acid is random fragment of a microbial genome.

66. The method of claim 63, wherein said nucleic acid encodes an aptamer.

67. The method of claim 63, wherein said nucleic acid encodes a transcript that is complementary to a portion of a microbial genome.

68. The method of claim 63, wherein said nucleic acid encodes a molecule that inhibits the proliferation of a microbe.

69. The method of claim 63, wherein said at least one operator is xylO.

70. The method of claim 69, wherein said at least one promoter is T5.

71. The method of claim 69, wherein said fusion promoter further comprises a second operator.

72. The method of claim 71 wherein said second operator is lacO.

73. The method of claim 63, wherein said inducer is xylose.

74. The method of claim 63, wherein said fusion promoter is titratable.

75. The method of claim 63, wherein said at least one gram-positive organism is selected from the group consisting of Bacillus anthracis, Clostridium botulinum, Clostridium difficile, Clostridium perfringens, Clostridium teteni, Corynebacterium diptheriae, Enterococcus faecalis, Enterococcus faecium, Lactococcus lactis, Listeria monocytogenes, Mycobacterium leprae, Mycobacterium tuberculosis, Nocardia asteroides, Staphylococcus aureus, Staphylococcus epidermidis, Staphylococcus xylosis, Streptococcus pneumoniae, Streptococcus mutans and any species falling within the genera of any of the above species.

76. The method of claim 63, wherein said at least one gram-positive organism is Staphylococcus aureus.

77. The method of claim 63, wherein said at least one gram-positive organism is Enterococcus faecalis.

78. A method for identifying a compound which reduces the activity or level of a gene product required for proliferation of a cell said method comprising:
(a) introducing into a cell a construct comprising an inducible fusion promoter operably linked to a nucleic acid that is complementary to at least a portion of a proliferation-required gene, said fusion promoter comprising at least one promoter that is modified to have altered activity in at least one gram-positive organism said promoter being linked to at least one operator selected from the group consisting of xylO, tetO, trpO, malO and λc1O, wherein said at least one operator is positioned such that binding of at least one repressor to said at least one operator represses transcription from said fusion promoter;
(b) sensitizing said cell by inducing transcription from said fusion promoter;
(c) contacting said sensitized cell with a compound; and
(d) determining the degree to which said compound inhibits proliferation of said sensitized cell relative to a cell which has not been sensitized.

79. A method for identifying a compound which reduces the activity or level of a gene product required for proliferation of a cell said method comprising:
(a) introducing into a cell a construct comprising an inducible fusion promoter operably linked to a nucleic acid that is complementary to at least a portion of a proliferation-required gene, said fusion promoter comprising at least one promoter selected from the group consisting of T5, CP25, P32, P59, P1P2 and PL, said promoter being linked to at least one operator selected from the group consisting of xylO, tetO, trpO, malO and λc1O, wherein said at least one operator is positioned such that binding of at least one repressor to said at least one operator represses transcription from said fusion promoter;
(b) sensitizing said cell by inducing transcription from said fusion promoter;
(c) contacting said sensitized cell with a compound; and
(d) determining the degree to which said compound inhibits proliferation of said sensitized cell relative to a cell which has not been sensitized.

80. The method of claim 78, wherein said at least one operator is xylO.

81. The method of claim 80, wherein said at least one promoter is T5.

82. The method of claim 80, wherein said fusion promoter further comprises a second operator.

83. The method of claim 82, wherein said second operator is lacO.

84. The method of claim 78, wherein said inducer is xylose.

85. The method of claim 78, wherein said fusion promoter is titratable.

86. The method of claim 78, wherein said at least one gram-positive organism is selected from the group consisting of Bacillus anthracis, Clostridium botulinum, Clostridium difficile, Clostridium perfringens, Clostridium teteni, Corynebacterium diptheriae, Enterococcus faecalis, Enterococcus faecium, Lactococcus lactis, Listeria monocytogenes, Mycobacterium leprae, Mycobacterium tuberculosis, Nocardia asteroides, Staphylococcus aureus, Staphylococcus epidermidis, Staphylococcus xylosis, Streptococcus pneumoniae, Streptococcus mutans and any species falling within the genera of any of the above species.

87. The method of claim 78, wherein said at least one gram-positive organism is Staphylococcus aureus.

88. The method of claim 78, wherein said at least one gram-positive organism is Enterococcus faecalis.

89. A compound identified using the method of claim 78.

90. A method for inhibiting the activity or expression of a gene in an operon required for proliferation said method comprising:
(a) introducing into a cell a construct comprising an inducible fusion promoter operably linked to a nucleic acid that is complementary to at least a portion of a proliferation-required operon, said fusion promoter comprising at least one promoter that is modified to have altered activity in at least one gram-positive organism said promoter being linked to at least one operator selected from the group consisting of xylO, tetO, trpO, malO and λc1O, wherein said at least one operator is positioned such that binding of at least one repressor to said at least one operator represses transcription from said fusion promoter; and
(b) inducing transcription from said fusion promoter.

91. A method for inhibiting the activity or expression of a gene in an operon required for proliferation said method comprising:
(a) introducing into a cell a construct comprising an inducible fusion promoter operably linked to a nucleic acid that is complementary to at least a portion of a proliferation-required operon, said fusion promoter comprising at least one promoter selected from the group consisting of T5, CP25, P32, P59, P1P2 and PL, said promoter being linked to at least one operator selected from the group consisting of xylO, tetO, trpO, malO and λc1O, wherein said at least one operator is positioned such that binding of at least one repressor to said at least one operator represses transcription from said fusion promoter; and
(b) inducing transcription from said fusion promoter.

92. The method of claim 90, wherein said at least one operator is xylO.

93. The method of claim 92, wherein said at least one promoter is T5.

94. The method of claim 92, wherein said fusion promoter further comprises a second operator.

95. The method of claim 94, wherein said second operator is lacO.

96. The method of claim 90, wherein said inducer is xylose.

97. The method of claim 90, wherein said fusion promoter is titratable.

98. The method of claim 90, wherein said at least one gram-positive organism is selected from the group consisting of Bacillus anthracis, Clostridium botulinum, Clostridium difficile, Clostridium perfringens, Clostridium teteni, Corynebacterium diptheriae, Enterococcus faecalis, Enterococcus faecium, Lactococcus lactis, Listeria monocytogenes, Mycobacterium leprae, Mycobacterium tuberculosis, Nocardia asteroides, Staphylococcus aureus, Staphylococcus epidermidis, Staphylococcus xylosis, Streptococcus pneumoniae, Streptococcus mutans and any species falling within the genera of any of the above species.

99. The method of claim 90, wherein said at least one gram-positive organism is Staphylococcus aureus.

100. The method of claim 90, wherein said at least one gram-positive organism is Enterococcus faecalis.

101. A method of manufacturing an antibiotic comprising the steps of:
(a) introducing into a cell a construct comprising an inducible fusion promoter operably linked to a nucleic acid that is complementary to at least a portion of a proliferation-required gene, said fusion promoter comprising at least one promoter that is modified to have altered activity in at least one gram-positive organism said promoter being linked to at least one operator selected from the group consisting of xylO, tetO, trpO, malO and λc1O, wherein said at least one operator is positioned such that binding of at least one repressor to said at least one operator represses transcription from said fusion promoter;
(b) sensitizing said cell by inducing transcription from said fusion promoter;
(c) contacting said sensitized cell with a compound;
(d) identifying a compound which substantially inhibits the proliferation of said sensitized cell relative to a cell which has not been sensitized; and
(e) manufacturing the compound so identified.

102. A method of manufacturing an antibiotic comprising the steps of:
(a) introducing into a cell a construct comprising an inducible fusion promoter operably linked to a nucleic acid that is complementary to at least a portion of a proliferation-required gene, said fusion promoter comprising at least one promoter selected from the group consisting of T5, CP25, P32, P59, P1P2 and PL, said promoter being linked to at least one operator selected from the group consisting of xylO, tetO, trpO, malO and λc1O, wherein said at least one operator is positioned such that binding of at least one repressor to said at least one operator represses transcription from said fusion promoter;
(b) sensitizing said cell by inducing transcription from said fusion promoter;
(c) contacting said sensitized cell with a compound;
(d) identifying a compound which substantially inhibits the proliferation of said sensitized cell relative to a cell which has not been sensitized; and
(e) manufacturing the compound so identified.

103. The method of claim 101, wherein said at least one operator is xylO.

104. The method of claim 103, wherein said at least one promoter is T5.

105. The method of claim 103, wherein said fusion promoter further comprises a second operator.

106. The method of claim 105, wherein said second operator is lacO.

107. The method of claim 101, wherein said inducer is xylose.

108. The method of claim 101, wherein said fusion promoter is titratable.

109. The method of claim 101, wherein said at least one gram-positive organism is selected from the group consisting of Bacillus anthracis, Clostridium botulinum, Clostridium difficile, Clostridium perfringens, Clostridium teteni, Corynebacterium diptheriae, Enterococcus faecalis, Enterococcus faecium, Lactococcus lactis, Listeria monocytogenes, Mycobacterium leprae, Mycobacterium tuberculosis, Nocardia asteroides, Staphylococcus aureus, Staphylococcus epidermidis, Staphylococcus xylosis, Streptococcus pneumoniae, Streptococcus mutans and any species falling within the genera of any of the above species.

110. The method of claim 101, wherein said at least one gram-positive organism is Staphylococcus aureus.

111. The method of claim 101, wherein said at least one gram-positive organism is Enterococcusfaecalis.

112. A method for identifying a gene which is required for proliferation of a prokaryotic cell said method comprising the steps of:
(a) replacing the native promoter of a gene in the chromosome of a prokaryotic cell having an enhanced frequency of homologous recombination with a regulatable fusion promoter comprising at least one promoter that is modified to have altered activity in at least one gram-positive organism said promoter being linked to at least one operator selected from the group consisting of xylO, tetO, trpO, malO, lacO and λc1O, wherein said at least one operator is positioned such that binding of at least one repressor to said at least one operator represses transcription from said fusion promoter; and
(b) identifying cells in which the extent of proliferation of said cell when said fusion promoter is active at a first level is substantially different than the extent of proliferation of said cell when said fusion promoter is active at a second level, said first level being greater than said second level.

113. A method for identifying a gene which is required for proliferation of a prokaryotic cell said method comprising the steps of:
(a) replacing the native promoter of a gene in the chromosome of a prokaryotic cell having an enhanced frequency of homologous recombination with a regulatable fusion promoter comprising at least one promoter selected from the group consisting of T5, CP25, P32, P59, P1P2 and PL, said promoter being linked to at least one operator selected from the group consisting of xylO, tetO, trpO, malO, lacO and λc1O, wherein said at least one operator is positioned such that binding of at least one repressor to said at least one operator represses transcription from said fusion promoter; and
(b) identifying cells in which the extent of proliferation of said cell when said fusion promoter is active at a first level is substantially different than the extent of proliferation of said cell when said fusion promoter is active at a second level, said first level being greater than said second level.

114. The method of claim 112, wherein said fusion promoter is an inducible promoter.

115. The method of claim 112, wherein the step of replacing said native promoter comprises introducing a linear nucleic acid comprising a 5′ portion homologous to a first portion of said native promoter, a 3′ portion homologous to a second portion of said native promoter and said fusion promoter disposed between said 5′ portion and said 3′ portion into said cell such that homologous recombination occurs between said 5′ portion and said first portion of said native promoter and between said 3′ portion and said second portion of said native promoter.

116. The method of claim 115, wherein said linear nucleic acid is double stranded.

117. The method of claim 115, wherein said linear nucleic acid is single stranded.

118. A method for identifying a compound which inhibits the proliferation of a prokaryotic cell said method comprising the steps of:
(a) replacing the native promoter of a gene in the chromosome of a prokaryotic cell having an enhanced frequency of homologous recombination with a regulatable fusion promoter comprising at least one promoter that is modified to have altered activity in at least one gram-positive organism said promoter being linked to at least one operator selected from the group consisting of xylO, tetO, trpO, malO, lacO and λc1O, wherein said at least one operator is positioned such that binding of at least one repressor to said at least one operator represses transcription from said fusion promoter; and
(b) comparing the extent of proliferation of a first sample of said cell in the presence of said compound to the extent of proliferation of a second sample of said cell in the presence of said compound, wherein said first sample of said cell has a reduced activity of said fusion promoter activity relative the activity of said fusion promoter in said second sample of said cell and wherein said compound inhibits the proliferation of said cell if the extent of proliferation of said first sample of said cell is substantially less than the extent of proliferation of said second sample of said cell.

119. A method for identifying a compound which inhibits the proliferation of a prokaryotic cell said method comprising the steps of:
(a) replacing the native promoter of a gene in the chromosome of a prokaryotic cell having an enhanced frequency of homologous recombination with a regulatable fusion promoter comprising at least one promoter selected from the group consisting of T5, CP25, P32, P59, P1P2 and PL, said promoter being linked to at least one operator selected from the group consisting of xylO, tetO, trpO, malO, lacO and λc1O, wherein said at least one operator is positioned such that binding of at least one repressor to said at least one operator represses transcription from said fusion promoter; and
(b) comparing the extent of proliferation of a first sample of said cell in the presence of said compound to the extent of proliferation of a second sample of said cell in the presence of said compound, wherein said first sample of said cell has a reduced activity of said fusion promoter activity relative the activity of said fusion promoter in said second sample of said cell and wherein said compound inhibits the proliferation of said cell if the extent of proliferation of said first sample of said cell is substantially less than the extent of proliferation of said second sample of said cell.

120. The method of claim 118, wherein the step of replacing said native promoter comprises introducing a linear nucleic acid comprising a 5′ portion homologous to a first portion of said native promoter, a 3′ portion homologous to a second portion of said native promoter and said fusion promoter disposed between said 5′ portion and said 3′ portion into said cell such that homologous recombination occurs between said 5′ portion and said first portion of said native promoter and between said 3′ portion and said second portion of said native promoter.

121. The method of claim 120, wherein said linear nucleic acid is double stranded.

122. The method of claim 120, wherein said linear nucleic acid is single stranded.

123. A method for identifying a gene which is required for proliferation of a prokaryotic cell said method comprising the steps of:
(a) introducing at least one operator selected from the group consisting of xylO, tetO, trpO, malO, lacO and λc1O into a prokaryotic cell having an enhanced frequency of homologous recombination such that said at least one operator regulates transcription of a target nucleic acid in the chromosome of said cell; and
(b) identifying cells in which the extent of proliferation of said cell when said target nucleic acid is transcribed at a first level is substantially different than the extent of proliferation of said cell when said target nucleic acid is transcribed at a second level, said first level being greater than said second level.

124. The method of claim 123, wherein the step of introducing said at least one operator comprises introducing a linear nucleic acid comprising a 5′ portion homologous to a first portion of the chromosome of said cell, a 3′ portion homologous to a second portion of the chromosome of said cell and said at least one operator disposed between said 5′ portion and said 3′ portion into said cell such that homologous recombination occurs between the 5′ portion and said first portion of the chromosome of said cell and between the 3′ portion and said second portion of the chromosome of said cell.

125. The method of claim 124, wherein said linear nucleic acid is double stranded.

126. The method of claim 124, wherein said linear nucleic acid is single stranded.

127. A method of identifying a compound which inhibits the proliferation of a prokaryotic cell said method comprising the steps of:
(a) obtaining a prokaryotic cell in which transcription of a nucleic acid required for proliferation of said cell is regulated by at least one operator which has been introduced into the chromosome of said cell said at least one operator selected from the group consisting of xylO, tetO, trpO, malO, lacO and λc1O;
(b) sensitizing said cell by growing said cell under conditions in which the level of transcription of said gene is lower than that of a wild type cell;
(c) contacting said sensitized cell with said compound; and
(d) determining the degree to which said compound inhibits the growth of said sensitized cell relative to an unsensitized cell.

128. The method of claim 127, wherein said cell has an enhanced frequency of homologous recombination.

129. The method of claim 128, wherein said linear nucleic acid is single stranded.

130. The method of claim 128, wherein said linear nucleic acid is double stranded.

131. A method of identifying a nucleic acid sequence having promoter activity in Enterococcus faecalis said method comprising the steps of:
(a) inserting a candidate nucleic acid into a vector comprising lacL-lacM reporter genes such that said candidate nucleic acid is upstream of the lacL-lacM reporter genes;
(b) introducing said vector comprising said candidate nucleic acid into Enterococcus faecalis;
(c) detecting expression of the lacL-lacM reporter genes, wherein expression of said lacL-lacM reporter genes indicates that said candidate nucleic acid sequence has promoter activity.

132. The method of claim 131, wherein said detecting step comprises measuring β-galactosidase activity.

133. The method of claim 131, wherein said candidate nucleic acid is a promoter modified to increase activity in a gram-positive organism.

134. The method of claim 131, wherein said lacL-lacM reporter gene is derived from Leuconostoc mesenteroides.

135. The method of claim 131, wherein said vector is pEPEF1.
